(19) United States
(12) Patent Application Publication
(10) Pub. No.: US 2006/0154886 A1
(43) Pub. Date: Jul. 13, 2006

(54) Regulatory elements in the 5' region of the VR1 gene

(75) Inventors: Eberhard Weihe, Marburg (DE); Annette Bieller, Coelbe-Buergeln (DE); Martin Schaefer, Marburg (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/148,303

(22) Filed: Jun. 9, 2005

Related U.S. Application Data
(63) Continuation of application No. PCT/EP03/13522, filed on Dec. 1, 2003.

(30) Foreign Application Priority Data
Dec. 9, 2002 (DE) 102 57 421.9

(51) Int. Cl. A61K 48/00; C07H 21/04; C12N 15/87

(52) U.S. Cl. 514/44; 435/455; 536/23.5

(57) ABSTRACT

A nucleic acid comprising a sequence section which modulates the expression of the VR1 receptor, a vector containing this nucleic acid and a host cell which is transformed with this vector are disclosed, along with related pharmaceutical formulations. Methods for modulating the expression of the VR1 receptor and the use of the nucleic acid or vector for alleviating, preventing or treating pain and for treating sensibility disorders associated with the VR1 receptor are also provided.

### CROSS REFERENCE TO RELATED APPLICATIONS

This application is a continuation of International Patent Application No. PCT/EP2003/013522, filed Dec. 1, 2003, designating the United States of America, and published in German as WO 2004/053120 A2, the entire disclosure of which is incorporated herein by reference. Priority is claimed based on German Patent Application No. 102 57 421.9, filed Dec. 9, 2002.

### FIELD OF THE INVENTION

The present invention relates to a nucleic acid comprising a sequence section which modulates the expression of the VR1 receptor, a vector containing the nucleic acid, a host cell which is transformed with the vector, a method for modulation of the expression of the VR1 receptor and the use of the nucleic acid or vector for prevention, alleviation or treatment of pain and for treatment of sensibility disorders associated with the VR1 receptor.

### BACKGROUND

According to the definition of the IASP (International Association for the Study of Pain), pain is en unpleasant severe sensory and perceptive experience which is associated with actual or possible tissue damage or is described in such categories.

In contrast, nociception relates to the receipt of signals in the CNS which are caused by specialized sensory receptors (nociceptors) and impart information about tissue damage. The isolation and characterization of the vanilloid receptor of subtype 1 (VR1; also called capsaicin receptor), which is expressed in sensory neurones of small diameter, in particular primary sensory neurones of the pain conduction pathway, was a significant advance in the understanding of the molecular basis of nociception in mammals (Caterina et al. (1997) Nature 389: 816 to 824). The cDNA isolated from sensory neurones of rats codes for a polypeptide of 838 amino acids having a predicted molecular weight of 95 kDa and a hydrophobicity profile from which 6 transmembrane domains are predicted. VR1 is activated in vitro by various harmful stimuli, which include plant derivatives, such as the vanilloids capsaicin and resiniferatoxin, as well as certain endogenous agents, e.g. protons, the fatty acid derivative anandamide and inflammatory products of the lipoxygenase metabolic pathway of arachidonic acid. VR1 can moreover also be activated by noxious stimuli (temperatures>42° C.). It has furthermore been found that sensory neurones from VR1^{-/-} mice show a greatly reduced response to these noxious stimuli. The VR1^{-/-} mice respond normally to noxious mechanical stimuli, but show no vanilloid-induced pain behaviour, their detection of noxious heat is impaired, and they show only a low thermal hypersensitivity after an inflammation (Caterina et al. (2000) Science 288: 306 to 313). These observations lead to the conclusion that the VR1 receptor has an important role in the pain event, e.g. for thermal hyperalgesia following tissue damage.

In addition to the cDNA for VR1, the genomic organization of the gene which codes for the vanilloid receptor, in particular the exon/intron structure, has also recently been clarified (Quing Xue et al. (2001) Genomics 76: 14 to 20). The nucleotide sequences of the promoter regions of the VR1 receptor gene of the mouse and of humans are deposited under the GenBank entries AC087118 (in the version of 20th Jul. 2001) and AF168787.

Analgesics described to date e.g. either attack at the level of the modulating systems, in particular the neuronal stimulus conduction, or block specifically the generation of inflammation mediators. Opioids act as specific ligands of the opioid receptors (μ, κ, δ or ORL1). However, these are used only in cases of severe pain (such as in cases of pain in the course of a cancer disease) and have the serious problem of tolerance development, which necessitates an ever higher dosage. For mild to moderate pain, so-called NSAID (“non-steroidal anti-inflammatory drugs”), such as salicylates, are used. These inhibit the cyclooxygenases COX1 and COX2, e.g. Aspirin®, Paracetamol® and Ibuprofen®. However, their pain-alleviating action is usually not sufficient to combat more severe pain.

### SUMMARY OF THE INVENTION

In one embodiment, the present invention is therefore based on the object of providing an alternative system for influencing nociception, in particular for combating pain.

This object is achieved by the embodiments of the present invention as characterized in the claims.

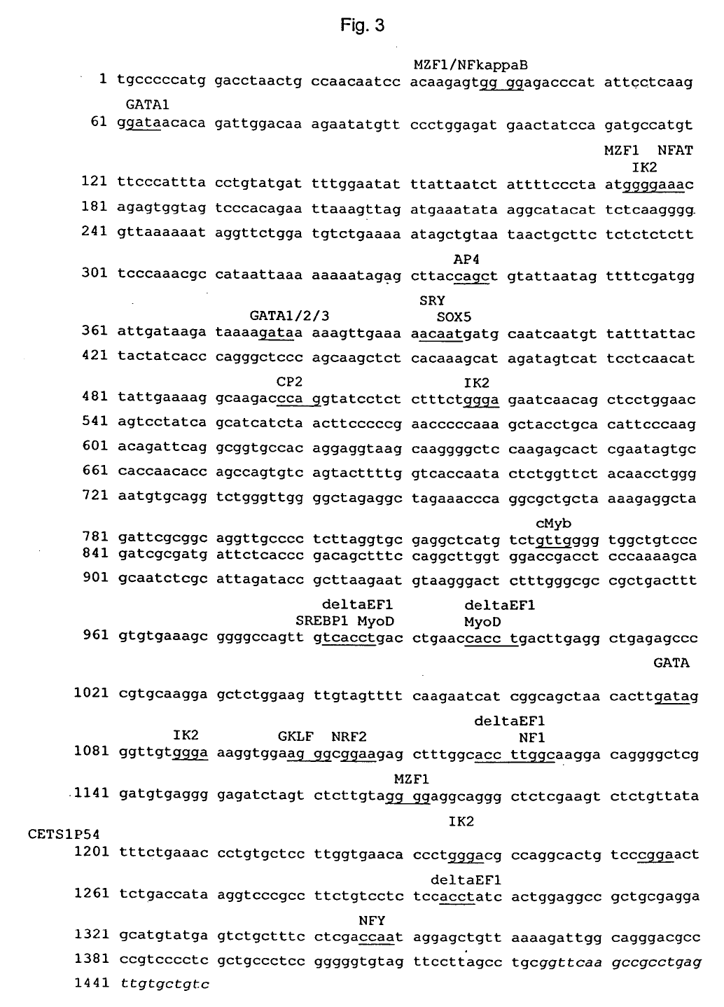

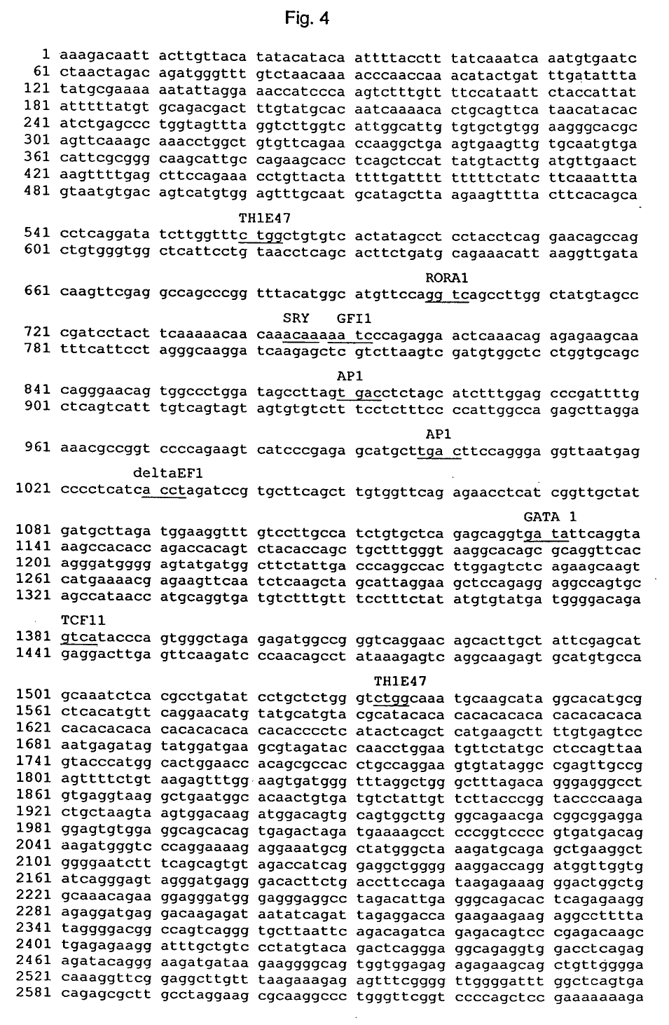

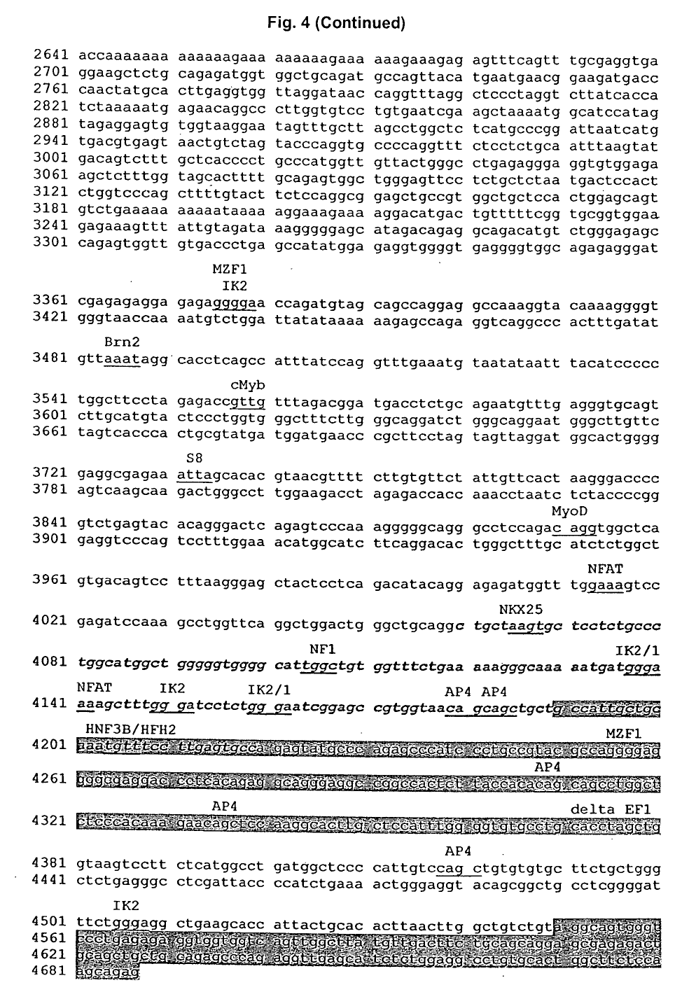

In particular, according to the invention a nucleic acid is provided which contains a sequence section which contains at least one region, which modulates the expression of the VR1 receptor, of the sequence according to FIG. 3 (SEQ ID NO: 7) and/or according to FIG. 4 (SEQ ID NO: 8) and/or according to GenBank Accession Number AL670399, positions 221931 to 223344, and/or according to GenBank Accession Number AL663116, positions 31673 to 36359, and/or according to GenBank Accession Number AF168787, positions 44731 to 43231 (a reverse sequence is deposited under this GenBank Accession Number) and/or according to GenBank Accession Number AF168787, positions 36616 to 33151 (a reverse sequence is deposited under this GenBank Accession Number), or a homologous derivative, allele or fragment thereof which modulates the expression of the VR1 receptor, or a sequence which hybridizes with these sequences under standard conditions.

The expression “region which modulates the expression of the VR1 receptor” means that the corresponding region of the abovementioned nucleotide sequences is capable of intervening in the expression of the vanilloid receptor, in particular during transcription, in a regulating, i.e. either enhancing or inhibiting, manner.

For example, regions having an enhancer function in the above sequences, in particular the regions of the sequences according to FIG. 3 (SEQ ID NO: 7) or FIG. 4 (SEQ ID NO: 8), have an enhancing action, while those which contain repressor binding sites have a reducing action on the expression rate, in particular the transcription rate of the VR1 gene following the 5′ regulatory region shown in FIG. 3 (SEQ ID NO: 7) (in this case starting with exon 1ab), or the transcription rate of the VR1 gene following the 5′ regulatory region shown in FIG. 4 (SEQ ID NO: 8) (in this case starting with either exon 1c or exon 1d), in particular of the gene of the rat. For example, a repressor action of the following factors delta EF1 and GFI1 is known (Funahasi et al. (1993) Development 119(2): 433-446; Zweidler et al. (1996) Mol. Cell. Biol. 08/1996: 4024-4034. Regions having an enhancer function of the sequence according to GenBank Accession Number AL670399, positions 221931 to 223344, or of the sequence according to GenBank Accession Number AL663116, positions 31673 to 36359, likewise have an enhancing action on the expression rate (e.g. transcription rate) of the particular VR1 gene following the 5′ regulatory region contained in these sequences (starting with exon 1ab or exon 1c or exon 1d), while regions having a repressor function have a reducing action on the expression rate of the VR1 gene, in particular the VR1 gene of the mouse. Accordingly, regions having an enhancer function of the sequence according to GenBank Accession Number AF168787, positions 44731 to 43231, or of the sequence according to GenBank Accession Number AF168787, positions 36616 to 33151, have an enhancing action on the expression rate (e.g. transcription rate) of the particular VR1 gene following the 5′ regulatory region contained in these sequences (starting with exon 1ab or exon 1c or exon 1d), while regions having a repressor function have a reducing action on the expression rate of the VR1 gene, in particular the human VR1 gene.

According to a preferred embodiment of the nucleic acid according to the invention, the region which modulates the expression of the VR1 receptor comprises at least one transcription factor binding site present in the sequence of FIG. 3 and/or FIG. 4, in particular a core sequence (binding motif) of such a binding site. Preferred binding sites include the binding motifs for the transcription factors MZF1 (myeloid zinc finger protein 1; cf. e.g. position 39, 173, 1169 according to FIG. 3 (SEQ ID NO: 7)), NFkappaB (nuclear factor-kappaB; cf. e.g. position 39 according to FIG. 3 (SEQ ID NO: 7)), GATA 1/2/3 (GATA-binding factor; cf. e.g. position 62, 376, 1076 according to FIG. 3 (SEQ ID NO: 7)), IK 2 (Ikaros factor 2; cf. e.g. position 174, 517, 1087, 1235 according to FIG. 3 (SEQ ID NO: 7)), NFAT (nuclear factor of activated T-cells; cf. e.g. position 176, 1089 according to FIG. 3 (SEQ ID NO: 7) or position 4013, 4139 according to FIG. 4 (SEQ ID NO: 8)), AP4 (activator protein 4; cf. e.g. position 336 according to FIG. 3 (SEQ ID NO: 7)), SRY (sex-determining region Y gene product; cf. e.g. position 392 according to FIG. 3 (SEQ ID NO: 7)), SOX5 (Sox-5; cf. e.g. position 393 according to FIG. 3 (SEQ ID NO: 7)), CP2 (cf. e.g. position 498 according to FIG. 3 (SEQ ID NO: 7)), cMyb (cf. e.g. position 824 according to FIG. 3 (SEQ ID NO: 7)), SREBP1 (sterol regulatory element-binding protein; cf. e.g. position 982 according to FIG. 3 (SEQ ID NO: 7)), deltaEF1 (delta-crystalline/E2-box factor 1; cf. e.g. position 984, 998, 1118, 1294 according to FIG. 3 (SEQ ID NO: 7)), MyoD (myoblast determining factor; cf. e.g. position 983, 997 according to FIG. 3 (SEQ ID NO: 7)), GKLF (gut-enriched Krüppel-like factor; cf. e.g. position 1099 according to FIG. 3 (SEQ ID NO: 7)), NRF2 (nuclear respiratory factor 2; cf. e.g. position 1104 according to FIG. 3 (SEQ ID NO: 7)), NF1 (nuclear factor 1; cf. e.g. position 1122 according to FIG. 3 (SEQ ID NO: 7)), CETS1P54 (c-Ets (p54); cf. e.g. position 1254 according to FIG. 3 (SEQ ID NO: 7)) and NFY (nuclear factor Y; cf. e.g. position 1346 according to FIG. 3 (SEQ ID NO: 7)).

Transcription factor binding sites which are preferably additionally or alternatively present in the nucleic acid according to the invention are e.g. those for TH1E47 (Thing1/E47 heterodimer; cf. e.g. position 560, 1533 according to FIG. 4 (SEQ ID NO: 8)), RORA1 (RAR-related orphan receptor alpha1; cf. e.g. position 699 according to FIG. 4 (SEQ ID NO: 8)), SRY (cf. e.g. position 744 according to FIG. 4 (SEQ ID NO: 8)), GFI1 (growth factor independence 1; cf. e.g. position 749 according to FIG. 4 (SEQ ID NO: 8)), AP1 (activator protein 1; cf. e.g. position 870, 998 according to FIG. 4 (SEQ ID NO: 8)), deltaEF1 (cf. e.g. position 1030, 4372 according to FIG. 4 (SEQ ID NO: 8)), GATA 1 (cf. e.g. position 1129 according to FIG. 4 (SEQ ID NO: 8)), TCF11 (TCF11/KCR-F1/Nrf1 homodimers; cf. e.g. position 1381 according to FIG. 4 (SEQ ID NO: 8)), MZF1 (cf. e.g. position 3375, 4255 according to FIG. 4 (SEQ ID NO: 8)), IK2/1 (cf. e.g. position 3376, 4137, 4149, 4159, 4505 according to FIG. 4 (SEQ ID NO: 8)), Brn2 (POU factor Brn2; cf. e.g. position 3484 according to FIG. 4 (SEQ ID NO: 8)), cMyb (cf. e.g. position 3557 according to FIG. 4 (SEQ ID NO: 8)), S8 (cf. e.g. position 3731 according to FIG. 4 (SEQ ID NO: 8)), MyoD (cf. e.g. position 3890 according to FIG. 4 (SEQ ID NO: 8)), NKX25 (homeodomain factor Nkx-2.5/Csx; cf. e.g. position 4065 according to FIG. 4 (SEQ ID NO: 8)), NF1 (cf. e.g. position 4104 according to FIG. 4 (SEQ ID NO: 8)), AP4 (cf. e.g. position 4179, 4182, 4308, 4334, 4418 according to FIG. 4 (SEQ ID NO: 8)), HNF3B (hepatocyte nuclear factor-3beta; cf. e.g. position 4204 according to FIG. 4 (SEQ ID NO: 8)) and HFH2 (HNF3 forkhead homologue 2; cf. e.g. position 4204 according to FIG. 4 (SEQ ID NO: 8)). The nucleic acid according to the invention can of course contain one or more such binding sites of one or more transcription factors, by themselves or in any combination.

The nucleic acid defined above is preferred according to the invention as a double-stranded DNA molecule. As such a DNA molecule, in particular if it is present as a relatively short oligodeoxyribonucleotide (ODN), the nucleic acid is a so-called “decoy ODN” or “cis-element decoy”, which contains a sequence which corresponds to or resembles the natural core binding sequence, e.g. one of the abovementioned transcription factors, and to which the particular transcription factor, in particular the abovementioned transcription factors, binds in the cell, in particular in the cell nucleus. The cis-element decoy therefore acts as a molecule for competitive inhibition of the activity of the particular transcription factor.

One aspect of the present invention therefore comprises employing the nucleic acid according to the invention, as an inhibitor of the activity of transcription factors which bind to the 5′ regulatory region of the VR1 gene according to the sequences in FIG. 3 (SEQ ID NO: 7), FIG. 4 (SEQ ID NO: 8), GenBank Accession Number AL670399, positions 221931 to 223344, GenBank Accession Number AL663116, positions 31673 to 36359, GenBank Accession Number AF168787, positions 44731 to 43231, or GenBank Accession Number AF168787, positions 36616 to 33151, as a pharmaceutical formulation. Such proteins, which also include the abovementioned transcription factors, can be inhibited in their action as transcription activators by nucleic acids according to the invention having an action as a cis-element decoy.

The use of double-stranded DNA oligonucleotides (also called cis-decoy or decoy ODN) which contain one or more binding sites for the particular transcription factor(s) is therefore preferred for the specific inhibition of the activity, in particular of the abovementioned transcription factors. Exogenous supply of a large number of transcription factor binding sites, in particular in a number far higher than present in the genome, generates a situation in which a majority of a certain intracellularly present transcription factor binds specifically to the particular cis-element decoy and not to its endogenous target binding sites in the genome. This set-up for inhibition of the binding of transcription factors to their endogenous binding site is also called “squelching”. Squelching of transcription using cis-element decoy has been employed successfully e.g. to inhibit the growth of cells. In this context, DNA fragments which contained specific transcription factor binding sites of the transcription factor E2F were used (Morishita et al. (1995) Proc. Natl. Acad. Sci. USA 92: 5855).

According to the invention, e.g. the sequence of a nucleic acid which binds to the transcription factors C/EBP B, MZF, Nkx 2.5, NF-AT, GATA, MZF, Brn-2, IK2 or AT4 is suitable. C-EBTB binds specifically to the motif with the core sequence GCAA, MZF binds specifically to motifs with the core sequence GGG, Nkx 2.5 binds specifically to motifs with the core sequence TAAT, NF-AT binds specifically to motifs with the core sequence GAAA, GATA binds specifically to sequences with the core motif GATA, Brn-2 binds specifically to core sequences with the motif AAAT, IK2 binds specifically to the motif with the core sequence GGGA and AP4 binds specifically to motifs with the core sequence GAGC. Further specific examples of motifs which can be used according to the invention can be found in the sequences given in Tables 1 to 6 (in each case the last (right-hand) column) in the appendix, the particular core sequence (binding motif) being emphasized in capital letters. The nucleic acid according to the invention as a cis-element decoy can therefore be constructed as an oligomer which contains one or more of the above consensus core binding sequences. The cis-element decoy can of course have a variable size which is significantly greater than the particular core binding sequence and is elongated at the 5′ end and/or at the 3′ end.

Since the nucleic acid as a cis-element decoy is a double-stranded nucleic acid, such a DNA oligonucleotide according to the invention comprises in each case not only the sense or forward sequence but also the complementary antisense or reverse sequence. The particular complementary sequences are not reproduced here, but result from the specific base pairing (A-T, G-C) in DNA molecules in a manner which is easily understandable for a person of skill in the art.

On the basis of the specific base pairing in DNA, the cis-element decoy according to the invention not only can have several binding sites for one or more transcription factors on one strand, but also in each case one or more binding sites can be present in the sense and antisense strand. An expert can therefore see that a large number of sequences can be used as inhibitors e.g. for the abovementioned transcription factors, as long as they meet the conditions described above for consensus core binding sequences and have an affinity for the particular transcription factor.

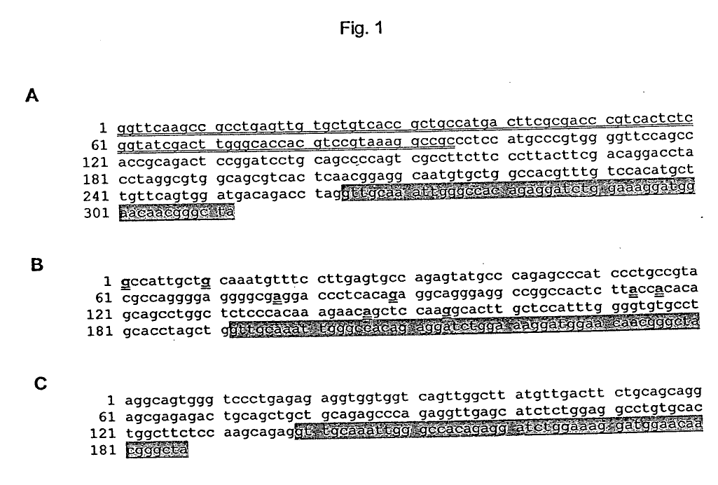

The binding affinity of a double-stranded nucleic acid sequence according to the invention to a transcription factor can be determined by using electrophoretic mobility shift assay (EMSA) (Sambrook et al. (2001) Molecular Cloning: A Laboratory Handbook, Cold Spring Harbour Laboratory Press, Cold Spring Harbour; Krzesc et al. (1999) FEBS Lett. 453: 191). This test is particularly suitable for quality control of the nucleic acid according to the invention when used as a transcription inhibitor of the VR1 gene or for determination of the optimum length of a binding site. EMSA is also suitable for identification of other sequences to which the abovementioned transcription factors or other transcription factors which bind to the sequence shown in FIG. 1. The EMSA test system which is used for isolation of new binding sites is preferably carried out with purified or recombinantly expressed versions of the particular transcription factors, which are employed in EMSA in several alternating rounds of PCR multiplication and selection (Thiesen and Bach (1990) Nucleic Acids Res. 18: 3203).

The transcription of the VR1 gene is modulated by the nucleic acid according to the invention as a cis-element decoy such that this gene is not expressed or is expressed to a reduced extent. According to the present invention, reduced or suppressed expression means that the transcription rate is decreased compared with cells which are not treated with a double-stranded DNA oligonucleotide according to the invention. Such a reduction can be determined e.g. by means of northern blotting (Sambrook et al., supra) or RT-PCR (Sambrook et al., supra).

It is likewise possible according to the invention for the nucleic acid constructed as a cis-element decoy to be employed for increasing the expression rate of the VR1 gene, in that one or more binding sites for a protein which reduces the transcription rate of the VR1 gene (repressor) are present in the cis-element decoy. The factors delta EF1 and GFI1, for which a repressor action is known and which are already mentioned above can be given as examples of such sequences.

The transcription rate of the VR1 gene in cells treated with decoys according to the invention is typically reduced or increased at least 2-fold, in particular 5-fold, particularly preferably at least 10-fold compared with cells which are not treated with a double-stranded DNA oligonucleotide according to the invention.

In a preferred embodiment, the nucleic acid according to the invention used as a cis-element decoy contains one or more, preferably 1, 2, 3, 4 or 5, particularly preferably 1 or 2 binding sites, shown in the sequence of FIG. 1, to which a transcription factor binds specifically. The particular nucleic acid can be prepared by synthesis, or in vitro or intracellularly using molecular biology processes. The particular process is known to an expert (cf. e.g. Sambrook et al., supra).

The length of the nucleic acid according to the invention, in particular of the double-stranded DNA oligonucleotide, is preferably at least as long as a sequence used which binds specifically to a transcription factor which contains one of the core binding sequences contained in the sequences listed above. The nucleic acid according to the invention conventionally comprises about 13 to about 65 bp, preferably about 18 to about 23 bp.

Oligonucleotides are as a rule degraded rapidly in the cell by endo- and exonucleases, in particular DNases and RNases. A decoy nucleic acid according to the invention can therefore be modified in order to stabilize it against enzymatic degradation, so that a high concentration of the double-stranded nucleic acid is ensured in the cell over a relatively long period of time, and the duration of action thereof is thus prolonged. Such a stabilizing can typically be obtained by introduction of one or more modified internucleotide bonds or by introduction of a modified nucleobase.

A nucleic acid modified in this way, in particular a DNA oligonucleotide, does not necessarily contain a modification on each internucleotide bond or each nucleobase. Preferably e.g. the internucleotide bonds at the particular ends of the two oligonucleotides of a cis-element decoy are modified. In this context, the last 6, 5, 4, 3, 2 or the last or another or several internucleotide bond(s) within the last 6 internucleotide bonds can be modified. Furthermore, various modifications of the internucleotide bonds can be introduced into the nucleic acid, and the double-stranded DNA oligonucleotides formed therefrom can be tested for sequence-specific binding to the desired transcription factor(s) using the standard EMSA test system. The EMSA test system allows determination of the binding constant of the nucleic acid according to the invention and thus determination of whether the affinity has been changed by the modification. The cis-element decoys which still show adequate binding can be selected, adequate binding meaning at least about 50% or at least about 75%, particularly preferably about 100% of the binding of the non-modified nucleic acid.

Nucleic acids according to the invention, in particular cis-element decoys, with (a) modified internucleotide bond(s) or modified nucelobases which still show adequate binding can be investigated as to whether they are more stable in the cell than the non-modified molecules. For this, the cells transfected with the nucleic acid according to the invention are investigated at various points in time for the amount of nucleic acid still present at that time. The methods known to an expert can be used for this, e.g. Southern blotting techniques (Sambrook et al., supra) or DNA chip array techniques (U.S. Pat. No. 5,837,466). A successfully modified nucleic acid according to the invention, e.g. a cis-element decoy according to the invention, has a half-life in the cell which is longer than that of the non-modified molecule, preferably at least a half-life of about 48 hours, more preferably of at least about 4 days, particularly preferably of at least about 7 days.

Suitable modified internucleotide bonds are summarized e.g. in Uhlmann and Peiman ((1990) Chem. Rev. 90: 544). Modified internucleotide phosphate moieties and/or non-phosphorus bridges which can be employed according to the invention contain e.g. methyl phosphonate, phosphorothioate, phosphorodithioate, phosphoramidate or phosphate ester, while non-phosphorus internucleotide analogues contain e.g. siloxane bridges, carbonate bridges, carboxymethyl ether bridges, acetamidate bridges and/or thioether bridges. Modified nucleobases which may be mentioned are e.g. 7-deazaguanosine, 5-methylcytosine and inosine.

A further possibility for stabilizing the nucleic acid according to the invention is the introduction of structural features which increase the half-life of the nucleic acid into the nucleic acid according to the invention. Such structures, which contain e.g. hairpin and dumbbell DNA, are disclosed in U.S. Pat. No. 5,683,985. At the same time, modified internucleotide phosphate moieties and/or non-phosphorus bridges and/or modified nucleobases can be introduced into the nucleic acid according to the invention together with the structures mentioned. The resulting nucleic acids can be investigated for binding and stability in the test system described above.

According to a further embodiment of the nucleic acid according to the invention, the regulatory sequence section defined above comprises the sequence shown in FIG. 3 (SEQ ID NO: 7), in particular the nucleotides of the sequence shown in positions 1 to 1423 of this figure (i.e. up to the start of the gene section coding the cDNA, which starts with exon 1a), or a derivative, allele or fragment thereof which modulates the expression of the VR1 receptor, or a sequence which hybridizes with this under standard conditions.

According to a further embodiment of the nucleic acid according to the invention, the regulatory sequence section defined above comprises the sequence shown in FIG. 4 (SEQ ID NO: 8), in particular the nucleotides of the sequence shown in positions 1 to 4549 in this figure (i.e. up to the start of the gene section coding the cDNA, which starts with exon 1d), or a derivative, allele or fragment thereof which modulates the expression of the VR1 receptor, or a sequence which hybridizes with this under standard conditions.

According to a further embodiment of the nucleic acid according to the invention, the regulatory sequence section defined above comprises the sequence shown in FIG. 4 (SEQ ID NO: 8), in particular the nucleotides of the sequence shown in positions 1 to 4190 in this figure (i.e. up to the start of the gene section coding the cDNA, which starts with exon 1c), or a derivative, allele or fragment thereof which modulates the expression of the VR1 receptor, or a sequence which hybridizes with this under standard conditions.

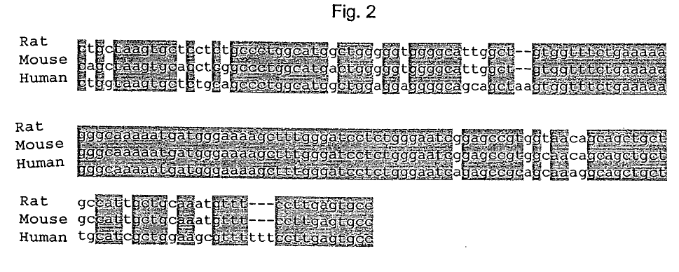

According to a further preferred embodiment, the regulatory sequence section of the nucleic acid according to the invention comprises the nucleotides of the sequence shown in positions 4060 to 4219 in FIG. 4 (SEQ ID NO: 8), or a derivative, allele or fragment thereof which modulates the expression of the VR1 receptor, or a sequence which hybridizes with this under standard conditions. The above sequence section comprising the nucleotides of the sequence shown in positions 4060 to 4219 in FIG. 4 (SEQ ID NO: 8) is distinguished by a high conservation between various species, e.g. rat, mouse and humans (cf. also FIG. 2) and therefore plays a prominent role in regulation of the expression of the VR1 receptor.

The present invention also provides a nucleic acid which codes for VR1, in particular an (m)RNA or (c)DNA, comprising one of the sequences shown in FIG. 1A (SEQ ID NO: 1), B (SEQ ID NO: 2) and C (SEQ ID NO: 3) (wherein in the case of an RNA for each t (thymidine) present in FIGS. 1A, B and C there is a u (uracil)), or a derivative, allele or fragment thereof which codes for VR1, or a sequence which hybridizes with this under standard conditions. Preferred embodiments of this further nucleic acid of the present invention contain nucleotides 1 to 263 of FIG. 1A (exon 1ab), 1 to 191 of FIG. 1B (exon 1c) or 1 to 138 of FIG. 1C (exon 1d) or a derivative, allele or fragment thereof which codes for VR1, or a sequence which hybridizes with this under standard conditions.

Functionally homologous derivatives, alleles or fragments according to the invention of the nucleic acid according to the invention and also unfunctional derivatives, alleles, analogues or fragments can be prepared by standard methods (Sambrook et al., supra). In these methods, one or more nucleotides are inserted, deleted or substituted in the corresponding sequences.

Fragments of the nucleic acid according to the invention are, in particular, those sequence sections which have a sequence which contains one or more of the transcription factor binding sites shown in FIG. 3 (SEQ ID NO: 7), FIG. 4 (SEQ ID NO: 8), the sequence according to GenBank Accession Number AL670399 (positions 221931 to 223344), the sequence according to GenBank Accession Number AL663116 (positions 31673 to 36359), the sequence according to GenBank Accession Number AF168787 (positions 44731 to 43231) or the sequence according to GenBank Accession Number AF168787 (positions 36616 to 33151). Transcription factor binding sites which are preferred here are shown in Tables 1 to 6 in the appendix.

Derivatives of nucleic acids according to the invention or fragments thereof are e.g. molecules described above with internucleotide bond or nucleobase modifications.

Functionally homologous allele variants in the context of the present invention are variants which have at least 60%, preferably at least 70%, more preferably at least 90% homology. Allele variants include, in particular, those functional or unfunctional variants which are obtainable by deletion, insertion or substitution of nucleotides from the sequence according to FIG. 3 (SEQ ID NO: 7), the sequence according to FIG. 4 (SEQ ID NO: 8), the sequence according to GenBank Accession Number AL670399 (positions 221931 to 223344), the sequence according to GenBank Accession Number AL663116 (positions 31673 to 36359), the sequence according to GenBank Accession Number AF168787 (positions 44731 to 43231) or the sequence according to GenBank Accession Number AF168787 (positions 36616 to 33151), where, however, the regulatory function in respect of the expression of the VR1 receptor is substantially retained.

Homologous nucleotide sequences or those of related sequence can be isolated from mammalian species, including humans, by the usual processes by homology screening, by hybridization with a probe of the nucleic acid sequence according to the invention or parts thereof. Functional equivalents are also to be understood as meaning homologues of the sequence according to FIG. 3 (SEQ ID NO: 7), the sequence according to FIG. 4 (SEQ ID NO: 8), the sequence according to GenBank Accession Number AL670399 (positions 221931 to 223344), the sequence according to GenBank Accession Number AL663116 (positions 31673 to 36359), the sequence according to GenBank Accession Number AF168787 (positions 44731 to 43231) or the sequence according to GenBank Accession Number AF168787 (positions 36616 to 33151), e.g. their homologues from other mammals, shortened sequences, single-stranded DNA or RNA. Such functional equivalents can be isolated from other vertebrates, in particular mammals, starting from the nucleotide sequence according to FIG. 3 (SEQ ID NO: 7), the nucleotide sequence according to FIG. 4 (SEQ ID NO: 8) (SEQ ID NO: 7), the nucleotide sequence according to GenBank Accession Number AL670399 (positions 221931 to 223344), the nucleotide sequence according to GenBank Accession Number AL663116 (positions 31673 to 36359), the nucleotide sequence according to GenBank Accession Number AF168787 (positions 44731 to 43231) or the nucleotide sequence according to GenBank Accession Number AF168787 (positions 36616 to 33151), or parts of these sequences, e.g. with conventional hybridization methods or by the PCR technique. All the sequences which hybridize with the abovementioned sequences are therefore also disclosed according to the invention. These sequences hybridize with the nucleic acid sequences according to the invention under standard conditions. Short oligonucleotides of the conserved regions are advantageously used for the hybridization. However, longer fragments of the nucleic acids according to the invention or the complete sequence can also be used for the hybridization.

These standard conditions vary according to the nucleic acid sequence used (oligonucleotide, longer fragment or complete sequence) and according to what type of nucleic acid (DNA or RNA) is used for the hybridization. Thus e.g. the melting temperatures for DNA:DNA hybrids are approximately 10° C. lower than those of DNA:RNA hybrids of the same length. Standard conditions are to be understood as meaning e.g., depending on the nucleic acid, temperatures of between 42° C. and 58° C. in an aqueous buffer solution having a concentration of between 0.1 to 5×SSC (1×SSC=0.15 M NaCl, 15 mM sodium citrate, pH 7.2) or additionally in the present of 50% formamide, such as e.g. 42° C. in 5×SSC, 50% formamide. The hybridization conditions for DNA:DNA hybrids are advantageously 0.1×SSC and temperatures of between about 20° C. to 45° C., preferably between about 30° C. to about 45° C. For DNA: RNA hybrids the hybridization conditions are advantageously 0.1×SSC and temperatures of between about 30° C. to 55° C., preferably between about 45° C. to about 55° C. These temperatures stated for the hybridization are examples of calculated melting temperature values for a nucleic acid having a length of approx. 100 nucleotides and a G+C content of 50% in the absence of formamide. The experimental conditions for the DNA hybridization are known to an expert from relevant textbooks of genetics, e.g. Sambrook et al., supra, and can be calculated according to known formulae, e.g. depending on the length of the nucleic acids, the nature of the hybrids or the G+C content. An expert can find further information on the hybridization in the following textbooks: Ausubel et al. (ed.), 1989, Current Protocols in Molecular Biology, John Wiley & Sons, New York; Hames and Higgins (ed.), 1985, Nucleic Acids Hybridization: A Practical Approach, IRL Press at Oxford University Press, Oxford; Brown (ed.), 1991, Essential Molecular Biology: A Practical Approach, IRL Press at Oxford University Press, Oxford.

According to the invention, derivatives are furthermore also to be understood as meaning variants which have preferably been modified at the 3′ end. Such markings or “tags” which are known in the literature are e.g. hexa-histidine anchors or epitopes which can be recognized as antigens of various antibodies (Studir et al. (1990) Meth. Enzymol, 185: 60 to 89, and Ausubel et al., supra).

All the methods familiar to an expert for the preparation, modification and/or detection of nucleic acid sequences according to the invention, which can be carried out in vivo, in situ or in vitro, are moreover possible (PCR (cf. Innis et al,. PCR Protocols: A Guide to Methods and Applications) or chemical synthesis). By appropriate PCR primers e.g. new functions can be introduced into a nucleotide sequence according to the invention, e.g. restriction sites. By this means, sequences according to the invention can be appropriately designed for transfer into cloning vectors.

The present invention also provides a vector or a recombinant nucleic acid construct which contains a nucleic acid (sequence) defined above, typically a DNA sequence. In this context, the nucleic acid (sequence) according to the invention can be linked functionally with at least one further genetic regulation element, e.g. transcription signals. Host organisms or host cells, e.g. cell cultures from mammalian cells, can then be transformed with vectors produced in such a manner. A vector according to the invention containing the nucleotide sequence defined above can contain e.g. the cDNA sequence, preferably downstream of the nucleotide sequence according to the invention, which codes for the VR1 receptor. The section which codes for the VR1 receptor can of course also code for a functionally homologous derivative, allele or fragment of the VR1 receptor, or a sequence which hybridizes with this under standard conditions.

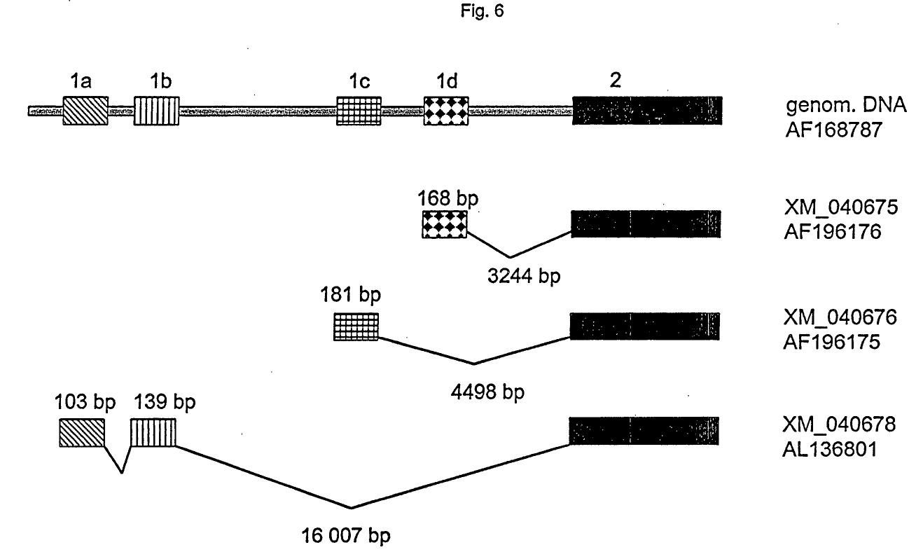

Preferred DNA sequences which code for a functionally homologous protein of the VR1 receptor are identical in sequence to at least 60%, preferably at least 80% and still more preferably at least 95% with the cDNA sequence which results from the corresponding data in the GenBank entry AF327067 (genomic sequence of the VR1 gene of the rat). The functionally homologous partial sequences resulting from these DNA sequences can also be expressed with the aid of the vector according to the invention. Moreover, all native splicing variants of the VR1 cDNA sequence also belong to the scope of the present invention. Embodiments of the VR1 cDNA which are preferred according to the invention start e.g. with exon 1ab, exon 1a or exon 1d (cf. also FIG. 6).

The vector according to the invention or the nucleic acid construct according to the invention can also code for an allele variant or iso-form of the VR1 receptor. In the context of the present invention, allele variants are understood as variants which have 60-100% homology at the amino acid level, preferably 70-100%, very particularly preferably 90-100%. Allele variants include in particular those functional or unfunctional variants which are obtainable by deletion, insertion or substitution of nucleotides from the cDNA sequence which codes for the VR1 receptor (e.g. starting with exon 1ab, exon 1c or exon 1d), the essential biological property as a ligand-controlled cation channel being retained.

A vector according to the invention or a nucleic acid construct according to the invention containing the nucleic acid sequence according to the invention or derivatives, variants, homologues or fragments thereof, also a protein with the function of the VR1 receptor and also an unfunctional variant, e.g. a double negative mutant (DN mutant) can moreover be used in a therapeutically or diagnostically suitable form. Vector systems or oligonucleotides which elongate the sequences which code for the VR1 construct by certain nucleotide sequences and therefore code for modified polypeptides which serve e.g. for easier purification can be used to generate such recombinant VR1 proteins.

A vector according to the invention can furthermore comprise further regulation elements linked functionally with the abovementioned elements, e.g. translation start or translation stop signals. Depending on the desired use, this linking leads to a native expression rate or also to an increase in or lowering of the native gene expression.

The vector according to the invention, e.g. an expression vector for expression of functional or unfunctional VR1 receptors, can comprise further regulation sequences, which are contained e.g. in promoters such as the cos, tac, trp, tet, trp-tet, lpp, lac, lpp-lac, laclq, T7, T5, T3, gal, trc, ara, SP6, I-PR- or even I-PL promoter. Further advantageous regulation sequences are contained e.g. in the Gram-positive promoters, such as amy and SPO2, in the yeast promoters, such as ADC1, MFa, AC, P-60, CYC1 and GAPDH, or in mammalian promoters, such as CaM kinase II, CMV, nestin, L7, BDNF, NF, MBP, NSE, β-globin, GFAP, GAP43, tyrosine hydroxylase, kainate receptor subunit 1 and glutamate receptor subunit B. All the natural promoters with their regulation sequences can in principle be used with the regulatory nucleic acid sequence according to the invention, e.g. the abovementioned regulation sequences, for a(n) (expression) vector according to the invention.

Synthetic promoters can moreover also advantageously be combined. These regulatory sequences are to render possible controlled expression e.g. of VR1 receptor constructs. This can mean e.g., depending on the host organism, that the gene is expressed or overexpressed only after induction, or that it is expressed and/or overexpressed immediately. In this context, the regulatory sequences or factors can preferably positively influence and thereby increase the expression. Thus, an enhancement of the regulatory elements can advantageously take place at the transcription level in that potent transcription signals, such as promoters and/or “enhancers” are used. In addition, however, an enhanced translation is also possible, in that e.g. the stability of the mRNA is improved.

All the elements familiar to the expert which can influence the expression at the transcription and/or translation level are called regulation sequences. In particular, in this context in addition to promoter sequences so-called “enhancer” sequences, which can have the effect of an increased expression via an improved interaction between RNA polymerase and DNA, are to be emphasized. The so-called “locus control regions”, “silencers” or particular partial sequences thereof may be mentioned by way of example as further regulation sequences. These sequences can advantageously be used for tissue-specific expression. So-called “terminator sequences” are also advantageously present in a(n) (expression) vector according to the invention, and according to the invention are subsumed under the term “regulation sequence”.

The term “vector” includes both recombinant nucleic acid constructs or gene constructs, as described above, and complete vector constructs, which typically also contain further elements, in addition to nucleotide sequences according to the invention and any further regulation sequences. These vector constructs or vectors can be used e.g. for expression of the VR1 receptor in a suitable host organism. Advantageously, at least one nucleic acid according to the invention containing an abovementioned sequence section is inserted into a host-specific vector. Suitable vectors are well-known to an expert and can be found e.g. from “Cloning Vectors” (ed. Pouwls et al., Elsevier, Amsterdam-New York-Oxford, 1985, ISBN 0 444 904018). Apart from plasmids, vectors are also to be understood as meaning all other vectors known to an expert, such as e.g. phages, viruses, such as SV40, CMV, baculovirus, adenovirus and Sindbis virus, transposons, IS elements, phasmids, phagemids, cosmids and linear or circular DNA. These vectors can be replicated autonomously in the host organisms or replicated chromosomally. Linear DNA is typically used for the integration into the genome of mammals.

The expression with the VR1 receptor DNA sequences according to the invention coupled to regulatory nucleic acid sequences can advantageously be increased by increasing the number of gene copies and/or by enhancing regulatory factors which have a further positive influence on gene expression. Thus, an enhancement of regulatory elements can preferably take place at the transcription level in that further transcription signals, such as promoters and enhancers, are used. In addition, however, an enhancement of the translation is also possible, e.g. by improving the stability of the mRNA or increasing the reading efficiency of this mRNA at the ribosomes. If the number of copies is increased, the nucleic acid sequences in the case of homologous genes can be incorporated e.g. into a nucleic acid fragment or into a vector, which preferably contains a regulatory gene sequence assigned to the particular genes or a promoter activity of analogous action. In particular, such further regulatory sequences which enhance the gene expression are used.

Nucleic acid sequences according to the invention can be cloned into an individual vector together with the sequences which code for interacting or for potentially interacting proteins, and then expressed in vitro in a host cell or in vivo in a host organism. Alternatively, any of the potentially interacting nucleic acid sequences and the sequence which codes for a VR1 gene construct can also be introduced into in each case an individual vector, and these can be introduced separately into the particular organism via conventional methods, e.g. transformation, transfection, transduction, electroporation or particle gun.

In a further advantageous embodiment, at least one marker gene (e.g. antibiotics resistance genes and/or genes which code for a fluorescent protein, in particular GFP) can be incorporated into a(n) (expression) vector according to the invention, in particular a complete vector construct.

The present invention also provides host cells, (eventually excluding or including human germ cells and human embryonic stem cells), which are transformed with a nucleic acid according to the invention and/or a vector according to the invention. Possible host cells are all cells of a pro- or eukaryotic nature, e.g. from bacteria, fungi or yeasts or plant or animal cells. Preferred host cells are bacterial cells, such as Escherichia coli, Streptomyces, Bacillus or Pseudomonas, eukaryotic microorganisms, such as Aspergillus or Saccharomyces cerevisiae or ordinary baker's yeast (Stinchcomb et al. (1997) Nature 282: 39).

In a preferred embodiment, however, cells from multicellular organisms are chosen for transformation by means of nucleic acids and/or vectors according to the invention. This is effected e.g. in the case of expression of VR1 constructs, due to a possibly desired glycosylation (N- and/or O-coupled) of the coded VR1 construct. This function can be implemented in a suitable manner in higher eukaryotic cells—compared with prokaryotic cells. In principle, any higher eukaryotic cell culture is available as the host cell, although cells from mammals, e.g. apes, rats, hamsters, mice or humans, are very particularly preferred. A large number of established cell lines are known to the expert. The following cell lines are mentioned in a list which is in no way conclusive: 293T (embryonic kidney cell line) (Graham et al., J. Gen. Virol. 36: 59 (1997), BHK (baby hamster kidney cells), CHO (cells from the hamster ovaries, Urlaub and Chasin, Proc. Natl. Accad. Sci. USA 77: 4216, (1980)), HeLa (human carcinoma cells) and further cell lines—in particular established for laboratory use—e.g. HEK293, SF9 or COS cells. Human cells, in particular neuronal stem cells and cells of the “pain pathway”, preferably primary sensory neurones, are very particularly preferred. Human cells, in particular autologous cells of a patient, after (above all ex vivo) transformation with nucleic acids according to the invention or vectors according to the invention, are very particularly suitable as pharmaceutical formulations for e.g. gene therapy purposes, that is to say after carrying out a cell removal, optionally ex vivo expansion, transformation, selection and final retransplantation into the patient.

The combination of a host cell and a vector according to the invention which matches the host cells, such as plasmids, viruses or phages, such as e.g. plasmids with the RNA polymerase/promoter system, the phages λ, Mu or other temperate phages or transposons and/or further advantageous regulatory sequences, forms a host cell according to the invention which can serve as an expression cell system in combination with the regulatory nucleic acid sequence according to the invention. Preferred expression systems according to the invention based on host cells according to the invention are e.g. the combination of mammalian cells, e.g. CHO cells or neuronal cells, and vectors, such as e.g. pcDNA 3neo vector or e.g. HEK293 cells and CMV vectors, which are particularly suitable for mammalian cells

The subjects according to the invention are thus suitable as pharmaceutical formulations on the one hand for inhibition of nociception, e.g. on the basis of the reduction in the transcription of the VR1 receptor by means of cis-element decoy molecules according to the invention or by enhanced expression of an unfunctional variant of the VR1 receptor with the aid of a vector, comprising the total regulatory nucleic acid sequence shown in FIG. 3 (SEQ ID NO: 7) or FIG. 4 (SEQ ID NO: 8) or the total regulatory nucleic acid sequence according to GenBank Accession Number AL670399 (positions 221931 to 223344), according to GenBank Accession Number AL663116 (positions 31673 to 36359), according to GenBank Accession Number AF168787 (positions 44731 to 43231) or according to GenBank Accession Number AF168787 (positions 36616 to 33151), or suitable sections, alleles or derivatives of these sequences. On the other hand, the subjects according to the invention can be used to treat a sensibility disorder associated with the VR1 receptor which leads to reduced sensibility of the particular organism, in particular to a hyp- or analgesia, by means of the subjects according to the invention, e.g. by introducing a nucleic acid according to the invention into the cells of the particular organism in combination with the cDNA which codes for the VR1 receptor, in order thus, e.g. in the case of abnormally reduced or absent expression of the endogenous VR1 receptor, to ensure expression of a functional VR1 receptor construct.

The present invention consequently includes the use of the abovementioned subjects for treatment or for the preparation of a pharmaceutical formulation for treatment, alleviation and/or prevention of pain, in particular acute or chronic pain, and also the use for treatment or for the preparation of a pharmaceutical formulation for treatment of sensibility disorders associated with the VR1 receptor, in particular for treatment of hyperalgesia, hypalgesia or analgesia, neuralgia or myalgia.

Pharmaceutical formulations according to the invention and pharmaceutical formulations prepared using the subjects according to the invention optionally comprise, in addition to the subjects defined above, one or more suitable auxiliary substances and/or additives. Pharmaceutical formulations according to the invention can be administered as a liquid pharmaceutical formulation form in the form of an injection solution, drops or juices or as semi-solid pharmaceutical formulation forms in the form of granules, tablets, pellets, patches, capsules, plasters or aerosols, and optionally comprise, in addition to at least one of the subjects according to the invention, carrier materials, fillers, solvents, diluents, dyestuffs and/or binders, depending on the pharmaceutical form. The choice of auxiliary substances and the amounts thereof to be employed depend on whether the pharmaceutical formulation is to be administered orally, perorally, parenterally, intravenously, intraperitoneally, intradermally, intramuscularly, intranasally, buccally, rectally or topically, to the mucous membranes, the eyes etc. Formulations in the form of tablets, coated tablets, capsules, granules, drops, juices and syrups are suitable for oral administration, and solutions, suspensions, easily reconstitutable dry formulations and sprays are suitable for parenteral, topical and inhalatory administration. Subjects according to the invention in a depot in dissolved form or in a plaster, optionally with the addition of agents which promote penetration through the skin, are suitable formulations for percutaneous administration. Formulation forms which can be used orally or percutaneously can release the subjects according to the invention in a delayed manner. The amount of active compound to be administered to a patient varies as a function of the weight of the patient, the mode of administration, the indication and the severity of the disease. 2 to 500 mg/kg of body weight of at least one subject according to the invention are conventionally administered. If the pharmaceutical formulation is to be used in particular for gene therapy, a physiological saline solution, stabilizers, protease or DNase inhibitors etc. are recommended e.g. as suitable auxiliary substances or additives.

Examples of suitable additives and/or auxiliary substances, e.g. in the use of the nucleic acid according to the invention as a cis-element decoy, which are to be mentioned are lipids, cationic lipids, polymers, liposomes, nucleic acid aptamers, peptides and proteins which are bound to DNA (or synthetic peptide-DNA molecules) in order e.g. to increase the introduction of nucleic acids into the cell, in order to direct the pharmaceutical formulation mixture to only a subgroup of cells, in order to prevent the degradation of the nucleic acid according to the invention in the cell, in order to facilitate storage of the pharmaceutical formulation mixture before use etc. Examples of peptides and proteins or synthetic peptide-DNA molecules are e.g. antibodies, antibody fragments, ligands and adhesion molecules, all of which can be modified or non-modified. Auxiliary substances which e.g. stabilize the cis-element decoys in the cell are e.g. nucleic acid-condensing substances, such as cationic polymers, poly-L-lysine or polyethyleneimine.

In the case of local use of subjects according to the invention, e.g. cis-element decoys, administration is by injection, catheter, suppository, aerosols (nasal or oral spray, inhalation), trocars, projectiles, pluronic gels, polymers providing sustained release of pharmaceutical formulations, or any other device which renders local access possible. Ex vivo use of the pharmaceutical formulation mixture according to the invention used for treatment of the abovementioned indications also allows local access.

Subjects according to the invention can optionally be combined with e.g. at least one further painkiller in a composition as a pharmaceutical formulation (active compound) mixture. Subjects according to the invention can be combined in this manner e.g. in combination with opiates and/or synthetic opioids (e.g. morphine, levomethadone, codeine, tramadol, bupremorphine (buprenorphine)) and/or NSAID (e.g. diclofenac, ibuprofen, paracetamol), e.g. in one of the administration forms disclosed above or also in the course of a combined therapy in separated administrations in each case with optionally a different formulation in a therapy plan appropriately designed medically to suit the requirements of the particular patient. The use of such compositions as pharmaceutical formulation mixtures with e.g. established analgesic for treatment (or for the preparation of pharmaceutical formulations for treatment) of the medical indications disclosed here is preferred.

The present invention also includes a method for modulation of the expression of the VR1 receptor or optionally other receptor genes or genes, comprising introduction of the nucleic acid according to the invention or of the vector into a cell containing the VR1 gene.

The present invention furthermore also includes a method for treatment of the abovementioned indications, comprising administration of at least one subject according to the invention or of a pharmaceutical formulation described above to a patient who requires such an active compound. The preferred administration routes, amounts of the active compounds or of the pharmaceutical formulation etc. which can be used in the treatment method according to the invention have already been described above. “Patients” in the context of the present invention are, in addition to humans, also animals, in particular rodents, e.g. mouse, rat, guinea pig and rabbit, and domestic or stock animals, e.g. chicken, goose, duck, goat, sheep, pig, cattle, horse, dog and cat.

In the use according to the invention or in the treatment method using the subjects according to the invention, the nucleic acid derived from the sequence shown in FIG. 3 (SEQ ID NO: 7) or FIG. 4 (SEQ ID NO: 8) or the corresponding vector or the corresponding host cell is suitable in particular for use in rats. In this context, the subjects derived from the sequence shown in FIG. 3 (SEQ ID NO: 7) are particularly suitable for regulation of the expression of the VR1 gene and disorders associated therewith in the kidney, brain and/or spinal ganglia (or corresponding culture cells or cell lines), while the subjects derived from the sequence shown in FIG. 4 (SEQ ID NO: 8) are particularly suitable for influencing VR1 gene expression in spinal ganglia (or corresponding culture cells or cell lines).

In the use according to the invention or in the treatment method using the subjects according to the invention, the nucleic acid derived from the sequence according to GenBank Accession Number AL670399, positions 221931 to 223344, or from the sequence according to GenBank Accession Number AL663116, positions 31673 to 36359, or the corresponding vector or the corresponding host cell is suitable in particular for use in mice. In this context, the subjects derived from the sequence according to GenBank Accession Number AL670399, positions 221931 to 223344 are particularly suitable for regulation of the expression of the VR1 gene and disorders associated therewith in the kidney, brain and/or spinal ganglia (or corresponding culture cells or cell lines), while the subjects derived from the sequence according to GenBank Accession Number AL663116, positions 31673 to 36359, are particularly suitable for influencing VR1 gene expression in spinal ganglia (or corresponding culture cells or cell lines).

In the use according to the invention or in the treatment method using the subjects according to the invention, the nucleic acid derived from the sequence according to GenBank Accession Number AF168787, positions 44731 to 43231, or the sequence according to GenBank Accession Number AF168787, positions 36616 to 33151, or the corresponding vector or the corresponding host cell is suitable in particular for use in humans. In this context, the subjects derived from the sequence according to GenBank Accession Number AF168787, positions 44731 to 43231 are particularly suitable for regulation of the expression of the VR1 gene and disorders associated therewith in the kidney, brain and/or spinal ganglia (or corresponding culture cells or cell lines), while the subjects derived from the sequence according to GenBank Accession Number AF168787, positions 36616 to 33151, are particularly suitable for influencing VR1 gene expression in spinal ganglia (or corresponding culture cells or cell lines).

The present invention also provides detection methods for a transcription factor, preferably having a high throughput. For this, the regulatory proteins, e.g. transcription factors, which bind to the 5′ regulatory region according to the invention are detected by mutual interactions. This can be effected e.g. by the methods of western blotting, gel shift tests or tests with reporter genes. Such a method can preferably also be carried out on the basis of an ELISA, also in the context of a high throughput method. The conventional ELISA is modified in this context as follows. The e.g. transcription factor to be captured is not captured by an antibody, but rather by a double-stranded oligonucleotide probe which corresponds to the 5′ regulatory region, according to the invention, of the VR1 gene or a section thereof of at least 5, preferably at least 10 nucleotides in length. The double-stranded probes are preferably bound to a substrate, e.g. a microtitre plate. Captured proteins (where these are known as regulator proteins for the 5′ region of VR1) which bind the nucleotide probe can be detected e.g. by corresponding antibodies, e.g. radioactively or fluorescently labelled or those conjugated by horseradish peroxidase (subsequent dyestuff reaction), directed against the captured proteins. In this manner, overexpression and underexpression of the transcription factors in a probe, e.g. in cell extracts, can be detected and corresponding diagnostic findings can be obtained, optionally preventively.

The figures show:

FIG. 1 shows sequences of 5′ RACE fragments which, starting from mRNA from spinal ganglia of the rat, were obtained with gene-specific primers which hybridize in exon 2 of the VR1 cDNA. 3 types of RACE fragments are shown, which contain 49 nucleotides of exon 2 in the 3′ region (AF029310, shaded in grey), but differ in their 5′ sequences. The sequence of the primer rVR72 is underlined. The sequence of the RACE fragment 1ab (A) contains two exons (1a and 1b) in the 5′ region. Exon 1a is double-underlined. Exon 1 shown for the RACE fragment 1c (B) was isolated in various lengths. The start points of the various RACE fragments 1c are shown in bold and double-underlined. The RACE fragment in FIG. 1C contains exon 1d 138 bp in size. The sequences of exons 1a, 1b, 1c und 1d are contained in the genomic sequence of the rat with Accession Number AC126839 [position 53696-53790 (exon 1a), 71745-71912 (exon 1b), position 87717-87907 (exon 1c) and position 88077-88214 (exon 1d)].

FIG. 2 shows a comparison of sequences of the highly conserved DNA region in the 5′ region of exon 1c of the VR1 gene. The sequence sections of the rat [AC126839, position 87587-87746], mouse [AL663116, position 35875-36034] and humans [AF 168787, position 32580-32416] are shown. Identical nucleotides are shaded in grey.

FIG. 3 (SEQ ID NO: 7) shows the genomic sequence in the 5′ region upstream of exon 1a of the VR1 gene of the rat. The sequence was isolated using the GenomeWalker Kit (Clontech) and is contained in the genomic sequence of the rat with the databank number AC126839 [position 52273-53722]. The first nucleotides of the RACE fragment 1ab are shown in italics. DNA binding sites for transcription factors, which are located at the same sequence position both in the rat and in the mouse, are underlined.

FIG. 4 (SEQ ID NO: 8) shows the genomic VR1 sequence in the rat in the 5′ direction of exon 1d. The sequence shown is contained in the genome sequence of the rat with Accession Number AC126839 (position 83528-88214). Exons 1c and 1d are shaded in grey. Die GenomeWalker fragments are located at position 1 to 4361. DNA binding sites for transcription factors, which are located at the same sequence position both in the rat and in the mouse, are underlined. The sequence section which is highly conserved between humans, mouse and rat (positions 4060 to 4219) is shown in italics and in bold (cf. also FIG. 2).

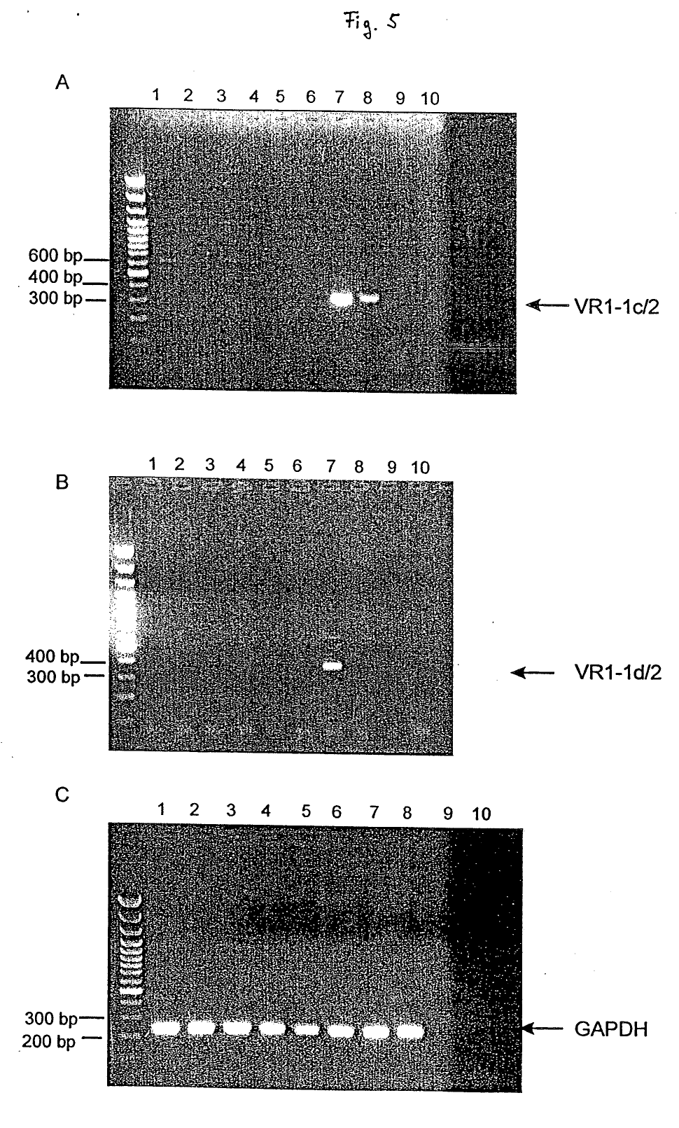

FIG. 5 shows photographs of 1.5% agarose gels of RT-PCR probes which served for amplification of exon 1c/2 and exon 1d/2 fragments of the VR1 mRNA in various tissues of the rat. 10 μl of the PCR reactions carried out with the primer pairs 1C-145F/1c417R (A) and VR1d-18F/1c-417R (B) or with GAPDH primers (C) and cDNA from the brain (track 1), heart (track 2), liver (track 3), intestine (track 4), spleen (track 5), kidney (track 6), spinal ganglia (track 7) and muscle (track 8) were separated. The reactions with the GAPDH primers served as a positive control. 1 μl of the particular cDNA solution was employed in the PCR reactions. For a further control, RNA was taken from all the tissue isolates used and was tested in a PCR with the various primer pairs (track 9). A further control was carried out without cDNA or RNA solution (track 10). The expected size of the products is 292 bp (A), 364 bp (B) and 227 bp (C). A further fragment with a length of about 600 bp is to be seen in track 1 in FIG. 5A. The larger fragment in track 7 of FIG. 5B is possibly a PCR artifact.

FIG. 6 is a diagram of the 5′ ends of the various human VR1 cDNAs. The genomic DNA section on which exons 1a, 1b, 1c, 1d and 2 are located is shown in the upper part of the figure. The 5′ regions with exon 1 and 2 of the various cDNA forms are moreover outlined. The Accession Numbers of the sequences are shown at the side.

The following embodiment examples explain the present invention in more detail, without limiting it.

### EXAMPLE 1

Identification of the 5′ Ends of the VR1 mRNA of the Rat

The 5′ ends of the VR1 mRNA of the rat were isolated from spinal ganglia mRNA with the aid of the 5′ RACE (5′ rapid amplification of the cDNA ends) method (RACE-PCR Kit, Clontech). The oligonucleotides AGW85 and rVR72 (5′-CCTCTGAGTCTAAGCTAGCCCGTTGTT-3′,5′-TAGCCCGTTGTTCCATCCTTTCCAG-3′) were used as gene-specific primers. Both primers hybridize in exon 2 of the VR1 cDNA sequence of the rat with the GenBank Accession Number AF029310 (position 85-111 and 72-96). The sequence AF029310 is also deposited in GenBank with the designation VR1L1 under Accession Number AB040873. 3 different types of RACE-PCR fragments which differ in their 5′ sequence were isolated. All the fragments contain 49 nucleotides of exon 2 in the 3′ region (see FIG. 1). The 5′ sequences of the fragments were identified in the genomic sequence of the rat with the GenBank Accession Number AC126839 with the aid of the FASTA and BLAST computer programs, the sequence in FIG. 1A being divided into two sections and therefore comprising two exons (95 bp and 168 bp). On the basis of the position in the sequence AC126839, the 5′ sequences are called exon 1a and 1b (position 53696-53790 and 71745-71912; FIG. 1A), exon 1c (position 87717-87907; FIG. 1B) and exon 1d (position 88077-88214; FIG. 1C) in the following. The sequence in FIG. 1B contains the first 47 nucleotides of exon 1 of the cDNA AF029310. Fragments with different start points were isolated from the exon 1c type. The 1c exon sequences of various sizes comprise 191, 115, 103, 79, 76, 46 and 38 bp.

### EXAMPLE 2

The Human VR1 Gene Contains 4 Different Exon 1 Variants

The work by Quing Xue et al. (2001, Genomics 76: 14 bis 20) describes the gene structure of the human VR1 gene and shows the location of exons 1a, 1b and 1c on the genomic DNA.

On the basis of the bioinformatic analyses of human VR1 sequences deposited in GenBank, a further exon 1 was identified in humans (FIG. 6). The section designated exon 1d is located on the genomic DNA downstream of exon 1c. Sequence comparison of the VR1 cDNAs showed that the transcripts differ only in the sequence of exon 1.

### EXAMPLE 3

Isolation of Genomic VR1 Sequences of the Rat

Genomic DNA was isolated with the aid of the GenomeWalker Kit from Clontech. This reaction system contains four different fractions of genomic DNA fragments. Each fraction was digested with a different restriction enzyme (EcoR V, Dra I, Pvu II, Ssp I) and the DNA fragments formed were coupled with a DNA adapter. The DNA adapter contains the sequences of primers AP1 and AP2. For isolation of the sequences, the genomic DNA was amplified by means of a Nested PCR. The primers AP1 and AP2 and two gene-specific primers were used for this. A fragment 1,450 bp in size in the 5′-upstream region of exon 1a was concentrated with the aid of the primers VR1ab-35R (5′-CGAGAGTGACGGGTCGCGAAGTCAT-3′) and VR1ab-1R (5′-GACAGCACAACTCAGGCGGCTTGAA-3′) and contains the first 27 nucleotides of the RACE fragment 1ab (FIG. 3 (SEQ ID NO: 7)). Starting from the published rat cDNA (GenBank Accession Number AF029310), two overlapping fragments were amplified from the region 5′-upstream of exon 1c by means of two Nested PCRs. The sequence comprises a total of 4,361 bp and contains the first 172 nucleotides of the RACE fragment 1c (FIG. 4 (SEQ ID NO: 8)). The first PCR was carried out with the primers AGW23 (5′-CAGCTAGGTGCAGGCACACCCCAAA-3′) and AGW4 (5′-CCCAAATGGAGCAAGTGCCTTGGAG-3′). The primers AGWZ021 (5′-TGTGAGCGCATGTGCCTATGCTTGCATT-3′) and AGWZ001 (5′-CTTGCATTTGCCAGACCCAGAGCAGGAT-3′) were used in the second PCR. The PCR fragments were ligated into the vector pGEM-T and sequenced. The sequences of the genomic fragments are shown in FIG. 3 (SEQ ID NO: 7) and 4. Furthermore, the sequences were identified in the genomic sequence of the rat with the databank number AC126839 with the BLAST computer program [position 52273-53695 (sequence in the 5′ region upstream of exon 1a) and position 83528-87716 (sequence in the 5′ region upstream of exon 1c); the data relate to sequences which contain no nucleotides of the RACE fragments].

### EXAMPLE 4

Identification of Orthologous Sequences of Exons 1a, 1b, 1c and 1d and of the Genomic DNA 5′-Upstream of Exons 1a and 1d

The VR1 sequences deposited in GenBank were searched in respect of orthologous sequences in the mouse and in humans with the aid of the BLAST and FASTA computer programs.

1. Exon 1a and 1b

In the mouse, the exons were identified in the sequence with the databank number AL663116 [position 1308-1401 (exon 1a, 92%) and position 19656-19823 (exon 1b, 94%)]. Exon 1a is also contained in the sequence with the databank number AL670399 [position 223345-223438 (92%)]. This sequence ends upstream before exon 1b, but contains a larger region 5′-upstream of exon 1a.

The cDNAs or ESTs of the mouse which contain the VR1 exons 1a and 1b and further sequence sections of the VR1 gene are not deposited in the relevant databanks. Nevertheless, the exons were identified in the cDNA of the gene carbohydrate kinase-like (CARKL) with the databank number NM_{—}029031 [position 26-119 (exon 1a; 92%) and position 911-1078 (exon 1 b; 94%)].

In humans, only the sequence of exon 1 b of the rat was identified. The human genomic sequence AF168787 shows a significant agreement (86%) with exon 1b of the rat in the section from position 50823 up to position 50656. Exon 1b of the rat, but not exon 1a, showed a homology to human CARKL-cDNA [NM_{—}013276, position 960-1127, 86%]. Human ESTs which, however, apart from exon 1b contain the sequence of the CARKL gene were also identified. The human exons 1a and 1b (XM_{—}040678/AL136801, position 1-242) are likewise contained in the CARKL cDNA sequence [NM_{—}013276, position 2689-2791 (exon 1a) and position 3535-3673 (exon 1b)]. No agreement between the cDNAs of the genes CARKL and VR1 was identified in other sequence sections. The sequences of the human exons 1a and 1b showed no homologies at all with exons 1a and 1b of the rat or with other sequences of the rat or mouse. The human genomic sequence with the databank number AF168787 contains the human exons 1a [position 44730-44628] and 1b [position 43884-43746].

2. Exon 1c

Exon 1c is contained in the genome sequence of the mouse with the GenBank Accession Number AL663116 [position 36005-36191, 96%]. Three EST/cDNA sequences 628 bp and 629 bp in size, the 5′ region of which shows agreement with exon 1c of the rat, are deposited in GenBank [BB656502, XM_{—}147517, XM_{—}112546; position 1-74, 98%]. These ESTs show a clear agreement with the VR1 cDNA of the rat [identical nucleotides 554/601 (92%)]. Human exon 1c shows no clear homology to exon 1c of the rat.

3. Exon 1 d

The sequence of exon 1d of the rat showed a significant agreement to the genomic sequence of the mouse in the first 114 nucleotides [AL663116, position 36360-36474; 86%]. Neither cDNAs nor ESTs of the mouse with the sequence of exon 1d are deposited in GenBank. No homology to human sequences was identified. Human exon 1d shows no agreement with exon 1d of the rat.

4. Sequences in the 5′ Direction of Exon 1a and Exon 1c or 1d

The genomic fragment of the rat 1,423 bp in size from the 5′ region of exon 1a is homologous in two sections, which are separated from one another by only 23 nucleotides, to the genomic sequence of the mouse [GenBank Accession Number AL670399; position 221931-222726 (80%) and 222754-223320 (86%)]. The region in the 5′ direction of exon 1d of the rat shows a clear agreement with the mouse sequence under the GenBank Accession Number AL663116 [position 32368-33403 (81%), 35013-35101 (90%), 35211-35264 (94%) and position 35290-36359 (87%)].

Corresponding sections in the 5′ direction of exons 1a and 1c or 1d of humans showed no significant agreement with the rat sequence. The only exception is a region 165 bp in size in the human sequence under the GenBank Accession Number AF168787 [position 32580-32416, (84%)]. This sequence section is conserved to a high degree between humans, mouse and rat (FIG. 2).

### EXAMPLE 5

Identification of DNA Binding Sites for Transcription Factors in the 5′ Region of Exons 1a and 1d of the VR1 Gene

Regulation of a gene at the transcription level takes place via regulatory regions (e.g. promoters, enhancers, silencers) which are built up in modular form from short regulatory sequence elements. These serve as binding sites for functional classes of proteins which are called transcription factors.

For identification of DNA motifs for transcription factors, the genomic sequences in the region in the 5′ direction of the VR1 exons 1a and 1d of the rat [AC126839; position 52273-53695 and 83528-88215], the mouse [AL670399; position 221931-223344; AL663116, position 31673-36359] and humans [AF168787, 44731-43231 and 36616-33151] were analysed in respect of possible DNA binding sites for transcription factors with the aid of the MatInspector computer program (sense Strand, Core Simil.: 1.000/Matrix Simil.: 0.900, Tab. 1 to 6 in the appendix). The figures of the genome sequences of the rat in the region of exon 1a and 1c/1d (FIG. 3 (SEQ ID NO: 7) and 4) show DNA motifs for transcription factors which are located on the sense DNA strand and also at the same position in the sequence of the mouse. In the sequence in the 5′ direction of exon 1a (FIG. 3 (SEQ ID NO: 7)), these are the binding motifs for the transcription factors MZF1 (myeloid zinc finger protein 1; position 39, 173, 1169), NFkappaB (nuclear factor-kappaB; position 39), GATA 1/2/3 (GATA-binding factor; position 62, 376, 1076), IK 2 and Klf 7 (Ikaros factor 2 and Krüppel-like factor 7; position 174, 517, 1087, 1235), NFAT (nuclear factor of activated T-cells; position 176, 1089), AP4 (activator protein 4; position 336), SRY (sex-determining region Y gene product; position 392), SOX5 (Sox-5; position 393), CP2 (position 498), cMyb (position 824), SREBP1 (sterol regulatory element-binding protein; position 982), deltaEF1 (delta-crystalline/E2-box factor 1; position 984, 998, 1118, 1294), MyoD (myoblast determining factor; position 983, 997), GKLF (gut-enriched Krüppel-like factor; position 1099), NRF2 (nuclear respiratory factor 2; position 1104), NF1 (nuclear factor 1; position 1122), CETS1P54 (c-Ets (p54); position 1254) and NFY (nuclear factor Y; position 1346). In the sequence in the 5′ direction of exon 1d (FIG. 4 (SEQ ID NO: 8)), binding sites for the following transcription factors were identified: TH1E47 (Thing1/E47 heterodimer; position 560, 1533), RORA1 (RAR-related orphan receptor alpha1; position 699), SRY (position 744), GFI1 (growth factor independence 1; position 749), AP1 (activator protein 1; position 870, 998), deltaEF1 (position 1030, 4372), GATA 1 (position 1129), TCF11 (TCF11/KCR-F1/Nrf1 homodimers; position 1381), MZF1 position 3375, 4255), IK2/1 and Klf 7 (position 3376, 4137, 4149, 4159, 4505), Brn2 (POU factor Brn2; position 3484), cMyb (position 3557), S8 (position 3731), MyoD (position 3890), NFAT (position 4013, 4139), NKX25 (homeodomain factor Nkx-2.5/Csx; position 4065), NF1 (position 4104), AP4 (position 4179, 4182, 4308, 4334, 4418), HNF3B (hepatocyte nuclear factor-3beta; position 4204) and HFH2 (HNF3 forkhead homologue 2; position 4204). Since a significant agreement between the sequences of humans and of the rat or the mouse exists only in a section 165 bp in size in the region of exon 1c, the sequence positions of DNA binding sites in the human VR1 sequence have not been taken into account in FIGS. 3 and 4. Nevertheless, almost all the DNA motifs which are at the same sequence position in the rat and mouse were identified in the corresponding sections in the 5′ direction of the human exons 1a and 1c/1d. Exceptions are the binding sites of the factors cMyb, GATA 1/3/2, GKLF, NFY, NRF2, SOX5, SREBP1 and SRY, which were not identified in the region 1.5 kb in size (sense strand) in the 5′ direction of human exon 1a.

In addition to the activating function of the transcription factors listed, the repressor action of the factors delta EF1 and GFI1 is known in particular (Funahasi et al. (1993) Development 119 (2): 433-446; Zweidler et al. (1996) Molecular and Cellular Biology, August: 4024-4034).

### EXAMPLE 6

Expression of Transcript Variants of the VR1 Gene

RT-PCR experiments were carried out with forward primers which hybridize specifically in exon 1c (1C-145F; 5′-CAGCTCCAAGGCACTTGCTC-3′) and exon 1d (VR1d-18F; 5′-GAGAGGTGGTGGTCAGTTGGCTTATGT-3′). The primer 1c-417R (5′-GCCAGCCCGCCTTCCTCATA-3′), which is specific for exon 2, was used as a reverse primer. RT-PCRs were carried out with GAPDH primers (5′-CGACCCCTTCATTGACCTCAACTACATG-3′ and 5′-CCCCGGCCTTCTCCATGGTGGTGAAGAC-3′) as a control. Total RNA was isolated from the brain, heart, liver, intestine, spleen, kidney, spinal ganglia and muscle of the rat, treated with DNase I and transcribed into cDNA. 2.5 μg of total RNA were used for the reverse transcription. The reaction batch was topped up to a final volume of 50 μl. 1 μl of the particular cDNA solution was employed for a 50 μl PCR reaction batch. The size of the PCR products was expected as follows: 292 bp (1C-145F/1c-417R), 364 bp (VR1d-18F/1c-417R) and 227 bp (GAPDH primer).

The RT-PCR experiments show that the VR1 variant which contains exon 1c is synthesized in the spinal ganglia and in the muscle (FIG. 5A). In contrast, the mRNA with exon 1d was detected only in spinal ganglia (FIG. 5B). Starting from exon 1c, using brain cDNA a PCR fragment which had approx. twice the length of the expected size and probably originates from a further variant of the VR1 mRNA was generated.

These results indicate a tissue- and therefore cell-specific expression of VR1 transcripts which differ in respect of exon 1. Accordingly, the VR1 gene is activated specifically from different promoters in the various tissue types or cells.

### SUMMARY

The present invention shows that four exon 1 variants exist both in humans and in the rat. On the basis of the location on the genomic DNA, the exons are called 1a, 1b, 1c and 1d. Three different transcript types were identified in the rat. The first variant contains exons 1a and 1b, the second exon 1c and the third exon 1d. Analysis of human VR1 cDNAs shows that these transcript forms also exist in humans. On the basis of the significant agreement in the sequences between the rat and mouse, the existence of this VR1 gene structure is also probable in the mouse.

It was furthermore possible to demonstrate that the transcript variants of the VR1 gene are expressed differently in various tissue types. Accordingly, the VR1 gene is activated from different promoters in the various tissue or cells types.

Binding sites for transcription factors which function as activators or repressors were identified in the 5′ regions of exons 1a, 1c and 1d by bioinformatic analyses of the corresponding sequences in the rat, the mouse and in humans.

The VR1 variant which contains exon 1c was detected in muscle tissue, while the VR1 transcript with exon 1d was not detected in the muscle. The different expression profile of the VR1 variants and the identification of DNA binding sites for transcription factors lead to the conclusion that different combinations of transcription factors bind in the 5′ regions upstream of exons 1a, 1c and 1d and thereby effect a tissue- and/or cell-specific expression of the various VR1 variants.

The present results show that the various 5′ regions of exons 1a, 1c and 1d and the binding sites for transcription factors contained therein play an important role in the tissue- and cell-specific regulation of the expression of the VR1 gene. This form of gene structure in combination with the DNA motifs for various transcription factors moreover renders possible an inducibility of the VR1 gene in the well-differentiated organism

Appendix: Tables 1 to 6

(Where descending sequence regions are given, the sequences deposited under the corresponding GenBank Accession Numbers are reverse sequences.)

Tab. 1: Transcription factor binding sites in the 5′ region upstream of the VR1 exon 1a of the rat.

The sequence of the rat (GenomeWalker clone GW-3; AC126839, position 52273-53695) was analysed in respect of possible DNA binding sites for transcription factors with the aid of the MatInspector computer program (sense Strand, Core Simil.: 1.000, Matrix Simil.: 0.900). The transcription factors which were identified at the same position as in the mouse sequence are provided in bold face in the table.

TABLE 2

Transcription factor binding sites in the 5′ region
upstream of the VR1 exon 1a of the mouse.

| Matrix | Position(str) | Core | Matrix | |
|---|---|---|---|---|
| Name | of Matrix | Simil. | Simil. | Sequence |
| V$SRY 02 | 20 (+) | 1.000 | 0.910 | gccaACAAtcca |
| V$SOX5 01 | 21 (+) | 1.000 | 0.983 | ccaaCAATcc |
| V$MZF1 01 | 36 (+) | 1.000 | 1.000 | agtGGGGa |
| Y$NFKAPPAB50 01 | 39 (+) | 1.000 | 0.904 | GGGGagaccc |
| V$IK2 01 | 56 (+) | 1.000 | 0.918 | tcaaGGGAtaac |
| V$GATA1 05 | 59 (+) | 1.000 | 0.945 | aggGATAaca |
| V$GATA3 02 | 59 (+) | 1.000 | 0.949 | aggGATAaca |
| V$GATA2 02 | 59 (+) | 1.000 | 0.944 | aggGATAaca |
| V$LMO2COM 02 | 60 (+) | 1.000 | 0.905 | ggGATAaca |
| V$HNF3B 01 | 143 (+) | 1.000 | 0.936 | tggaaTATTtattaa |
| V$IK2 01 | 170 (+) | 1.000 | 0.925 | aatgGCGAaaca |
| V$MZF1 01 | 170 (+) | 1.000 | 0.979 | aatGGGGa |
| V$NFAT Q6 | 171 (+) | 1.000 | 0.923 | atgggGAAAcag |
| V$S8 01 | 192 (+) | 1.000 | 0.939 | cccacagaATTAaagt |
| V$TST1 01 | 195 (+) | 1.000 | 0.927 | acagAATTaaagtta |
| V$TH1E47 01 | 248 (+) | 1.000 | 0.914 | aataggttCTGGatgt |
| V$S8 01 | 307 (+) | 1.000 | 0.964 | acgccataATTAaaaa |
| V$NKX25 02 | 311 (+) | 1.000 | 0.951 | caTAATta |
| V$AP4 Q5 | 334 (+) | 1.000 | 0.947 | acCAGCtgta |
| V$GATA1 06 | 361 (+) | 1.000 | 0.946 | attGATAaga |
| V$GATA2 02 | 361 (+) | 1.000 | 0.977 | attGATAaga |
| V$GATA3 02 | 361 (+) | 1.000 | 0.933 | attGATAaga |
| V$LMO2COM 02 | 362 (+) | 1.000 | 0.928 | ttGATAaga |
| V$EVI1 05 | 363 (+) | 1.000 | 0.959 | tgataaGATAa |
| V$EVI1 03 | 363 (+) | 1.000 | 0.945 | tgataAGATaa |
| V$EVI1 02 | 363 (+) | 1.000 | 0.986 | tgatAAGAtaa |
| V$GATA1 04 | 365 (+) | 1.000 | 0.963 | ataaGATAaaaga |
| V$GATA2 02 | 366 (+) | 1.000 | 0.955 | taaGATAaaa |
| V$GATA3 02 | 366 (+) | 1.000 | 0.956 | taaGATAaaa |
| V$LMO2COM 02 | 367 (+) | 1.000 | 0.916 | aaGATAaaa |
| V$GATA1 06 | 373 (+) | 1.000 | 0.980 | aaaGATAaaa |
| V$GATA2 02 | 373 (+) | 1.000 | 0.970 | aaaGATAaaa |
| V$GATA3 02 | 373 (+) | 1.000 | 0.972 | aaaGATAaaa |
| V$LMO2COM 02 | 374 (+) | 1.000 | 0.916 | aaGATAaaa |
| V$SRY 02 | 388 (+) | 1.000 | 0.952 | aaaaACAAtgat |
| V$SOX5 01 | 389 (+) | 1.000 | 0.986 | aaaaCAATga |
| V$CEBPB 01 | 393 (+) | 1.000 | 0.930 | caatgatGCAAtca |
| V$GFI1 01 | 394 (+) | 1.000 | 0.937 | aatgatgcAATCaatgttatttat |
| V$GATA1 02 | 457 (+) | 1.000 | 0.935 | gcataGATAgtcat |
| V$LMO2COM 02 | 460 (+) | 1.000 | 0.937 | taGATAgtc |
| V$TCF11 01 | 466 (+) | 1.000 | 0.973 | GTCAttcctcaac |
| V$CP2 01 | 491 (+) | 1.000 | 0.966 | gcaagacCCAG |
| V$IK2 01 | 513 (+) | 1.000 | 0.920 | ttctGGGAgaat |
| V$AP4 Q5 | 526 (+) | 1.000 | 0.920 | aaCAGCtcct |
| V$NFY Q6 | 692 (+) | 1.000 | 0.906 | tcaCCAAtact |
| V$IK1 01 | 714 (+) | 1.000 | 0.942 | acctGGGAatgtg |
| V$IK2 01 | 714 (+) | 1.000 | 0.950 | acctGGGAatgt |
| Y$CMYB 01 | 814 (+) | 1.000 | 0.940 | gctcatgtctcTTGgggt |
| V$GATA1 02 | 910 (+) | 1.000 | 0.922 | cattaGATAccgct |
| V$LMO2COM 02 | 913 (+) | 1.000 | 0.977 | taGATAccg |
| V$AP1 Q2 | 952 (+) | 1.000 | 0.948 | gCTGACtttgt |
| V$CMYB 01 | 968 (+) | 1.000 | 0.909 | agcggggccaGTTGtcac |
| V$SREBP1 01 | 980 (+) | 1.000 | 0.902 | tgTCACctgac |
| V$DELTAEF1 01 | 980 (+) | 1.000 | 0.979 | tgtcACCTgac |
| V$MYOD Q6 | 981 (+) | 1.000 | 0.972 | gtCACCtgac |
| V$DELTAEF1 01 | 994 (+) | 1.000 | 0.976 | aaccACCTgac |
| V$MYOD Q6 | 995 (+) | 1.000 | 0.989 | acCACCtgac |
| V$GFI1 01 | 1047 (+) | 1.000 | 0.901 | ttttcaagAATCatcggcagctaa |
| V$GATA1 02 | 1071 (+) | 1.000 | 0.966 | cacttGATAgggtt |
| V$LMO2COM 02 | 1074 (+) | 1.000 | 0.972 | ttGATAggg |
| V$IK1 01 | 1083 (+) | 1.000 | 0.910 | ttgtGGGAaaggt |
| V$IK2 01 | 1083 (+) | 1.000 | 0.953 | ttgtGGGAaagg |
| V$NFAT Q6 | 1084 (+) | 1.000 | 0.912 | tgtggGAAAggt |
| V$GKLF 01 | 1089 (+) | 1.000 | 0.906 | gaaaggtggaAGGG |
| V$NRF2 01 | 1101 (+) | 1.000 | 0.914 | ggcGGAAgag |
| V$NF1 Q6 | 1111 (+) | 1.000 | 0.949 | cttTGGCaccttggcaag |
| V$DELTAEF1 01 | 1114 (+) | 1.000 | 0.947 | tggcACCTtgg |
| V$NF1 Q6 | 1119 (+) | 1.000 | 0.935 | cctTGGCaaggacagggg |
| V$MZF1 01 | 1145 (+) | 1.000 | 0.975 | tgaGGGGa |
| V$MZF1 01 | 1166 (+) | 1.000 | 0.957 | gtaGGGGa |
| V$IK2 01 | 1231 (+) | 1.000 | 0.938 | ccctGGGAcgcc |
| V$CETS1P54 01 | 1252 (+) | 1.000 | 0.919 | ccCGGAactt |
| V$DELTAEF1 01 | 1290 (+) | 1.000 | 0.948 | ctccACCTatc |
| V$NFY 01 | 1341 (+) | 1.000 | 0.949 | ctcgaCCAAtaggagc |
| V$NF1 Q6 | 1365 (+) | 1.000 | 0.946 | gatTGGCagggacgcccc |
| V$IK2 01 | 1369 (+) | 1.000 | 0.908 | ggcaGGGAcgcc |

The sequence of the mouse (AL670339, position 221931-223344) was analysed in respect of possible DNA binding sites for transcription factors with the aid of the MatInspector computer program (sense Strand, Core Simil.: 1.000, Matrix Simil.: 0.900). The transcription factors which were identified at the same position as in the sequence of the rat are provided in boldface in the table.

TABLE 3

Transcription factor binding sites in the 5′
region upstream of the VR1 exon 1a of humans.

| | Posi- | | | |
|---|---|---|---|---|
| | tion | | | |
| | (str) | | | |
| Matrix | of | Core | Matrix | |
| Name | Matrix | Simil. | Simil. | Sequence |
| V$NKX25 02 | 11 (+) | 1.000 | 0.939 | ccTAATtg |
| V$NF1 Q6 | 14 (+) | 1.000 | 0.968 | aatTGGCcataatccatg |
| V$MZF1 01 | 34 (+) | 1.000 | 1.000 | agtGGGGa |
| V$NFKAPPAB50 | 37 (+) | 1.000 | 0.904 | GGGGagaccc |
| 01 | | | | |
| V$GATA1 02 | 55 (+) | 1.000 | 0.925 | caaggGATAgtaca |
| V$TH1E47 01 | 132 | 1.000 | 0.916 | gtatgattCTGGaata |
| | (+) | | | |
| V$NKX25 02 | 165 | 1.000 | 0.903 | ctTAATgg |
| | (+) | | | |
| V$IK2 01 | 168 | 1.000 | 0.925 | aatgcGGAaaca |
| | (+) | | | |
| V$MZF1 01 | 168 | 1.000 | 0.979 | aatGGGGa |
| | (+) | | | |
| V$NPAT Q6 | 169 | 1.000 | 0.923 | atgggGAAAcag |
| | (+) | | | |
| V$TCF11 01 | 309 | 1.000 | 0.980 | GTCAtaataaaaa |
| | (+) | | | |
| V$AP4 Q5 | 334 | 1.000 | 0.947 | acCAGCtgta |
| | (+) | | | |
| V$BARBIE 01 | 343 | 1.000 | 0.926 | attaAAAGtttgagg |
| | (+) | | | |
| V$GATA1 05 | 366 | 1.000 | 0.967 | taaGATAaca |
| | (+) | | | |
| V$GATA2 02 | 366 | 1.000 | 0.965 | taaGATAaca |
| | (+) | | | |
| V$GATA3 02 | 366 | 1.000 | 0.957 | taaGATAaca |
| | (+) | | | |
| V$LMO2COM 02 | 367 | 1.000 | 0.938 | aaGATAaca |
| | (+) | | | |
| V$SRY 02 | 369 | 1.000 | 0.944 | gataACAAaaag |
| | (+) | | | |
| V$SRY 02 | 384 | 1.000 | 0.947 | agaaACAAtgag |
| | (+) | | | |
| V$SOX5 01 | 385 | 1.000 | 0.985 | gaaaCAATga |
| | (+) | | | |
| V$NFH2 01 | 450 | 1.000 | 0.936 | gatTGTTatttt |
| | (+) | | | |
| V$CP2 01 | 482 | 1.000 | 0.935 | gcaagatCCAG |
| | (+) | | | |
| V$IK2 01 | 506 | 1.000 | 0.925 | ctctGGGAgaat |
| | (+) | | | |
| V$AP4 Q5 | 632 | 1.000 | 0.947 | acCAGCtgtc |
| | (+) | | | |
| V$CMYB 01 | 777 | 1.000 | 0.945 | gctaatgtctGTTGgggt |
| | (+) | | | |
| V$PADS C | 796 | 1.000 | 0.932 | gGTGGTgtc |
| | (+) | | | |
| V$TCF11 01 | 802 | 1.000 | 0.968 | GTCAtaccagaaa |
| | (+) | | | |
| V$DELTAEF1 01 | 843 | 1.000 | 0.994 | tctcACCTgac |
| | (+) | | | |
| V$MYOD Q6 | 844 | 1.000 | 0.970 | ctCACCtgac |
| | (+) | | | |
| V$AP1FJ Q2 | 848 | 1.000 | 0.906 | ccTGACagctt |
| | (+) | | | |
| V$SOX5 01 | 889 | 1.000 | 0.978 | gcaaCAATct |
| | (+) | | | |
| V$AP4 Q5 | 964 | 1.000 | 0.947 | gcCAGCtgtc |
| | (+) | | | |
| V$SREBP1 01 | 970 | 1.000 | 0.902 | tgTCACctgac |
| | (+) | | | |
| V$DELTAEF1 01 | 970 | 1.000 | 0.979 | tgtcACCTgac |
| | (+) | | | |
| V$MYOD Q6 | 971 | 1.000 | 0.972 | gtCACCtgac |
| | (+) | | | |
| V$DELTAEF1 01 | 984 | 1.000 | 0.976 | aaccACCTgac |
| | (+) | | | |
| V$MYOD Q6 | 985 | 1.000 | 0.989 | acCACCtgac |
| | (+) | | | |
| V$AP1FJ Q2 | 989 | 1.000 | 0.907 | ccTGACttgaa |
| | (+) | | | |
| V$GATA1 03 | 1050 | 1.000 | 0.955 | aggagGATAacgct |
| | (+) | | | |
| V$GATA2 02 | 1052 | 1.000 | 0.913 | gagGATAacg |
| | (+) | | | |
| V$LMO2COM 02 | 1053 | 1.000 | 0.947 | agGATAacg |
| | (+) | | | |
| V$IK2 01 | 1072 | 1.000 | 0.922 | gtgaGGGAaggg |
| | (+) | | | |
| V$GKLF 01 | 1078 | 1.000 | 0.903 | gaagggtggaAGGG |
| | (+) | | | |
| V$NRF2 01 | 1090 | 1.000 | 0.914 | ggcGGAAgag |
| | (+) | | | |
| V$DELTAEF1 01 | 1103 | 1.000 | 0.947 | aggcACCTtgg |
| | (+) | | | |
| V$NF1 Q6 | 1108 | 1.000 | 0.934 | cctTGGCagggacagggg |
| | (+) | | | |
| V$MZF1 01 | 1155 | 1.000 | 0.957 | gtaGGGGa |
| | (+) | | | |
| V$IK2 01 | 1155 | 1.000 | 0.910 | gtagGGGAagca |
| | (+) | | | |
| V$IK2 01 | 1220 | 1.000 | 0.939 | ccctGGGAcccc |
| | (+) | | | |
| V$CETS1PS4 01 | 1241 | 1.000 | 0.907 | ccCGGAacct |
| | (+) | | | |
| V$AP1FJ Q2 | 1250 | 1.000 | 0.914 | tcTGACcaata |
| | (+) | | | |
| V$NFY Q6 | 1252 | 1.000 | 0.931 | tgaCCAAtaga |
| | (+) | | | |
| V$CDPCR3HD 01 | 1257 | 1.000 | 0.973 | aataGATCcc |
| | (+) | | | |
| V$DELTAEF1 01 | 1281 | 1.000 | 0.948 | ctccACCTatc |
| | (+) | | | |
| V$CETS1P54 01 | 1292 | 1.000 | 0.944 | acCGGAggcc |
| | (+) | | | |
| V$MZF1 01 | 1304 | 1.000 | 0.989 | tgtGGGGa |
| | (+) | | | |
| V$AHRARNT 01 | 1306 | 1.000 | 0.902 | tggggagcgCGTGgtg |
| | (+) | | | |
| V$PADS C | 1315 | 1.000 | 0.911 | CGTGGTgtt |
| | (+) | | | |
| V$HNF3B 01 | 1315 | 1.000 | 0.925 | cgtggTGTTtgcttt |
| | (+) | | | |
| V$HFH3 01 | 1317 | 1.000 | 0.917 | tggTGTTtgcttt |
| | (+) | | | |
| V$NFY 01 | 1331 | 1.000 | 0.923 | ctcgaCCAAtagaagt |
| | (+) | | | |
| V$NKX25 01 | 1395 | 1.000 | 0.938 | tgAAGTg |
| | (+) | | | |

The sequence of humans (AF168787, position 44731-43231) was analysed in respect of possible DNA binding sites for transcription factors with the aid of the MatInspector computer program (sense Strand, Core Simil.: 1.000, Matrix Simil.: 0.900). The transcription factors which were located at the same positions in the sequence of the rat and the mouse and were identified in the human sequence in the corresponding sequence section although not necessarily at the same position are provided in boldface. The factors cMyb, GATA 1/2/3, GKLF, NFY, NRF2, SOX5, SREBP1 and SRY were not identified in the sense DNA strand 5′-upstream of human exon 1a.

TABLE 4

Transcription factor binding sites in the 5′ region
upstream of the VR1 exon 1d of the rat.

| Matrix | Position(str) | Core | Matrix | |
|---|---|---|---|---|
| Name | of Matrix | Simil. | Simil. | Sequence |
| V$DELTAEF1 01 | 5 (+) | 1.000 | 0.985 | acccACCTgac |
| V$MYOD Q6 | 6 (+) | 1.000 | 0.982 | ccCACCtgac |
| V$MZF1 01 | 32 (+) | 1.000 | 0.956 | ctgGGGGa |
| V$DELTAEF1 01 | 41 (+) | 1.000 | 0.943 | aggcACCTgcc |
| V$MYOD Q6 | 42 (+) | 1.000 | 0.990 | ggCACCtgcc |
| V$AP4 Q5 | 54 (+) | 1.000 | 0.953 | acCAGCtggc |
| V$ER Q6 | 58 (+) | 1.000 | 0.906 | gctggcctcagTGACcaga |
| V$AP1FJ Q2 | 67 (+) | 1.000 | 0.918 | agTGACcagaa |
| V$ARNT 01 | 99 (+) | 1.000 | 0.954 | tggggcaCGTGacccg |
| V$MAX 01 | 100 (+) | .000 | 0.942 | ggggCACGtgaccc |
| V$USF 01 | 100 (+) | 1.000 | 0.993 | ggggCACGtgaccc |
| V$NMYC 01 | 101 (+) | 1.000 | 0.961 | gggcaCGTGacc |
| V$MYCMAX 02 | 101 (+) | 1.000 | 0.902 | gggCACGtgacc |
| V$USF Q6 | 102 (+) | 1.000 | 0.953 | ggCACGtgac |
| V$USF C | 103 (+) | 1.000 | 0.991 | gCACGTga |
| V$AP1FJ Q2 | 106 (+) | 1.000 | 0.902 | cgTGACccggg |
| V$AP4 Q5 | 162 (+) | 1.000 | 0.907 | ttCAGCagct |
| V$AP4 Q5 | 165 (+) | 1.000 | 0.909 | agCAGCtcca |
| V$IK2 01 | 202 (+) | 1.000 | 0.945 | cagtGGGAgtgc |
| V$CREB 02 | 222 (+) | 1.000 | 0.922 | ggaaTGACgtgc |
| V$XBP1 01 | 222 (+) | 1.000 | 0.912 | ggaatgACGTgctgaag |
| V$CREB 01 | 226 (+) | 1.000 | 0.925 | TGACgtgc |
| V$CREL 01 | 251 (+) | 1.000 | 0.966 | agggctTTCC |
| V$NFKAPPAB65 01 | 251 (+) | 1.000 | 0.936 | agggctTTCC |
| V$E47 01 | 290 (+) | 1.000 | 0.920 | gatGCAGctgtcggg |
| V$AP4 Q5 | 292 (+) | 1.000 | 0.947 | tgCAGCtgtc |
| V$AP4 Q5 | 304 (+) | 1.000 | 0.920 | ggCAGCtctg |
| V$TCF11 01 | 314 (+) | 1.000 | 0.981 | GTCAtgctccgga |
| V$DELTAEF1 01 | 326 (+) | 1.000 | 0.955 | agacACCTcaa |
| V$AP1FJ Q2 | 405 (+) | 1.000 | 0.921 | ccTGACaccat |
| V$TCF11 01 | 422 (+) | 1.000 | 0.993 | GTCAtcctttccc |
| V$BRN2 01 | 543 (+) | 1.000 | 0.923 | cagatcccAAATgagt |
| V$AP1FJ Q2 | 569 (+) | 1.000 | 0.907 | atTGACccacc |
| V$DELTAEF1 01 | 573 (+) | 1.000 | 0.969 | acccACCTggg |
| V$MYOD Q6 | 574 (+) | 1.000 | 0.910 | ccCACCtggg |
| V$IK2 01 | 577 (+) | 1.000 | 0.915 | acctGGGAgcta |
| V$IK2 01 | 602 (+) | 1.000 | 0.929 | atgtGGGAgaga |
| V$AP4 Q5 | 647 (+) | 1.000 | 0.925 | gtCAGCaggc |
| V$DELTAEF1 01 | 666 (+) | 1.000 | 0.968 | gttcACCTgta |
| V$MYOD Q6 | 667 (+) | 1.000 | 0.914 | ttCACCtgta |
| V$NKX25 01 | 733 (+) | 1.000 | 0.932 | ccAAGTg |
| V$IK1 01 | 735 (+) | 1.000 | 0.909 | aagtGGGAaaaga |
| V$IK2 01 | 735 (+) | 1.000 | 0.958 | aagtGGGAaaag |
| V$NFAT Q6 | 736 (+) | 1.000 | 0.935 | agtggGAAAaga |
| V$NFAT Q6 | 818 (+) | 1.000 | 0.960 | ttctgGAAAagt |
| V$CETS1P54 01 | 856 (+) | 1.000 | 0.944 | acCGGAggcc |
| V$IK2 01 | 910 (+) | 1.000 | 0.914 | gccaGGGAttga |
| V$AP1FJ Q2 | 917 (+) | 1.000 | 0.903 | atTGACccaag |
| V$CP2 01 | 1012 (+) | 1.000 | 0.915 | gctgcacCCAG |
| V$MZF1 01 | 1096 (+) | 1.000 | 0.969 | aagGGGGa |
| V$IK2 01 | 1216 (+) | 1.000 | 0.937 | tcatGGGAaggg |
| V$IK2 01 | 1221 (+) | 1.000 | 0.906 | ggaaGGGAtgca |
| V$AHRARNT 01 | 1310 (+) | 1.000 | 0.902 | ttcgcttggCGTGggc |
| V$NF1 Q6 | 1313 (+) | 1.000 | 0.919 | gctTGGCgtgggctttgc |
| V$AP4 Q5 | 1352 (+) | 1.000 | 0.923 | ctCAGCagaa |
| V$NF1 Q6 | 1373 (+) | 1.000 | 0.913 | agtTGGCatccctgtagg |
| V$IK2 01 | 1385 (+) | 1.000 | 0.930 | tgtaGGGAtccc |
| V$IK2 01 | 1423 (+) | 1.000 | 0.937 | ggtaGGGAtggc |

The sequence of the rat (FIG. 4 (SEQ ID NO: 8). Position 1-4549; AC126839, position 83528-88215) was analysed in respect of possible DNA binding sites for transcription factors with the aid of the MatInspector computer program (sense Strand, Core Simil.: 1.000, Matrix Simil.: 0.900). The transcription factors which were identified at the same position as in the mouse sequence are provided in boldface in the table.

TABLE 5

Transcription factor binding sites in the 5′ region upstream
of the VR1 exon 1d of the mouse.

| | | | | |
|---|---|---|---|---|
| Matrix | Position(str) | Core | Matrix | |
| Name | of Matrix | Simil. | Simil. | Sequence |

| | | | | | |
|---|---|---|---|---|---|
| V$VBP 01 | 15 (+) | 1.000 | 0.921 | gTTACatata | |
| V$GFI1 01 | 38 (+) | 1.000 | 0.911 | ctttatcaAATCaaatgtgaatcc | |
| V$SRY 02 | 82 (+) | 1.000 | 0.915 | tctaACAAaacc | |
| V$OCT1 06 | 113 (+) | 1.000 | 0.903 | gatatttatATGCg | |
| V$NFAT Q6 | 134 (+) | 1.000 | 0.944 | attagGAAAcca | |
| V$NKX25 02 | 164 (+) | 1.000 | 0.951 | caTAATtc | |
| V$GFI1 01 | 203 (+) | 1.000 | 0.904 | gtatgcacAATCaaaacactgcag | |
| V$TCF11 01 | 268 (+) | 1.000 | 0.955 | GTCAttggcattg | |
| V$NF1 Q6 | 270 (+) | 1.000 | 0.915 | catTGGCattgtgtgctg | |
| V$NKX25 01 | 338 (+) | 1.000 | 0.938 | tgAAGTg | |
| V$CEBPB 01 | 345 (+) | 1.000 | 0.994 | aagttgtGCAAtgt | |
| V$DELTAEF1 01 | 384 (+) | 1.000 | 0.957 | aagcACCTcag | |
| V$AP4 Q5 | 390 (+) | 1.000 | 0.926 | ctCAGCtcca | |
| V$TCF11 01 | 492 (+) | 1.000 | 0.966 | GTCAtgtggagtt | |
| V$DELTAEF1 01 | 536 (+) | 1.000 | 0.954 | cagcACCTcag | homologous |
| V$TH1E47 01 | 552 (+) | 1.000 | 0.922 | cttggtttCTGGctgt | region ↓ |
| V$TCF11 01 | 568 (+) | 1.000 | 0.901 | GTCActatagcct | |
| V$AP4 Q5 | 596 (+) | 1.000 | 0.953 | gcCAGCtgtg | |
| V$STAT 01 | 615 (+) | 1.000 | 0.954 | TTCCtgtaa | |
| V$GATA1 03 | 652 (+) | 1.000 | 0.919 | aggttGATAcaagt | |
| V$RORA1 01 | 692 (+) | 1.000 | 0.947 | tgttccaGGTCag | |
| V$NF1 06 | 705 (+) | 1.000 | 0.925 | cctTGGCtatgtagcccg | |
| V$SRY 02 | 733 (+) | 1.000 | 0.914 | aaaaACAAcaaa | |
| V$SRY 02 | 740 (+) | 1.000 | 0.933 | acaaACAAaaat | |
| V$GFI1 01 | 741 (+) | 1.000 | 0.922 | caaacaaaAATCccagaggaactc | |
| V$IK2 01 | 839 (+) | 1.000 | 0.917 | gccaGGGAacag | |
| V$GATA1 02 | 854 (+) | 1.000 | 0.900 | ccctgGATAgcctt | |
| V$LMO2COM 02 | 857 (+) | 1.000 | 0.919 | tgGATAgcc | |
| V$AP1FJ Q2 | 868 (+) | 1.000 | 0.938 | agTGACctcta | |
| V$TCF11 01 | 905 (+) | 1.000 | 0.972 | GTCAtttgtcagt | |
| V$NF1 Q6 | 942 (+) | 1.000 | 0.933 | catTGGCcagagcttagg | |
| V$NFAT Q6 | 954 (+) | 1.000 | 0.976 | cttagGAAAacg | |
| V$TCF11 01 | 979 (+) | 1.000 | 0.967 | GTCAtcccgagag | |
| V$AP1FJ Q2 | 996 (+) | 1.000 | 0.903 | ctTGACttcca | |
| V$DELTAEF1 01 | 1026 (+) | 1.000 | 0.963 | catcACCTaga | |
| V$E47 02 | 1119 (+) | 1.000 | 0.930 | cagagCAGGtgatatt | |
| V$MYOD 01 | 1121 (+) | 1.000 | 0.927 | gagCAGGtgata | |
| V$LMO2COM 01 | 1121 (+) | 1.000 | 0.936 | gagCAGGtgata | |
| V$GATA1 04 | 1125 (+) | 1.000 | 0.904 | aggtGATAttcag | |
| V$AP4 Q5 | 1165 (+) | 1.000 | 0.953 | acCAGCtgct | |
| V$IK2 01 | 1198 (+) | 1.000 | 0.906 | cacaGGGAtggg | |
| V$MZF1 01 | 1204 (+) | 1.000 | 0.974 | gatGGGGa | |
| V$AP1FJ Q2 | 1226 (+) | 1.000 | 0.910 | atTGACccagg | |
| V$TCF11 01 | 1259 (+) | 1.000 | 0.977 | GTCAtgaaaacga | |
| V$E47 02 | 1329 (+) | 1.000 | 0.907 | ccatgCAGGtgatgtc | |
| V$MYOD 01 | 1331 (+) | 1.000 | 0.920 | atgCAGGtgatg | |
| V$LMO2COM 01 | 1331 (+) | 1.000 | 0.946 | atgCAGGtgatg | |
| V$HNF3B 01 | 1342 (+) | 1.000 | 0.905 | gtcttTGTTtccttt | |
| V$MZF1 01 | 1369 (+) | 1.000 | 0.974 | gatGGGGa | |
| V$IK2 01 | 1369 (+) | 1.000 | 0.903 | gatgGGGAcaga | |
| V$TCF11 01 | 1381 (+) | 1.000 | 0.954 | GTCAtacccagtg | |
| V$TATA 01 | 1469 (+) | 1.000 | 0.942 | ctaTAAAgagtcagg | |
| V$GATA1 04 | 1512 (+) | 1.000 | 0.914 | gcctGATAtcctg | |
| V$LMO2COM 02 | 1514 (+) | 1.000 | 0.932 | ctGATAtcc | homologous |
| V$TH1E47 01 | 1525 (+) | 1.000 | 0.939 | ctctgggtCTGGcaaa | region ↑ |
| V$AP4 Q5 | 1654 (+) | 1.000 | 0.938 | ctCAGCtcat | |
| V$CAAT 01 | 1674 (+) | 1.000 | 0.919 | tgagtCCAAtga | |
| V$NFY 01 | 1674 (+) | 1.000 | 0.929 | tgagtCCAAtgagata | |
| V$NFY Q6 | 1676 (+) | 1.000 | 0.914 | agtCCAAtgag | |
| V$GATA1 02 | 1681 (+) | 1.000 | 0.929 | aatgaGATAgtatg | |
| V$GATA2 03 | 1683 (+) | 1.000 | 0.930 | tgaGATAgta | |
| V$LMO2COM 02 | 1684 (+) | 1.000 | 0.925 | gaGATAgta | |
| V$GATA1 03 | 1701 (+) | 1.000 | 0.904 | gcgtaGATAccaac | |
| V$LMO2COM 02 | 1704 (+) | 1.000 | 0.934 | taGATAcca | |
| V$DELTAEF1 01 | 1765 (+) | 1.000 | 0.943 | cgccACCTgcc | |
| V$MYOD Q6 | 1766 (+) | 1.000 | 0.990 | gcCACCtgcc | |
| V$IK2 01 | 1847 (+) | 1.000 | 0.905 | gacaGGGAgggc | |
| V$NKX25 01 | 1928 (+) | 1.000 | 0.930 | gtAAGTg | |
| V$AP1FJ Q2 | 2033 (+) | 1.000 | 0.915 | gaTGACagaag | |
| V$NFAT Q6 | 2050 (+) | 1.000 | 0.965 | cccagGAAAaga | |
| V$NFAT Q6 | 2058 (+) | 1.000 | 0.952 | aagagGAAAtgc | |
| V$IK1 01 | 2098 (+) | 1.000 | 0.906 | gctgGGGAatctt | |
| V$IK2 01 | 2098 (+) | 1.000 | 0.917 | gctgGGGAatct | |
| V$MZF1 01 | 2098 (+) | 1.000 | 0.968 | gctGGGGa | |
| V$AP4 Q5 | 2110 (+) | 1.000 | 0.907 | ttCAGCagtg | |
| V$MZF1 01 | 2134 (+) | 1.000 | 0.968 | gctGGGGa | |
| V$IK2 01 | 2134 (+) | 1.000 | 0.918 | gctgGGGAagga | |
| V$IK1 01 | 2134 (+) | 1.000 | 0.900 | gctgGGGAaggac | |
| V$IK2 01 | 2168 (+) | 1.000 | 0.916 | agtaGGGAtgag | |
| V$GATA1 06 | 2196 (+) | 1.000 | 0.992 | ccaGATAaga | |
| V$GATA2 02 | 2196 (+) | 1.000 | 0.983 | ccaGATAaga | |
| V$GATA3 02 | 2196 (+) | 1.000 | 0.957 | ccaGATAaga | |
| V$LMO2COM 02 | 2197 (+) | 1.000 | 0.956 | caGATAaga | |
| V$EVI1 02 | 2198 (+) | 1.000 | 0.934 | agatAAGAgaa | |
| V$GKLF 01 | 2199 (+) | 1.000 | 0.922 | gataagagaaAGGG | |
| V$GKLF 01 | 2223 (+) | 1.000 | 0.923 | aaacagaaggAGGG | |
| V$IK2 01 | 2230 (+) | 1.000 | 0.900 | aggaGGGAtggg | |
| V$IK2 01 | 2235 (+) | 1.000 | 0.919 | ggatGGGAggga | |
| V$GATA1 04 | 2294 (+) | 1.000 | 0.944 | aagaGATAatatc | |
| V$GATA2 03 | 2295 (+) | 1.000 | 0.992 | agaGATAata | |
| V$GATA3 02 | 2295 (+) | 1.000 | 0.997 | agaGATAata | |
| V$LMO2COM 02 | 2296 (+) | 1.000 | 0.924 | gaGATAata | |
| V$MZF1 01 | 2340 (+) | 1.000 | 0.962 | ataGGGGa | |
| V$NKX25 02 | 2363 (+) | 1.000 | 0.971 | ctTAATtc | |
| V$GATA3 03 | 2373 (+) | 1.000 | 0.950 | acAGATcaga | |
| V$GATA1 02 | 2457 (+) | 1.000 | 0.941 | agagaGATAcaggg | |
| V$LMO2COM 02 | 2460 (+) | 1.000 | 0.956 | gaGATAcag | |
| V$IK2 01 | 2464 (+) | 1.000 | 0.914 | tacaGGGAagat | |
| V$GATA3 03 | 2470 (+) | 1.000 | 0.900 | gaAGATgata | |
| V$GATA1 03 | 2471 (+) | 1.000 | 0.968 | aagatGATAagaag | |
| V$GATA2 02 | 2473 (+) | 1.000 | 0.967 | gatGATAaga | |
| V$GATA3 02 | 2473 (+) | 1.000 | 0.930 | gatGATAaga | |
| V$GKLF 01 | 2473 (+) | 1.000 | 0.901 | gatgataagaAGGG | |
| V$LMO2COM 02 | 2474 (+) | 1.000 | 0.928 | atGATAaga | |
| V$CMYB 01 | 2503 (+) | 1.000 | 0.922 | agaagcagctGTTGggga | |
| V$E47 01 | 2504 (+) | 1.000 | 0.920 | gaaGCAGctgttggg | |
| V$AP4 Q5 | 2506 (+) | 1.000 | 0.976 | agCAGCtgtt | |
| V$IK2 01 | 2513 (+) | 1.000 | 0.908 | gttgGGGAcaaa | |
| V$MZF1 01 | 2513 (+) | 1.000 | 0.971 | gttGGGGa | |
| V$IK2 01 | 2560 (+) | 1.000 | 0.914 | gttgGGGAtttg | |
| V$MZF1 01 | 2560 (+) | 1.000 | 0.971 | gttGGGGa | |
| V$NF1 Q6 | 2567 (+) | 1.000 | 0.944 | attTGGCtcagtgacaga | |
| V$AP1FJ Q2 | 2576 (+) | 1.000 | 0.949 | agTGACagagc | |
| V$AP4 Q5 | 2622 (+) | 1.000 | 0.905 | ccCAGCtccg | |
| V$ISRE 01 | 2680 (+) | 1.000 | 0.918 | gaGTTTcagtttgcg | |
| V$VBP 01 | 2735 (+) | 1.000 | 0.945 | gTTACatgaa | |
| V$VMYB 01 | 2744 (+) | 1.000 | 0.936 | atgAACGgaa | |
| V$NRF2 01 | 2747 (+) | 1.000 | 0.931 | aacGGAAgat | |
| V$AP1FJ Q2 | 2754 (+) | 1.000 | 0.930 | gaTGACccaac | |
| V$GATA1 03 | 2780 (+) | 1.000 | 0.949 | gttagGATAaccag | |
| V$GATA2 02 | 2782 (+) | 1.000 | 0.902 | tagGATAacc | |
| V$LMO2COM 02 | 2783 (+) | 1.000 | 0.918 | agGATAacc | |
| V$CREB 02 | 2937 (+) | 1.000 | 0.901 | catgTGACgtga | |
| V$ATF 01 | 2938 (+) | 1.000 | 0.949 | atgTGACgtgagta | |
| V$CREBP1 Q2 | 2939 (+) | 1.000 | 0.916 | tgTGACgtgagt | |
| V$CREBP1CJUN 01 | 2941 (+) | 1.000 | 0.973 | tgACGTga | |
| V$CREB 01 | 2941 (+) | 1.000 | 0.957 | TGACgtga | |
| V$LMO2COM 01 | 2962 (+) | 1.000 | 0.971 | accCAGGtgccc | |
| V$IK2 01 | 3088 (+) | 1.000 | 0.941 | ggctGGGAgttc | |
| V$CREL 01 | 3091 (+) | 1.000 | 0.945 | tgggagTTCC | |
| V$AP1FJ Q2 | 3109 (+) | 1.000 | 0.932 | aaTGACtccac | |
| V$FREAC7 01 | 3190 (+) | 1.000 | 0.912 | aaaaaaTAAAaaggaa | |
| V$NFAT Q6 | 3198 (+) | 1.000 | 0.954 | aaaagGAAAgaa | |
| V$GATA1 03 | 3252 (+) | 1.000 | 0.968 | ttgtaGATAaaggg | |
| V$GATA2 02 | 3254 (+) | 1.000 | 0.941 | gtaGATAaag | |
| V$GATA3 02 | 3254 (+) | 1.000 | 0.905 | gtaGATAaag | |
| V$LMO2COM 02 | 3255 (+) | 1.000 | 0.959 | taGATAaag | |
| V$MZF1 01 | 3261 (+) | 1.000 | 0.969 | aagGGGGa | |
| V$IK2 01 | 3289 (+) | 1.000 | 0.946 | gtctGGGAgagc | |
| V$AP1FJ Q2 | 3310 (+) | 1.000 | 0.914 | tgTGACcctga | |
| V$IK2 01 | 3352 (+) | 1.000 | 0.915 | gagaGGGAtcga | |
| V$MZF1 01 | 3372 (+) | 1.000 | 0.986 | agaGGGGa | homologous |
| V$IK2 01 | 3372 (+) | 1.000 | 0.901 | agagGGGAacca | regions↓ |
| V$TH1E47 01 | 3428 (+) | 1.000 | 0.910 | caaaatgtCTGGatta | |
| V$FREAC7 01 | 3440 (+) | 1.000 | 0.921 | attataTAAAaaagag | |
| V$OCT1 06 | 3470 (+) | 1.000 | 0.903 | cactttgatATGTt | |
| V$GATA1 02 | 3471 (+) | 1.000 | 0.914 | actttGATAtgtta | |
| V$LMO2COM 02 | 3474 (+) | 1.000 | 0.913 | ttGATAtgt | |
| V$BRN2 01 | 3476 (+) | 1.000 | 0.901 | gatatgttAAATaggc | |
| V$DELTAEF1 01 | 3488 (+) | 1.000 | 0.951 | aggcACCTcag | |
| V$CMYB 01 | 3547 (+) | 1.000 | 0.947 | cctagagaccGTTGttta | |
| V$AP1FJ Q2 | 3569 (+) | 1.000 | 0.902 | gaTGACctctg | |
| V$OCT1 06 | 3597 (+) | 1.000 | 0.903 | cagtcttgcATGTa | |
| V$MZF1 01 | 3715 (+) | 1.000 | 0.956 | ctgGGGGa | |
| V$S8 01 | 3723 (+) | 1.000 | 0.934 | ggcgagaaATTAgcac | |
| V$IK2 01 | 3769 (+) | 1.000 | 0.911 | ctaaGGGAcccc | |
| V$IK2 01 | 3850 (+) | 1.000 | 0.904 | cacaGGGActca | |
| V$LMO2COM 01 | 3887 (+) | 1.000 | 0.949 | agaCAGGtggct | |
| V$MYOD 01 | 3887 (+) | 1.000 | 0.945 | agaCAGGtggct | |
| V$NFAT Q6 | 3913 (+) | 1.000 | 0.959 | ctttgGAAAcat | |
| V$NFAT Q6 | 4008 (+) | 1.000 | 0.933 | gtttgGAAAgtc | |
| V$NKX25 01 | 4063 (+) | 1.000 | 0.917 | ctAAGTg | |
| V$NF1 Q6 | 4101 (+) | 1.000 | 0.915 | catTGGCtgtggtttctg | |
| V$PADS C | 4108 (+) | 1.000 | 0.950 | tGTGGTttc | |
| V$IK1 01 | 4133 (+) | 1.000 | 0.909 | tgatcGGAaaagc | |
| V$IK2 01 | 4133 (+) | 1.000 | 0.947 | tgatGGGAaaag | |
| V$NFAT Q6 | 4134 (+) | 1.000 | 0.944 | gatggGAAAagc | |
| V$IK2 01 | 4145 (+) | 1.000 | 0.954 | ctttGGGAtcct | |
| V$IK1 01 | 4155 (+) | 1.000 | 0.920 | ctctGGGAatcgg | |
| V$IK2 01 | 4155 (+) | 1.000 | 0.961 | ctctGGGAatcg | |
| V$AP4 Q5 | 4179 (+) | 1.000 | 0.916 | aaCAGCagct | |
| V$AP4 Q5 | 4180 (+) | 1.000 | 0.971 | agcAGCtgct | |
| V$HNF3B 01 | 4199 (+) | 1.000 | 0.936 | gcaaaTGTTtccttg | |
| V$HFH2 01 | 4201 (+) | 1.000 | 0.922 | aaaTGTTtcctt | |
| V$MZF1 01 | 4252 (+) | 1.000 | 0.948 | ccaGGGGa | |
| V$AP4 Q5 | 4306 (+) | 1.000 | 0.914 | caCAGCagcc | |
| V$AP4 Q5 | 4332 (+) | 1.000 | 0.909 | aaCAGCtcca | |
| V$DELTAEF1 01 | 4368 (+) | 1.000 | 0.950 | ctgcACCTagc | |
| V$AP4 Q5 | 4416 (+) | 1.000 | 0.935 | tcCAGCtgtg | |
| V$IK2 01 | 4470 (+) | 1.000 | 0.921 | aactGGGAggta | |
| V$MZF1 01 | 4492 (+) | 1.000 | 0.951 | ctcGGGGa | |
| V$IK2 01 | 4492 (+) | 1.000 | 0.919 | ctcgGGGAtttc | |
| V$IK2 01 | 4501 (+) | 1.000 | 0.912 | ttctGGGAggct | homologous |
| V$NF1 Q6 | 4536 (+) | 1.000 | 0.913 | actTGGCtgtctgtaggc | regions ↑ |

The sequence of the mouse (AL663116, position 31673-36359) was analysed in respect of possible DNA binding sites for transcription factors with the aid of the MatInspector computer program (sense Strand, Core Simil.: 1.000, Matrix Simil.: 0.900). The transcription factors which were identified at the same position as in the sequence of the rat are provided in boldface in the table.

TABLE 6

Transcription factor binding sites in the 5′ region upstream of the VR1
exon 1d of humans.

| | | | | | |
|---|---|---|---|---|---|
| Matrix | Position(str) | Core | Matrix | | +HL,49 |
| Name | of Matrix | Simil. | Simil. | Sequence | |

| | | | | | |
|---|---|---|---|---|---|
| V$IK2 01 | 33 (+) | 1.000 | 0.928 | acttGGGAggca | |
| V$GFI1 01 | 145 (+) | 1.000 | 0.935 | aaaaaaaaAATCaaatttaatact | |
| V$SRY 02 | 188 (+) | 1.000 | 0.915 | tctaACAAaacc | |
| V$LMO2COM 02 | 217 (+) | 1.000 | 0.907 | ttGATAttc | |
| V$ARNT 01 | 220 (+) | 1.000 | 0.953 | atattcaCGTGctaaa | |
| V$USF 01 | 221 (+) | 1.000 | 0.982 | tattCACGtgctaa | |
| V$MAX 01 | 221 (+) | 1.000 | 0.941 | tattCACGtgctaa | |
| V$NMYC 01 | 222 (+) | 1.000 | 0.960 | attcaCGTGcta | |
| V$MYCMAX 02 | 222 (+) | 1.000 | 0.917 | attCACGtgcta | |
| V$USF Q6 | 223 (+) | 1.000 | 0.922 | ttCACGtgct | |
| V$USF C | 224 (+) | 1.000 | 0.997 | tCACGTgc | |
| V$NFAT Q6 | 242 (+) | 1.000 | 0.965 | gttagGAAAata | |
| V$GFI1 01 | 338 (+) | 1.000 | 0.948 | aacatacaAATCtgagccacggtg | |
| V$NF1 Q6 | 376 (+) | 1.000 | 0.910 | catTGGCattgcgtgtca | |
| V$AHRARNT 01 | 378 (+) | 1.000 | 0.949 | ttggcattgCGTGtca | |
| V$TCF11 01 | 390 (+) | 1.000 | 0.967 | GTCAtggacatgc | |
| V$CEBPB 01 | 452 (+) | 1.000 | 0.994 | aagttgtGCAAtgt | |
| V$AP1FJ Q2 | 463 (+) | 1.000 | 0.907 | tgTGACatctg | |
| V$DELTAEF1 01 | 491 (+) | 1.000 | 0.974 | aagcACCTtaa | |
| V$GATA1 02 | 512 (+) | 1.000 | 0.922 | cacttGATAttgaa | |
| V$LMO2COM 02 | 515 (+) | 1.000 | 0.937 | ttGATAttg | |
| V$S8 01 | 517 (+) | 1.000 | 0.946 | gatattgaATTAagtt | |
| V$HNF3B 01 | 676 (+) | 1.000 | 0.923 | tttttTGTTtgtttg | |
| V$HFN8 01 | 678 (+) | 1.000 | 0.921 | tttTGTTtgtttg | |
| V$HFH2 01 | 678 (+) | 1.000 | 0.953 | tttTGTTtgttt | |
| V$HFH3 01 | 678 (+) | 1.000 | 0.968 | tttTGTTtgtttg | |
| V$HNF3B 01 | 680 (+) | 1.000 | 0.930 | ttgttTGTTtgtttt | |
| V$HFH2 01 | 682 (+) | 1.000 | 0.963 | gttTGTTtgttt | |
| V$HFH3 01 | 682 (+) | 1.000 | 0.977 | gttTGTTtgtttt | |
| V$HFN8 01 | 682 (+) | 1.000 | 0.908 | gttTGTTtgtttt | |
| V$HFH3 01 | 686 (+) | 1.000 | 0.904 | gttTGTTtttgtt | |
| V$TH1E47 01 | 692 (+) | 1.000 | 0.919 | ttttgtttCTGGctgt | homologous |
| V$LMO2COM 01 | 735 (+) | 1.000 | 0.969 | agcCAGGtgtgg | region ↓ |
| V$TCF11 01 | 759 (+) | 1.000 | 0.973 | GTCAtcccagcac | |
| V$AP1FJ Q2 | 773 (+) | 1.000 | 0.934 | tcTGACacaga | |
| V$CEBPB 01 | 792 (+) | 1.000 | 0.911 | ggttgatGCAAgtc | |
| V$RORA1 01 | 831 (+) | 1.000 | 0.947 | tgttccaGGTCag | |
| V$SRY 02 | 880 (+) | 1.000 | 0.933 | acaaACAAaaat | |
| V$GPI1 01 | 881 (+) | 1.000 | 0.909 | caaacaaaAATCctagagaaactc | |
| V$TCF11 01 | 954 (+) | 1.000 | 0.993 | GTCAttgtggccc | |
| V$NFAT Q6 | 980 (+) | 1.000 | 0.974 | catagGAAAcag | |
| V$AP1FJ Q2 | 1000 (+) | 1.000 | 0.910 | ggTGACcatag | |
| V$STAF 02 | 1070 (+) | 1.000 | 0.935 | ctttCCCAtcatccagagcct | |
| V$IK1 01 | 1088 (+) | 1.000 | 0.911 | cctaGGGAacact | |
| V$IK2 01 | 1088 (+) | 1.000 | 0.949 | cctaGGGAacac | |
| V$AP1FJ Q2 | 1129 (+) | 1.000 | 0.903 | ctTGACttcca | |
| V$DELTAEF1 01 | 1151 (+) | 1.000 | 0.942 | gaacACCTtgt | |
| V$DELTAEF1 01 | 1248 (+) | 1.000 | 0.949 | tgccACCTgtg | |
| V$MYOD Q6 | 1249 (+) | 1.000 | 0.924 | gcCACCtgtg | |
| V$GATA1 04 | 1267 (+) | 1.000 | 0.905 | aaatGATAttcag | |
| V$LMO2COM 02 | 1269 (+) | 1.000 | 0.907 | atGATAttc | |
| V$DELTAEF1 01 | 1303 (+) | 1.000 | 0.952 | ccacACCTgct | |
| V$MYOD Q6 | 1304 (+) | 1.000 | 0.950 | caCACCtgct | |
| V$NF1 Q6 | 1378 (+) | 1.000 | 0.907 | attTGGCatctcagaagc | |
| V$SRY 02 | 1403 (+) | 1.000 | 0.916 | aaaaACAAgaag | |
| V$RFX1 02 | 1447 (+) | 1.000 | 0.906 | aggacaccagtGCAAcca | |
| V$TCF11 01 | 1482 (+) | 1.000 | 0.902 | GTCAcgttgcctg | |
| V$TCF11 01 | 1531 (+) | 1.000 | 0.955 | GTCAtatccagtg | |
| V$DELTAEF1 01 | 1658 (+) | 1.000 | 0.978 | tcacACCTgat | |
| V$MYOD Q6 | 1659 (+) | 1.000 | 0.944 | caCACCtgat | homologous |
| V$TH1E47 01 | 1675 (+) | 1.000 | 0.939 | cactgggtCTGGcaaa | region ↑ |
| V$DELTAEF1 01 | 1752 (+) | 1.000 | 0.958 | ccacACCTcat | |
| V$NKX25 01 | 1773 (+) | 1.000 | 0.938 | tgAAGTg | |
| V$NFY 01 | 1784 (+) | 1.000 | 0.910 | tgagcCCAAtgggata | |
| V$IK2 01 | 1790 (+) | 1.000 | 0.940 | caatGGGAtagt | |
| V$GATA1 02 | 1791 (+) | 1.000 | 0.915 | aatggGATAgtatg | |
| V$NKX25 01 | 1807 (+) | 1.000 | 0.938 | tgAAGTg | |
| V$CMYB 01 | 1862 (+) | 1.000 | 0.902 | cactgctgctGTTGacat | |
| V$IK2 01 | 1883 (+) | 1.000 | 0.949 | aactGGGAccac | |
| V$DELTAEF1 01 | 1897 (+) | 1.000 | 0.939 | agccACCTacc | |
| V$NRF2 01 | 1908 (+) | 1.000 | 0.916 | acaGGAAgtg | |
| V$IK2 01 | 1973 (+) | 1.000 | 0.920 | gacaGGGAaggg | |
| V$LMO2COM 02 | 2015 (+) | 1.000 | 0.911 | gtGATAcct | |
| V$NKX25 01 | 2056 (+) | 1.000 | 0.930 | gtAAGTg | |
| V$E47 01 | 2075 (+) | 1.000 | 0.944 | ggtGCAGgtggcttc | |
| V$MYOD 01 | 2076 (+) | 1.000 | 0.908 | gtgCAGGtggct | |
| V$LMO2COM 01 | 2076 (+) | 1.000 | 0.956 | gtgCAGGtggct | |
| V$NFAT Q6 | 2136 (+) | 1.000 | 0.979 | tagagGAAAagc | |
| V$GATA1 02 | 2159 (+) | 1.000 | 0.937 | gtgatGATAgaaga | |
| V$NFAT Q6 | 2181 (+) | 1.000 | 0.962 | agtagGAAAaga | |
| V$AP4 Q5 | 2238 (+) | 1.000 | 0.907 | ttCAGCagtg | |
| V$MZF1 01 | 2263 (+) | 1.000 | 0.956 | ctgGGGGa | |
| V$IK2 01 | 2263 (+) | 1.000 | 0.912 | ctggGGGAagga | |
| V$AP1 Q2 | 2279 (+) | 1.000 | 0.902 | gaTGACtggtg | |
| V$IK2 01 | 2297 (+) | 1.000 | 0.920 | agtaGGGAtgga | |
| V$AP4 Q5 | 2325 (+) | 1.000 | 0.956 | ccCAGCtgag | |
| V$IK2 01 | 2336 (+) | 1.000 | 0.901 | gagaGGGActgg | |
| V$IK2 01 | 2360 (+) | 1.000 | 0.900 | aggaGGGAtggg | |
| V$IK2 01 | 2369 (+) | 1.000 | 0.934 | gggtGGGAggcc | |
| V$CREB 02 | 2414 (+) | 1.000 | 0.937 | aggaTGACgaca | |
| V$AP1FJ Q2 | 2416 (+) | 1.000 | 0.923 | gaTGACgacaa | |
| V$GATA3 03 | 2426 (+) | 1.000 | 0.923 | agAGATcgta | |
| V$MZF1 01 | 2475 (+) | 1.000 | 0.948 | tcaGGGGa | |
| V$TCF11 01 | 2489 (+) | 1.000 | 0.982 | GTCAtggatgctt | |
| V$NKX25 02 | 2499 (+) | 1.000 | 0.971 | ctTAATtc | |
| V$AP4 Q5 | 2531 (+) | 1.000 | 0.947 | acCAGCtgag | |
| V$GATA1 02 | 2593 (+) | 1.000 | 0.921 | agagaGATAcaagg | |
| V$GATA2 03 | 2595 (+) | 1.000 | 0.931 | agaGATAcaa | |
| V$LMO2COM 02 | 2596 (+) | 1.000 | 0.913 | gaGATAcaa | |
| V$IK2 01 | 2637 (+) | 1.000 | 0.933 | gtttGGGAggtg | |
| V$LYF1 01 | 2638 (+) | 1.000 | 0.989 | tttGGGAgg | |
| V$GATA1 02 | 2665 (+) | 1.000 | 0.932 | ttggtGATAgcaga | |
| V$LMO2COM 02 | 2668 (+) | 1.000 | 0.921 | gtGATAgca | |
| V$NKX25 01 | 2722 (+) | 1.000 | 0.938 | tgAAGTg | |
| V$GATA1 02 | 2729 (+) | 1.000 | 0.932 | tttagGATAatgaa | |
| V$LMO2COM 02 | 2732 (+) | 1.000 | 0.932 | agGATAatg | |
| V$SRY 02 | 2776 (+) | 1.000 | 0.902 | aaaaACAAgaac | |
| V$TCF11 01 | 2874 (+) | 1.000 | 0.973 | GTCAtgtgatctg | |
| V$TCF11 01 | 2890 (+) | 1.000 | 0.973 | GTCAtgtgatctg | |
| V$DELTAEF1 01 | 2913 (+) | 1.000 | 0.934 | taacACCTccg | |
| V$IK2 01 | 3042 (+) | 1.000 | 0.927 | ggctGGGAgtta | |
| V$AP1FJ Q2 | 3063 (+) | 1.000 | 0.939 | gaTGACtccac | |
| V$CP2 01 | 3125 (+) | 1.000 | 0.951 | gctccatCCAG | |
| V$FREAC7 01 | 3161 (+) | 1.000 | 0.912 | aagaaaTAAAaaggaa | |
| V$NFAT Q6 | 3169 (+) | 1.000 | 0.954 | aaaagGAAAgaa | |
| V$GATA1 03 | 3223 (+) | 1.000 | 0.968 | ttgtaGATAaaggg | |
| V$GATA2 02 | 3225 (+) | 1.000 | 0.941 | gtaGATAaag | |
| V$GATA3 02 | 3225 (+) | 1.000 | 0.905 | gtaGATAaag | |
| V$LMO2COM 02 | 3226 (+) | 1.000 | 0.959 | taGATAaag | |
| V$IK2 01 | 3260 (+) | 1.000 | 0.924 | gtctGGGAgagt | |
| V$MZF1 01 | 3297 (+) | 1.000 | 0.989 | tgtGGGGa | |
| V$MZF1 01 | 3344 (+) | 1.000 | 0.986 | agaGGGGa | homologous |
| V$IK2 01 | 3344 (+) | 1.000 | 0.901 | agagGGGAacca | region ↓ |
| V$IK2 01 | 3390 (+) | 1.000 | 0.935 | ggtaGGGAacca | |
| V$GATA3 03 | 3408 (+) | 1.000 | 0.942 | atAGATtata | |
| V$IK2 01 | 3416 (+) | 1.000 | 0.929 | tataGGGAagag | homologous |
| V$TH1E47 01 | 3423 (+) | 1.000 | 0.905 | aagagcctCTGGcaga | region ↑ |
| V$GATA1 02 | 3467 (+) | 1.000 | 0.947 | ttcagGATAgaggg | |
| V$GATA1 03 | 3467 (+) | 1.000 | 0.917 | ttcagGATAgaggg | |
| V$GATA1 04 | 3468 (+) | 1.000 | 0.909 | tcagGATAgaggg | |
| V$LMO2COM 02 | 3470 (+) | 1.000 | 0.926 | agGATAgag | |
| V$IK2 01 | 3509 (+) | 1.000 | 0.930 | ggtaGGGAttga | |
| V$IK2 01 | 3525 (+) | 1.000 | 0.935 | tgctGGGAgaac | |
| V$RORA1 01 | 3535 (+) | 1.000 | 0.934 | acctagaGGTCag | |
| V$OCT1 06 | 3551 (+) | 1.000 | 0.939 | cactttgacATGTt | homologous |
| V$BRN2 01 | 3557 (+) | 1.000 | 0.958 | gacatgttAAATaggc | regions↓ |
| V$SRY 02 | 3627 (+) | 1.000 | 0.903 | taatACAAttca | |
| V$CMYB 01 | 3653 (+) | 1.000 | 0.972 | cctagtggcaGTTGcttg | |
| V$BARBIE 01 | 3720 (+) | 1.000 | 0.915 | cctgAAAGctggtgg | |
| V$CREL 01 | 3732 (+) | 1.000 | 0.968 | tggactTTCC | |
| V$NFKAPPAB65 01 | 3732 (+) | 1.000 | 0.967 | tggactTTCC | |
| V$IK1 01 | 3750 (+) | 1.000 | 0.903 | atctGGGAaggag | |
| V$IK2 01 | 3750 (+) | 1.000 | 0.959 | atctGGGAagga | |
| V$S8 01 | 3843 (+) | 1.000 | 0.934 | ggtgagaaATTAgcac | |
| V$MYOD 01 | 4017 (+) | 1.000 | 0.945 | agaCAGGtggct | |
| V$LMO2COM 01 | 4017 (+) | 1.000 | 0.949 | agaCAGGtggct | |
| V$TCF11 01 | 4040 (+) | 1.000 | 0.964 | GTCAtttcccttt | |
| V$NFAT Q6 | 4144 (+) | 1.000 | 0.963 | gtttgGAAAatc | |
| V$GFI1 01 | 4144 (+) | 1.000 | 0.944 | gtttggaaAATCaaggctccaaga | |
| V$NKX25 01 | 4206 (+) | 1.000 | 0.917 | ctAAGTg | |
| V$DELTAEF1 01 | 4210 (+) | 1.000 | 0.940 | gtgcACCTcgg | |
| V$NF1 Q6 | 4244 (+) | 1.000 | 0.915 | catTGGCtgtggtttctg | |
| V$PADS C | 4251 (+) | 1.000 | 0.950 | tGTGGTttc | |
| V$IK1 01 | 4276 (+) | 1.000 | 0.909 | tgatGGGAaaagc | |
| V$IK2 01 | 4276 (+) | 1.000 | 0.947 | tgatGGGAaaag | |
| V$NFAT Q6 | 4277 (+) | 1.000 | 0.944 | gatggGAAAagc | |
| V$IK2 01 | 4288 (+) | 1.000 | 0.954 | ctttGGGAtcct | |
| V$IK2 01 | 4298 (+) | 1.000 | 0.961 | ctctGGGAatcg | |
| V$IK1 01 | 4298 (+) | 1.000 | 0.920 | ctctGGGAatcgg | |
| V$RFX1 02 | 4307 (+) | 1.000 | 0.962 | tcggagccgtgGCAAcag | |
| V$RFX1 01 | 4308 (+) | 1.000 | 0.905 | cggagccgtgGCAAcag | |
| V$AP4 Q5 | 4320 (+) | 1.000 | 0.916 | agCAGCtgct | |
| V$AP4 Q5 | 4323 (+) | 1.000 | 0.971 | aaCAGCagct | |
| V$HNF3B 01 | 4342 (+) | 1.000 | 0.936 | gcaaaTGTTtccttg | |
| V$HFH2 01 | 4344 (+) | 1.000 | 0.922 | aaaTGTTtcctt | |
| V$MZF1 01 | 4395 (+) | 1.000 | 0.948 | ccaGGGGa | |
| V$AP4 Q5 | 4445 (+) | 1.000 | 0.914 | caCAGCagcc | |
| V$AP4 Q5 | 4471 (+) | 1.000 | 0.909 | aaCAGCtcca | |
| V$DELTAEF1 01 | 4507 (+) | 1.000 | 0.950 | ctgcACCTagc | |
| V$AP4 Q5 | 4555 (+) | 1.000 | 0.935 | tcCAGCtgtg | |
| V$IK2 01 | 4639 (+) | 1.000 | 0.912 | ttctGGGAggct | |
| V$RFX1 02 | 4643 (+) | 1.000 | 0.915 | gggaggctgaaGCAAcag | homologous |
| V$CEBPB 01 | 4647 (+) | 1.000 | 0.903 | ggctgaaGCAAcag | regions ↑ |

The sequence of humans (AF168787, position 36616-33151) was analysed in respect of possible DNA binding sites for transcription factors with the aid of the MatInspector computer program (sense Strand, Core Simil.: 1.000, Matrix Simil.: 0.900).

| | | | | |
|---|---|---|---|---|
| V$IK2 01 | 13 (+) | 1.000 | 0.911 | ttgtGGGAggtt |
| V$IK2 01 | 27 (+) | 1.000 | 0.914 | accaGGGAaagg |
| V$NFAT Q6 | 28 (+) | 1.000 | 0.909 | ccaggGAAAgga |
| V$TCF11 01 | 50 (+) | 1.000 | 0.990 | GTCAttcaggtga |
| V$LMO2COM 01 | 53 (+) | 1.000 | 0.919 | attCAGGtgaga |
| V$IK2 01 | 84 (+) | 1.000 | 0.906 | aggaGGGAtgga |
| V$RORA1 01 | 137 (+) | 1.000 | 0.930 | ggctggaGGTCac |
| V$IK2 01 | 196 (+) | 1.000 | 0.916 | ggcaGGGActgc |
| V$MZF1 01 | 219 (+) | 1.000 | 0.982 | ggaGGGGa |
| V$IK2 01 | 234 (+) | 1.000 | 0.912 | tggaGGGAacag |
| V$MZF1 01 | 266 (+) | 1.000 | 0.980 | cggGGGGa |
| V$DELTAEF1 01 | 287 (+) | 1.000 | 0.971 | cttcACCTgca |
| V$MYOD Q6 | 288 (+) | 1.000 | 0.908 | ttCACCtgca |
| V$IK2 01 | 308 (+) | 1.000 | 0.909 | ggcaGGGAtgga |
| V$IK2 01 | 321 (+) | 1.000 | 0.923 | ctcaGGGAagag |
| V$AP4 Q5 | 354 (+) | 1.000 | 0.902 | agCAGCcgct |
| V$AP1 Q2 | 361 (+) | 1.000 | 0.948 | gcTGACttgga |
| V$IK2 01 | 386 (+) | 1.000 | 0.924 | ccttGGGAgggg |
| V$EVI1 02 | 432 (+) | 1.000 | 0.949 | ggagAAGAtaa |
| V$GATA1 04 | 434 (+) | 1.000 | 0.978 | agaaGATAaggct |
| V$GATA3 02 | 435 (+) | 1.000 | 0.924 | gaaGATAagg |
| V$GATA2 02 | 435 (+) | 1.000 | 0.969 | gaaGATAagg |
| V$LMO2COM 02 | 436 (+) | 1.000 | 0.990 | aaGATAagg |
| V$E47 02 | 464 (+) | 1.000 | 0.905 | accccCAGGtgtgggg |
| V$LMO2COM 01 | 466 (+) | 1.000 | 0.985 | cccCAGGtgtgg |
| V$MZF1 01 | 473 (+) | 1.000 | 0.989 | tgtGGGGa |
| V$IK2 01 | 473 (+) | 1.000 | 0.908 | tgtgGGGAacgg |
| V$VMYB 02 | 477 (+) | 1.000 | 0.928 | gggAACGgc |
| V$IK2 01 | 501 (+) | 1.000 | 0.905 | gccaGGGAgtcc |
| V$IK2 01 | 540 (+) | 1.000 | 0.924 | ggccGGGActtc |
| V$NFKB Q6 | 542 (+) | 1.000 | 0.917 | ccGGGActtcccct |
| V$CREL 01 | 543 (+) | 1.000 | 0.949 | cgggacTTCC |
| V$NFKB C | 543 (+) | 1.000 | 0.948 | cGGGACttcccc |
| V$NFKAPPAB 01 | 544 (+) | 1.000 | 0.985 | GGGActtccc |
| V$GATA1 02 | 591 (+) | 1.000 | 0.933 | ccggtGATActgtt |
| V$LM02COM 02 | 594 (+) | 1.000 | 0.943 | gtGATActg |
| V$XFD3 01 | 617 (+) | 1.000 | 0.905 | tctgttAACAaaga |
| V$SRY 02 | 620 (+) | 1.000 | 0.925 | gttaACAAagag |
| V$NFAT Q6 | 664 (+) | 1.000 | 0.954 | cactgGAAAtgg |
| V$HNF3B 01 | 696 (+) | 1.000 | 0.923 | cgtttTGTTtttttt |
| V$HFH2 01 | 698 (+) | 1.000 | 0.938 | tttTGTTttttt |
| V$HFH3 01 | 698 (+) | 1.000 | 0.924 | tttTGTTtttttt |
| V$AP4 Q5 | 758 (+) | 1.000 | 0.926 | ctCAGCtcac |
| V$NKX25 01 | 789 (+) | 1.000 | 1.000 | tcAAGTg |
| V$IK2 01 | 821 (+) | 1.000 | 0.950 | agctGGGActac |
| V$AHRARNT 01 | 827 (+) | 1.000 | 0.905 | gactacaggCGTGcac |
| V$NF1 Q6 | 894 (+) | 1.000 | 0.916 | tgtTGGCtaggctggtct |
| V$GFI1 01 | 1010 (+) | 1.000 | 0.932 | gaggcagaAATCactttggaggct |
| V$CEBPB 01 | 1030 (+) | 1.000 | 0.906 | ggctaagGCAAtgg |
| V$NFAT Q6 | 1102 (+) | 1.000 | 0.933 | agaagGAAAtga |
| V$RORA1 01 | 1131 (+) | 1.000 | 0.911 | gttgagaGGTCac |
| V$NFE2 01 | 1147 (+) | 1.000 | 0.902 | tgCTGAgtctt |
| V$NFAT Q6 | 1202 (+) | 1.000 | 0.940 | gaatgGAAAgca |
| V$GATA1 04 | 1222 (+) | 1.000 | 0.934 | accaGATAtttgc |
| V$LMO2COM 02 | 1224 (+) | 1.000 | 0.917 | caGATAttt |
| V$TCF11 01 | 1259 (+) | 1.000 | 0.902 | GTCActatggcca |
| V$NKX25 01 | 1286 (+) | 1.000 | 0.932 | ccAAGTg |
| V$AP4 Q5 | 1296 (+) | 1.000 | 0.991 | atCAGCtggt |
| V$GKLF 01 | 1368 (+) | 1.000 | 0.922 | aaaaggaaggAGGG |
| V$IK2 01 | 1416 (+) | 1.000 | 0.931 | ctttGGGAggct |
| V$E47 01 | 1428 (+) | 1.000 | 0.903 | gagGCAGgtgaatca |
| V$LMO2COM 01 | 1429 (+) | 1.000 | 0.941 | aggCAGGtgaat |
| V$RORA1 01 | 1439 (+) | 1.000 | 0.942 | atcacaaGGTCag |
| V$S8 01 | 1494 (+) | 1.000 | 0.941 | tactaaaaATTAgttg |
| V$MZF1 01 | 1625 (+) | 1.000 | 0.956 | ctgGGGGa |
| V$NFAT Q6 | 1675 (+) | 1.000 | 0.959 | aaaagGAAAttc |
| V$NFAT Q6 | 1714 (+) | 1.000 | 0.948 | ttgagGAAAtta |
| V$S8 01 | 1714 (+) | 1.000 | 0.952 | ttgaggaaATTAtgct |
| V$NKX25 01 | 1728 (+) | 1.000 | 0.917 | ctAAGTg |
| V$IK2 01 | 1853 (+) | 1.000 | 0.939 | ggtcGGGAaggg |
| V$MZF1 01 | 1859 (+) | 1.000 | 0.960 | gaaGGGGa |
| V$IK2 01 | 1895 (+) | 1.000 | 0.959 | gtttGGAtgat |
| V$BRN2 01 | 1986 (+) | 1.000 | 0.956 | aacatggtTAATacgg |
| V$FREAC7 01 | 2039 (+) | 1.000 | 0.955 | aatataTAAAtatata |
| V$XFD2 01 | 2041 (+) | 1.000 | 0.922 | tataTAAAtatata |
| V$XFD1 01 | 2041 (+) | 1.000 | 0.904 | tataTAAAtatata |
| V$TATA 01 | 2042 (+) | 1.000 | 0.923 | ataTAAAtatatatt |
| V$DELTAEF1 01 | 2124 (+) | 1.000 | 0.939 | ctccACCTccc |
| V$NKX25 01 | 2139 (+) | 1.000 | 1.000 | tcAAGTg |
| V$IK2 01 | 2171 (+) | 1.000 | 0.950 | agctGGGActac |
| V$E47 02 | 2177 (+) | 1.000 | 0.906 | gactaCAGGtgcccac |
| V$LMO2COM 01 | 2179 (+) | 1.000 | 0.976 | ctaCAGGtgccc |
| V$MYOD 01 | 2179 (+) | 1.000 | 0.910 | ctaCAGGtgccc |
| V$E47 02 | 2270 (+) | 1.000 | 0.910 | gaccctCAGGtgatcca |
| V$MYOD 01 | 2272 (+) | 1.000 | 0.903 | cctCAGGtgatc |
| V$LMO2COM 01 | 2272 (+) | 1.000 | 0.951 | cctCAGGtgatc |
| V$DELTAEF1 01 | 2281 (+) | 1.000 | 0.959 | atccACCTgcc |
| V$MYOD Q6 | 2282 (+) | 1.000 | 0.992 | tcCACCtgcc |
| V$FREAC7 01 | 2351 (+) | 1.000 | 0.964 | aattataTAAAcaagaa |
| V$XFD2 01 | 2353 (+) | 1.000 | 0.975 | tttaTAAAcaagaa |
| V$TATA 01 | 2354 (+) | 1.000 | 0.908 | ttaTAAAcaagaatg |
| V$SRY 02 | 2356 (+) | 1.000 | 0.912 | ataaACAAgaat |
| V$NKX25 02 | 2384 (+) | 1.000 | 0.903 | atTAATtg |
| V$AP1FJ Q2 | 2399 (+) | 1.000 | 0.919 | caTGACacaca |
| V$FREAC7 01 | 2409 (+) | 1.000 | 0.945 | atagcaTAAAcaggtg |
| V$XFD2 01 | 2411 (+) | 1.000 | 0.908 | agcaTAAAcaggtg |
| V$E47 02 | 2414 (+) | 1.000 | 0.933 | ataaaCAGGtgtctaa |
| V$MYOD 01 | 2416 (+) | 1.000 | 0.945 | aaaCAGGtgtct |
| V$LMO2COM 01 | 2416 (+) | 1.000 | 0.944 | aaaCAGGtgtct |
| V$IK2 01 | 2468 (+) | 1.000 | 0.952 | ctttGGGAggcc |
| V$E47 01 | 2480 (+) | 1.000 | 0.934 | gagGCAGgtggatca |
| V$LMO2COM 01 | 2481 (+) | 1.000 | 0.957 | aggCAGGtggat |
| V$SREBP1 01 | 2490 (+) | 1.000 | 0.932 | gaTCACttgag |
| V$RORA1 01 | 2493 (+) | 1.000 | 0.928 | cacttgaGGTCag |
| V$T3R 01 | 2494 (+) | 1.000 | 0.912 | acttgaGGTCaggagt |
| V$AP1FJ Q2 | 2521 (+) | 1.000 | 0.918 | ccTGACcaaca |
| V$S8 01 | 2557 (+) | 1.000 | 0.951 | acacaaaaATTAgcca |
| V$ARNT 01 | 2578 (+) | 1.000 | 0.978 | ggtggcaCGTGcctgt |
| V$MAX 01 | 2579 (+) | 1.000 | 0.935 | gtggCACGtgcctg |
| V$USF 01 | 2579 (+) | 1.000 | 0.985 | gtggCACGtgcctg |
| V$MYCMAX 02 | 2580 (+) | 1.000 | 0.914 | tggCACGtgcct |
| V$NMYC 01 | 2580 (+) | 1.000 | 0.971 | tggcaCGTGcct |
| V$USF C | 2582 (+) | 1.000 | 0.994 | gCACGTgc |
| V$IK2 01 | 2604 (+) | 1.000 | 0.921 | acttGGGAggct |
| V$IK2 01 | 2636 (+) | 1.000 | 0.915 | acctGGGAggca |
| V$IK2 01 | 2686 (+) | 1.000 | 0.922 | gcctGGGAgaca |
| V$AP1 Q4 | 2734 (+) | 1.000 | 0.989 | agTGACtaagc |
| V$AHRARNT 01 | 2757 (+) | 1.000 | 0.905 | ggggtgtggCGTGgtg |
| V$IK2 01 | 2768 (+) | 1.000 | 0.930 | tggtGGGAtggg |
| V$S8 01 | 2810 (+) | 1.000 | 0.972 | cctgggcaATTAtcta |
| V$S8 01 | 2818 (+) | 1.000 | 0.986 | attatctaATTAtcgg |
| V$CMYB 01 | 2823 (+) | 1.000 | 0.901 | ctaattatcgGTTGtcta |
| V$DELTAEF1 01 | 2861 (+) | 1.000 | 0.977 | tctcACCTgta |
| V$MYOD Q6 | 2862 (+) | 1.000 | 0.909 | ctCACCtgta |
| V$IK1 01 | 2935 (+) | 1.000 | 0.943 | gtttGGGAaagct |
| V$IK2 01 | 2935 (+) | 1.000 | 0.994 | gtttGGGAaagc |
| V$NFAT Q6 | 2936 (+) | 1.000 | 0.917 | tttggGAAAgct |
| V$OCT1 06 | 2983 (+) | 1.000 | 0.906 | cacacttcaATGCc |
| V$AP1 Q4 | 2996 (+) | 1.000 | 0.924 | ctTGACtcagg |
| V$NF1 Q6 | 3095 (+) | 1.000 | 0.923 | tgtTGGCgtcccgcaggc |
| V$AP2 Q6 | 3102 (+) | 1.000 | 0.924 | gtCCCGcaggca |
| V$AP4 Q5 | 3110 (+) | 1.000 | 0.971 | ggCAGCtgct |
| V$IK2 01 | 3193 (+) | 1.000 | 0.932 | gtctGGGAgaga |
| V$AP1FJ Q2 | 3236 (+) | 1.000 | 0.930 | tgTGACtctct |
| V$NKX25 01 | 3298 (+) | 1.000 | 0.938 | tgAAGTg |
| V$GFI1 01 | 3345 (+) | 1.000 | 0.905 | acgcctggAATCccagcactttgg |
| V$IK2 01 | 3363 (+) | 1.000 | 0.952 | ctttGGGAggcc |
| V$E47 01 | 3375 (+) | 1.000 | 0.934 | gagGCAGgtggatga |
| V$LMO2COM 01 | 3376 (+) | 1.000 | 0.957 | aggCAGGtggat |
| V$CREB 02 | 3383 (+) | 1.000 | 0.930 | tggaTGACgagg |
| V$AP1FJ Q2 | 3385 (+) | 1.000 | 0.910 | gaTGACgaggt |
| V$RORA1 01 | 3386 (+) | 1.000 | 0.936 | atgacgaGGTCag |
| V$AP1FJ Q2 | 3433 (+) | 1.000 | 0.905 | ccTGACtctac |
| V$S8 01 | 3449 (+) | 1.000 | 0.977 | atacaacaATTAgctg |
| V$SRY 02 | 3450 (+) | 1.000 | 0.921 | tacaACAAttag |
| V$SOX5 01 | 3451 (+) | 1.000 | 0.980 | acaaCAATta |
| V$E47 01 | 3471 (+) | 1.000 | 0.909 | atgGCAGgtgcctgc |
| V$LMO2COM 01 | 3472 (+) | 1.000 | 0.967 | tggCAGGtgcct |
| V$IK2 01 | 3496 (+) | 1.000 | 0.905 | attcGGGAggct |
| V$IK2 01 | 3528 (+) | 1.000 | 0.908 | acctGGGAggtg |
| V$NFAT Q6 | 3622 (+) | 1.000 | 0.947 | aaaagGAAAtga |
| V$AP1FJ Q2 | 3629 (+) | 1.000 | 0.907 | aaTGACactga |
| V$GATA1 04 | 3634 (+) | 1.000 | 0.936 | cactGATAgttat |
| V$LMO2COM 02 | 3636 (+) | 1.000 | 0.908 | ctGATAgtt |
| V$IK2 01 | 3690 (+) | 1.000 | 0.922 | ggctGGGAcctg |
| V$AP1FJ Q2 | 3702 (+) | 1.000 | 0.956 | gcTGACccaga |
| V$MZF1 01 | 3744 (+) | 1.000 | 0.965 | tttGGGGa |
| V$IK2 01 | 3776 (+) | 1.000 | 0.919 | gttaGGGActag |
| V$VMYB 01 | 3879 (+) | 1.000 | 0.937 | gaaAACGgaa |
| V$CREB 02 | 3905 (+) | 1.000 | 0.907 | gtttTGACgtcg |
| V$ATF 01 | 3906 (+) | 1.000 | 0.947 | tttTGACgtcgctg |
| V$CREBP1 Q2 | 3907 (+) | 1.000 | 0.902 | ttTGACgtcgct |
| V$CREB 01 | 3909 (+) | 1.000 | 0.974 | TGACgtcg |
| V$TCF11 01 | 3929 (+) | 1.000 | 0.977 | GTCAtttgtggag |
| V$AP4 Q5 | 4020 (+) | 1.000 | 0.902 | tgCAGCtctg |
| V$NKX25 01 | 4040 (+) | 1.000 | 0.930 | gtAAGTg |
| V$AP4 Q5 | 4076 (+) | 1.000 | 0.916 | ggCAGCagct |
| V$NKX25 01 | 4084 (+) | 1.000 | 0.917 | ctAAGTg |
| V$PADS C | 4087 (+) | 1.000 | 0.939 | aGTGGTttc |
| V$IK1 01 | 4112 (+) | 1.000 | 0.909 | tgatGGGAaaagc |
| V$IK2 01 | 4112 (+) | 1.000 | 0.947 | tgatGGGAaaag |
| V$NFAT Q6 | 4113 (+) | 1.000 | 0.944 | gatggGAAAagc |
| V$IK2 01 | 4124 (+) | 1.000 | 0.954 | ctttGGGAtcct |
| V$GFI1 01 | 4133 (+) | 1.000 | 0.941 | cctctgggAATCagagccgcagca |
| V$IK1 01 | 4134 (+) | 1.000 | 0.922 | ctctGGGAatcag |
| V$IK2 01 | 4134 (+) | 1.000 | 0.967 | ctctGGGAatca |
| V$AP4 Q5 | 4159 (+) | 1.000 | 0.971 | ggCAGCtgct |
| V$AP4 Q5 | 4235 (+) | 1.000 | 0.971 | ggCAGCtgct |
| V$IK2 01 | 4256 (+) | 1.000 | 0.917 | gcccGGGAcccc |
| V$MZF1 01 | 4273 (+) | 1.000 | 0.982 | ggcGGGGa |
| V$USF Q6 | 4295 (+) | 1.000 | 0.901 | caCACGagcc |
| V$AP4 Q5 | 4303 (+) | 1.000 | 0.905 | ccCAGCtctc |
| V$NFY Q6 | 4403 (+) | 1.000 | 0.904 | tggCCAAtgca |
| V$AP4 Q5 | 4469 (+) | 1.000 | 0.962 | ccCAGCtgtg |
| V$DELTAEF1 01 | 4496 (+) | 1.000 | 0.954 | actcACCTctc |
| V$VMYB 01 | 4528 (+) | 1.000 | 0.901 | gaaAACGggg |
| V$DELTAEF1 01 | 4614 (+) | 1.000 | 0.956 | aagcACCTggg |
| V$MYOD Q6 | 4615 (+) | 1.000 | 0.917 | agCACCtggg |
| V$IK2 01 | 4618 (+) | 1.000 | 0.908 | acctGGGAggtg |
| V$AP4 Q5 | 4675 (+) | 1.000 | 0.906 | atCAGCcgtc |
| V$MZF1 01 | 4686 (+) | 1.000 | 0.953 | tcgGGGGa |
| V$AP4 Q5 | 4700 (+) | 1.000 | 0.953 | tgCAGCtgct |
| V$CETS1P54 01 | 4730 (+) | 1.000 | 0.940 | gcCGGAggtt |
| V$IK2 01 | 4779 (+) | 1.000 | 0.955 | ggctGGGAagca |
| V$IK1 01 | 4842 (+) | 01.000 | 0.929 | ggttGGGAagccc |
| V$IK2 01 | 4842 (+) | 1.000 | 0.982 | ggttGGGAagcc |
| V$IK2 01 | 4878 (+) | 1.000 | 0.913 | agaaGGGActac |
| V$MZF1 01 | 5021 (+) | 1.000 | 0.954 | gcaGGGGa |
| V$MZF1 01 | 5055 (+) | 1.000 | 0.976 | attGGGGa |
| V$TH1E47 01 | 5095 (+) | 1.000 | 0.903 | tatctgttCTGGcttt |
| V$GATA3 03 | 5126 (+) | 1.000 | 0.932 | tcAGATcata |
| V$GFI1 01 | 5251 (+) | 1.000 | 0.960 | tttgcctaAATCacggtagaagtt |
| V$AP1FJ Q2 | 5301 (+) | 1.000 | 0.906 | ggTGACaggtg |
| V$LMO2COM 01 | 5303 (+) | 1.000 | 0.964 | tgaCAGGtgcat |
| V$AP1FJ Q2 | 5367 (+) | 1.000 | 0.902 | ccTGACcctgt |
| V$CP2 01 | 5384 (+) | 1.000 | 0.900 | gcccagcCCAG |
| V$IK2 01 | 5452 (+) | 1.000 | 0.938 | agtaGGGAatca |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations falling within the scope of the appended claims and equivalents thereof.

What is claimed is:

1. A nucleic acid comprising a sequence section which contains at least one region which modulates the expression of the VR1 receptor, said sequence section having a sequence selected from the group consisting of: FIG. 3 (SEQ ID NO: 7); FIG. 4 (SEQ ID NO: 8); GenBank Accession Number AL670399, positions 221931 to 223344; GenBank Accession Number AL663116, positions 31673 to 36359; GenBank Accession Number AF168787, positions 44731 to 43231; and GenBank Accession Number AF168787, positions 36616 to 33151, or a homologous derivative, allele or fragment thereof which modulates the expression of the VR1 receptor, or a sequence which hybridizes with one of the foregoing under standard conditions.

2. A nucleic acid according to claim 1, wherein the region which modulates the expression of the VR1 receptor comprises a transcription factor binding site.

3. A nucleic acid according to claim 2, wherein the sequence section comprises one or more binding motifs for a transcription factor selected from the group consisting of MZF1, NFkappaB, GATA 1/2/3, IK 2, NFAT, AP4, SRY, SOX5, CP2, cMyb, SREBP1, deltaEF1, MyoD, GKLF, NRF2, NF1, CETS1P54, NFY TH1E47, RORA1, GFI1, AP1, GATA 1, TCF11, 4255), IK2/1, Brn2, S8, HNF3B and HFH2.

4. A nucleic acid according to claim 1, wherein said nucleic acid is a double-stranded DNA molecule.

5. A nucleic acid according to claim 1, wherein said nucleic acid contains at least one of modified internucleotide bonds or modified nucleobases.

6. A nucleic acid according to claim 1, wherein said nucleic acid comprises about 13 to about 65 nucleotides or base pairs.

7. A nucleic acid according to claim 1, wherein the sequence section comprises the sequence shown in FIG. 3 (SEQ ID NO: 7) or a derivative, allele or fragment thereof which modulates the expression of the VR1 receptor, or a sequence which hybridizes thereto under standard conditions.

8. A nucleic acid according to claim 1, wherein the sequence section comprises a sequence which hybridizes under stringent conditions to the sequence shown in FIG. 3 (SEQ ID NO: 7) or a derivative, allele or fragment thereof which modulates the expression of the VR1 receptor.

9. A nucleic acid according to claim 1, wherein the sequence section comprises the sequence shown in FIG. 4 (SEQ ID NO: 8) or a derivative, allele or fragment thereof which modulates the expression of the VR1 receptor, or a sequence which hybridizes thereto under standard conditions.

10. A nucleic acid according to claim 1, wherein the sequence section comprises a sequence which hybridizes under stringent conditions to the sequence shown in FIG. 4 (SEQ ID NO: 8) or a derivative, allele or fragment thereof which modulates the expression of the VR1 receptor.

11. A nucleic acid according to claim 1, wherein the sequence section comprises the nucleotides of positions 1 to 1423 of the sequence shown in FIG. 3 (SEQ ID NO: 7) or a derivative, allele or fragment thereof which modulates the expression of the VR1 receptor, or a sequence which hybridizes thereto under standard conditions.

12. A nucleic acid according to claim 1, wherein the sequence section comprises the nucleotides of positions 1 to 4549 of the sequence shown in FIG. 4 (SEQ ID NO: 8) or a derivative, allele or fragment thereof which modulates the expression of the VR1 receptor, or a sequence which hybridizes thereto under standard conditions.

13. A nucleic acid according to claim 1, wherein the sequence section comprises the nucleotides of positions 4060 to 4219 of the sequence shown in FIG. 4 (SEQ ID NO: 8) or a derivative, allele or fragment thereof which modulates the expression of the VR1 receptor, or a sequence which hybridizes thereto under standard conditions.

14. A vector containing a nucleic acid according to claim 1.

15. A host cell which is transformed with the vector according to claim 14.

16. A host cell according to claim 15, wherein the host cell is a human germ cell or a human embryonic stem cell.

17. A host cell according to claim 15, wherein the host cell is not a human germ cell or a human embryonic stem cell.

18. A host cell according to claim 15, wherein the host cell is a mammalian cell.

19. A host cell according to claim 15, wherein the host cell is a human cell.

20. A method for modulating the expression of a VR1 receptor comprising: introducing a nucleic acid according to claim 1 into a cell containing a VR1 gene.

21. A method for modulating the expression of a VR1 receptor comprising: introducing a vector according to claim 14 into a cell containing a VR1 gene.

22. A pharmaceutical formulation comprising a nucleic acid according to claim 1 and a pharmaceutically acceptable carrier or adjuvant.

23. A pharmaceutical formulation comprising a vector according to claim 14 and a pharmaceutically acceptable carrier or adjuvant.

24. A pharmaceutical formulation comprising a host cell according to claim 15 and a pharmaceutically acceptable carrier or adjuvant.

25. A method of alleviating pain in a mammal, said method comprising administering to said mammal an effective pain alleviating amount of a nucleic acid according to claim 1.

26. A method of alleviating pain in a mammal, said method comprising administering to said mammal an effective pain alleviating amount of a vector according to claim 14.

27. A method of treating a sensibility disorder associated with the activity of the VR1 receptor in a mammal, said method comprising administering to said mammal an effective amount of a nucleic acid according to claim 1.

28. The method of claim 27, wherein the sensibility disorder is an analgesia, hypalgesia or hyperalgesia.

29. A method of treating sensibility disorders associated with the activity of the VR1 receptor in a mammal, said method comprising administering to said mammal an effective amount of vector according to claim 14.

30. The method of claim 29, wherein the sensibility disorder is an analgesia, hypalgesia or hyperalgesia.


SEQUENCE LISTING (781 sequences)

SEQ ID NO: 1 - DNA, 312 bp, Artificial sequence
  Descripition of the artificial sequence Sequence of the 5′-RACE fragment of Fig. 1A.
ggttcaagccgcctgagttgtgctgtcaccgctgccatgacttcgcgacccgtcactctcggtatcgacttgggcaccacgtccgtaaaggccgccctccatgcccgtggggttccagccaccgcagactccggatcctgcagccccagtcgccttcttcccttacttcgacaggacctacctaggcgtggcagcgtcactcaacggaggcaatgtgctggccacgtttgtccacatgcttgttcagtggatgacagacctaggttgcaaattgggccacagaggatctggaaaggatggaacaacgggcta

SEQ ID NO: 2 - DNA, 240 bp, Artificial sequence
  Description of the artificial sequence Sequence of the 5′-RACE fragment of Fig. 1B.
gccattgctgcaaatgtttccttgagtgccagagtatgcccagagcccatccctgccgtacgccaggggaggggcgaggaccctcacagaggcagggaggccggccactcttaccacacagcagcctggctctcccacaaagaacagctccaaggcacttgctccatttggggtgtgcctgcacctagctggttgcaaattgggccacagaggatctggaaaggatggaacaacgggcta

SEQ ID NO: 3 - DNA, 187 bp, Artificial sequence
  Description of the artificial sequence Sequence of the 5′-RACE fragment of Fig. 1C.
aggcagtgggtccctgagagaggtggtggtcagttggcttatgttgacttctgcagcaggagcgagagactgcagctgctgcagagcccagaggttgagcatctctggaggcctgtgcactggcttctccaagcagaggttgcaaattgggccacagaggatctggaaaggatggaacaacgggcta

SEQ ID NO: 4 - DNA, 160 bp, Rattus norvegicus
  The sequence section AC126839, position 87587 - 87746 (Fig. 2) is shown.
ctgctaagtgctcctctgccctggcatggctgggggtggggcattggctgtggtttctgaaaaagggcaaaaatgatgggaaaagctttgggatcctctgggaatcggagccgtggtaacagcagctgctgccattgctgcaaatgtttccttgagtgcc

SEQ ID NO: 5 - DNA, 160 bp, Mus musculus
  The sequence section AL663116, position 35875 - 36034 (Fig. 2) is shown.
cagctaagtgcacctcggccctggcatgactgggggtggggcattggctgtggtttctgaaaaagggcaaaaatgatgggaaaagctttgggatcctctgggaatcggagccgtggcaacagcagctgctgccattgctgcaaatgtttccttgagtgcc

SEQ ID NO: 6 - DNA, 165 bp, Homo sapiens
  The sequence section AF 168787, position 32580 - 32416 (Fig. 2) is shown.
ctggtaagtgctctgcagccctggcatggctggaggaggggcagcagctaagtggtttctgaaaaagggcaaaaatgatgggaaaagctttgggatcctctgggaatcagagccgcagcaaaggcagctgcttgcatcgctggaagcgttttttccttgagtgcc

SEQ ID NO: 7 - DNA, 1450 bp, Rattus norvegicus
  Genomic sequence in the 5′ region upstream of exon 1a of the VR1 gene of the rat (Fig. 3).
tgcccccatggacctaactgccaacaatccacaagagtggggagacccatattcctcaagggataacacagattggacaaagaatatgttccctggagatgaactatccagatgccatgtttcccatttacctgtatgattttggaatatttattaatctattttccctaatggggaaacagagtggtagtcccacagaattaaagttagatgaaatataaggcatacattctcaagggggttaaaaaataggttctggatgtctgaaaaatagctgtaataactgcttctctctctctttcccaaacgccataattaaaaaaaatagagcttaccagctgtattaatagttttcgatggattgataagataaaagataaaaagttgaaaaacaatgatgcaatcaatgttatttattactactatcacccagggctcccagcaagctctcacaaagcatagatagtcattcctcaacattattgaaaaggcaagacccaggtatcctctctttctgggagaatcaacagctcctggaacagtcctatcagcatcatctaacttcccccgaacccccaaagctacctgcacattcccaagacagattcaggcggtgccacaggaggtaagcaaggggctccaagagcactcgaatagtgccaccaacaccagccagtgtcagtacttttggtcaccaatactctggttctacaacctgggaatgtgcaggtctgggttggggctagaggctagaaacccaggcgctgctaaaagaggctagattcgcggcaggttgcccctcttaggtgcgaggctcatgtctgttggggtggctgtcccgatcgcgatgattctcacccgacagctttccaggcttggtggaccgacctcccaaaagcagcaatctcgcattagataccgcttaagaatgtaagggactctttgggcgccgctgactttgtgtgaaagcggggccagttgtcacctgacctgaaccacctgacttgaggctgagagccccgtgcaaggagctctggaagttgtagttttcaagaatcatcggcagctaacacttgatagggttgtgggaaaggtggaagggcggaagagctttggcaccttggcaaggacaggggctcggatgtgaggggagatctagtctcttgtaggggaggcagggctctcgaagtctctgttatatttctgaaaccctgtgctccttggtgaacaccctgggacgccaggcactgtcccggaacttctgaccataaggtcccgccttctgtcctctccacctatcactggaggccgctgcgaggagcatgtatgagtctgctttcctcgaccaataggagctgttaaaagattggcagggacgccccgtcccctcgctgccctccgggggtgtagttccttagcctgcggttcaagccgcctgagttgtgctgtc

SEQ ID NO: 8 - DNA, 4687 bp, Rattus norvegicus
  Genomic VR1 sequence of the rat in the 5′ direction of exon 1d (Fig. 4).
aaagacaattacttgttacatatacatacaattttacctttatcaaatcaaatgtgaatcctaactagacagatgggtttgtctaacaaaacccaaccaaacatactgatttgatatttatatgcgaaaaaatattaggaaaccatcccaagtctttgttttccataattctaccattatatttttatgtgcagacgactttgtatgcacaatcaaaacactgcagttcataacatacacatctgagccctggtagtttaggtcttggtcattggcattgtgtgctgtggaagggcacgcagttcaaagcaaacctggctgtgttcagaaccaaggctgaagtgaagttgtgcaatgtgacattcgcgggcaagcattgccagaagcacctcagctccattatgtacttgatgttgaactaagttttgagcttccagaaacctgttactattttgatttttttttctatcttcaaatttagtaatgtgacagtcatgtggagtttgcaatgcatagcttaagaagttttacttcacagcacctcaggatatcttggtttctggctgtgtcactatagcctcctacctcaggaacagccagctgtgggtggctcattcctgtaacctcagcacttctgatgcagaaacattaaggttgatacaagttcgaggccagcccggtttacatggcatgttccaggtcagccttggctatgtagcccgatcctacttcaaaaacaacaaacaaaaatcccagaggaactcaaacagagagaagcaatttcattcctagggcaaggatcaagagctcgtcttaagtcgatgtggctcctggtgcagccagggaacagtggccctggatagccttagtgacctctagcatctttggagcccgattttgctcagtcatttgtcagtagtagtgtgtctttcctctttccccattggccagagcttaggaaaacgccggtccccagaagtcatcccgagagcatgcttgacttccagggaggttaatgagcccctcatcacctagatccgtgcttcagcttgtggttcagagaacctcatcggttgctatgatgcttagatggaaggtttgtccttgccatctgtgctcagagcaggtgatattcaggtaaagccacaccagaccacagtctacaccagctgctttgggtaaggcacagcgcaggttcacagggatggggagtatgatggcttctattgacccaggccacttggagtctcagaagcaagtcatgaaaacgagaagttcaatctcaagctagcattaggaagctccagaggaggccagtgcagccataaccatgcaggtgatgtctttgtttcctttctatatgtgtatgatggggacagagtcatacccagtgggctagagagatggccgggtcaggaacagcacttgctattcgagcatgaggacttgagttcaagatcccaacagcctataaagagtcaggcaagagtgcatgtgccagcaaatctcacgcctgatatcctgctctgggtctggcaaatgcaagcataggcacatgcgctcacatgttcaggaacatgtatgcatgtacgcatacacacacacacacacacacacacacacacacacacacacacacacacacccctcatactcagctcatgaagcttttgtgagtccaatgagatagtatggatgaagcgtagataccaacctggaatgttctatgcctccagttaagtacccatggcactggaaccacagcgccacctgccaggaagtgtataggccgagttgccgagttttctgtaagagtttggaagtgatgggtttaggctgggctttagacagggagggcctgtgaggtaaggctgaatggcacaactgtgatgtctattgttcttacccggtaccccaagactgctaagtaagtggacaagatggacagtgcagtggcttgggcagaacgacggcggaggaggagtgtggaggcagcacagtgagactagatgaaaagcctcccggtccccgtgatgacagaagatgggtcccaggaaaagaggaaatgcgctatgggctaaagatgcagagctgaaggctggggaatctttcagcagtgtagaccatcaggaggctggggaaggaccaggatggttggtgatcagggagtagggatgagggacacttctgaccttccagataagagaaagggactggctggcaaacagaaggagggatgggagggaggcctagacattgagggcagacactcagagaaggagaggatgaggacaagagataatatcagattagaggaccagaagaagaagaggccttttataggggacggccagtcagggtgcttaattcagacagatcagagacagtcccgagacaagctgagagaaggatttgctgtccctatgtacagactcagggaggcagaggtggacctcagagagatacagggaagatgataagaaggggcagtggtggagagagagaagcagctgttggggacaaaggttcggaggcttgtttaagaaagagagtttcggggttggggatttggctcagtgacagagcgcttgcctaggaagcgcaaggccctgggttcggtccccagctccgaaaaaaagaaccaaaaaaaaaaaaagaaaaaaaaagaaaaaagaaagagagtttcagtttgcgaggtgaggaagctctgcagagatggtggctgcagatgccagttacatgaatgaacggaagatgacccaactatgcacttgaggtggttaggataaccaggtttaggctccctaggtcttatcaccatctaaaaatgagaacaggcccttggtgtcctgtgaatcgaagctaaaatggcatccatagtagaggagtgtggtaaggaatagtttgcttagcctggctctcatgcccggattaatcatgtgacgtgagtaactgtctagtacccaggtgccccaggtttctcctctgcaatttaagtatgacagtctttgctcacccctgcccatggttgttactgggcctgagagggaggtgtggagaagctctttggtagcacttttgcagagtggctgggagttcctctgctctaatgactccactctggtcccagcttttgtacttctccaggcggagctgccgtggctgctccactggagcagtgtctgaaaaaaaaaataaaaaggaaagaaaaggacatgactgtttttcggtgcggtggaagagaaagtttattgtagataaagggggagcatagacagaggcagacatgtctgggagagccagagtggttgtgaccctgagccatatggagaggtggggtgaggggtggcagagagggatcgagagaggagagaggggaaccagatgtagcagccaggaggccaaaggtacaaaaggggtgggtaaccaaaatgtctggattatataaaaaagagccagaggtcaggcccactttgatatgttaaataggcacctcagccatttatccaggtttgaaatgtaatataatttacatccccctggcttcctagagaccgttgtttagacggatgacctctgcagaatgtttgagggtgcagtcttgcatgtactccctggtgggctttcttgggcaggatctgggcaggaatgggcttgttctagtcacccactgcgtatgatggatgaacccgcttcctagtagttaggatggcactgggggaggcgagaaattagcacacgtaacgttttcttgtgttctattgttcactaagggaccccagtcaagcaagactgggccttggaagacctagagaccaccaaacctaatctctaccccgggtctgagtacacagggactcagagtcccaaagggggcagggcctccagacaggtggctcagaggtcccagtcctttggaaacatggcatcttcaggacactgggctttgcatctctggctgtgacagtcctttaagggagctactcctcagacatacaggagagatggtttggaaagtccgagatccaaagcctggttcaggctggactgggctgcaggctgctaagtgctcctctgccctggcatggctgggggtggggcattggctgtggtttctgaaaaagggcaaaaatgatgggaaaagctttgggatcctctgggaatcggagccgtggtaacagcagctgctgccattgctgcaaatgtttccttgagtgccagagtatgcccagagcccatccctgccgtacgccaggggaggggcgaggaccctcacagaggcagggaggccggccactcttaccacacagcagcctggctctcccacaaagaacagctccaaggcacttgctccatttggggtgtgcctgcacctagctggtaagtccttctcatggcctgatggctccccattgtccagctgtgtgtgcttctgctgggctctgagggcctcgattaccccatctgaaaactgggaggtacagcggctgcctcggggatttctgggaggctgaagcaccattactgcacacttaacttggctgtctgtaggcagtgggtccctgagagaggtggtggtcagttggcttatgttgacttctgcagcaggagcgagagactgcagctgctgcagagcccagaggttgagcatctctggaggcctgtgcactggcttctccaagcagag

SEQ ID NO: 9 - DNA, 27 bp, Artificial sequence
  Description of the artificial sequence Oligonucleotide AGW85 (gene-specific primer).
cctctgagtctaagctagcccgttgtt

SEQ ID NO: 10 - DNA, 25 bp, Artificial sequence
  Description of the artificial sequence Oligonucleotide rVR72 (gene-specific primer).
tagcccgttgttccatcctttccag

SEQ ID NO: 11 - DNA, 25 bp, Artificial sequence
  Description of the artificial sequence Primer VR1ab-35R.
cgagagtgacgggtcgcgaagtcat

SEQ ID NO: 12 - DNA, 25 bp, Artificial sequence
  Description of the artificial sequence Primer VR1ab-1R.
gacagcacaactcaggcggcttgaa

SEQ ID NO: 13 - DNA, 25 bp, Artificial sequence
  Description of the artificial sequence Primer AGW23.
cagctaggtgcaggcacaccccaaa

SEQ ID NO: 14 - DNA, 25 bp, Artificial sequence
  Description of the artificial sequence Primer AGW4.
cccaaatggagcaagtgccttggag

SEQ ID NO: 15 - DNA, 28 bp, Artificial sequence
  Description of the artificial sequence Primer AGWZ021.
tgtgagcgcatgtgcctatgcttgcatt

SEQ ID NO: 16 - DNA, 28 bp, Artificial sequence
  Description of the artificial sequence Primer AGWZ001.
cttgcatttgccagacccagagcaggat

SEQ ID NO: 17 - DNA, 20 bp, Artificial sequence
  Description of the artificial sequence Forward primer 1C-145F.
cagctccaaggcacttgctc

SEQ ID NO: 18 - DNA, 27 bp, Artificial sequence
  Description of the artificial sequence Forward primer VR1d-18F.
gagaggtggtggtcagttggcttatgt

SEQ ID NO: 19 - DNA, 20 bp, Artificial sequence
  Description of the artificial sequence Reverse primer 1c-417R.
gccagcccgccttcctcata

SEQ ID NO: 20 - DNA, 28 bp, Artificial sequence
  Description of the artificial sequence GAPDH primer.
cgaccccttcattgacctcaactacatg

SEQ ID NO: 21 - DNA, 28 bp, Artificial sequence
  Description of the artificial sequence GAPDH primer.
ccccggccttctccatggtggtgaagac

SEQ ID NO: 22 - DNA, 12 bp, Rattus norvegicus
  V$SRY 02
gccaacaatcca

SEQ ID NO: 23 - DNA, 10 bp, Rattus norvegicus
  V$SOX5 01
ccaacaatcc

SEQ ID NO: 24 - DNA, 8 bp, Rattus norvegicus
  V$MZF1 01
agtgggga

SEQ ID NO: 25 - DNA, 10 bp, Rattus norvegicus
  V$NFKAPPAB50 01
ggggagaccc

SEQ ID NO: 26 - DNA, 12 bp, Rattus norvegicus
  V$IK2 01
tcaagggataac

SEQ ID NO: 27 - DNA, 10 bp, Rattus norvegicus
  V$GATA1 05
agggataaca

SEQ ID NO: 28 - DNA, 10 bp, Rattus norvegicus
  V$GATA3 02
agggataaca

SEQ ID NO: 29 - DNA, 10 bp, Rattus norvegicus
  V$GATA2 02
agggataaca

SEQ ID NO: 30 - DNA, 9 bp, Rattus norvegicus
  V$LMO2COM 02
gggataaca

SEQ ID NO: 31 - DNA, 15 bp, Rattus norvegicus
  V$HNF3B 01
tggaatatttattaa

SEQ ID NO: 32 - DNA, 12 bp, Rattus norvegicus
  V$IK2 01
aatggggaaaca

SEQ ID NO: 33 - DNA, 8 bp, Rattus norvegicus
  V$MZF1 01
aatgggga

SEQ ID NO: 34 - DNA, 12 bp, Rattus norvegicus
  V$NFAT Q6
atggggaaacag

SEQ ID NO: 35 - DNA, 16 bp, Rattus norvegicus
  V$S8 01
cccacagaattaaagt

SEQ ID NO: 36 - DNA, 15 bp, Rattus norvegicus
  V$TST1 01
acagaattaaagtta

SEQ ID NO: 37 - DNA, 16 bp, Rattus norvegicus
  V$TH1E47 01
aataggttctggatgt

SEQ ID NO: 38 - DNA, 16 bp, Rattus norvegicus
  V$S8 01
acgccataattaaaaa

SEQ ID NO: 39 - DNA, 8 bp, Rattus norvegicus
  V$NKX25 02
cataatta

SEQ ID NO: 40 - DNA, 10 bp, Rattus norvegicus
  V$AP4 Q5
accagctgta

SEQ ID NO: 41 - DNA, 10 bp, Rattus norvegicus
  V$GATA1 06
attgataaga

SEQ ID NO: 42 - DNA, 10 bp, Rattus norvegicus
  V$GATA2 02
attgataaga

SEQ ID NO: 43 - DNA, 10 bp, Rattus norvegicus
  V$GATA3 02
attgataaga

SEQ ID NO: 44 - DNA, 9 bp, Rattus norvegicus
  V$LMO2COM 02
ttgataaga

SEQ ID NO: 45 - DNA, 11 bp, Rattus norvegicus
  V$EVI1 05
tgataagataa

SEQ ID NO: 46 - DNA, 11 bp, Rattus norvegicus
  V$EVI1 03
tgataagataa

SEQ ID NO: 47 - DNA, 11 bp, Rattus norvegicus
  V$EVI1 02
tgataagataa

SEQ ID NO: 48 - DNA, 13 bp, Rattus norvegicus
  V$GATA1 04
ataagataaaaga

SEQ ID NO: 49 - DNA, 10 bp, Rattus norvegicus
  V$GATA2 02
taagataaaa

SEQ ID NO: 50 - DNA, 10 bp, Rattus norvegicus
  V$GATA3 02
taagataaaa

SEQ ID NO: 51 - DNA, 9 bp, Rattus norvegicus
  V$LMO2COM 02
aagataaaa

SEQ ID NO: 52 - DNA, 10 bp, Rattus norvegicus
  V$GATA1 06
aaagataaaa

SEQ ID NO: 53 - DNA, 10 bp, Rattus norvegicus
  V$GATA2 02
aaagataaaa

SEQ ID NO: 54 - DNA, 10 bp, Rattus norvegicus
  V$GATA3 02
aaagataaaa

SEQ ID NO: 55 - DNA, 9 bp, Rattus norvegicus
  V$LMO2COM 02
aagataaaa

SEQ ID NO: 56 - DNA, 12 bp, Rattus norvegicus
  V$SRY 02
aaaaacaatgat

SEQ ID NO: 57 - DNA, 10 bp, Rattus norvegicus
  V$SOX5 01
aaaacaatga

SEQ ID NO: 58 - DNA, 14 bp, Rattus norvegicus
  V$CEBPB 01
caatgatgcaatca

SEQ ID NO: 59 - DNA, 24 bp, Rattus norvegicus
  V$GFI1 01
aatgatgcaatcaatgttatttat

SEQ ID NO: 60 - DNA, 14 bp, Rattus norvegicus
  V$GATA1 02
gcatagatagtcat

SEQ ID NO: 61 - DNA, 9 bp, Rattus norvegicus
  V$LMO2COM 02
tagatagtc

SEQ ID NO: 62 - DNA, 13 bp, Rattus norvegicus
  V$TCF11 01
gtcattcctcaac

SEQ ID NO: 63 - DNA, 11 bp, Rattus norvegicus
  V$CP2 01
gcaagacccag

SEQ ID NO: 64 - DNA, 12 bp, Rattus norvegicus
  V$IK2 01
ttctgggagaat

SEQ ID NO: 65 - DNA, 10 bp, Rattus norvegicus
  V$AP4 Q5
aacagctcct

SEQ ID NO: 66 - DNA, 11 bp, Rattus norvegicus
  V$NFY Q6
tcaccaatact

SEQ ID NO: 67 - DNA, 13 bp, Rattus norvegicus
  V$IK1 01
acctgggaatgtg

SEQ ID NO: 68 - DNA, 12 bp, Rattus norvegicus
  V$IK2 01
acctgggaatgt

SEQ ID NO: 69 - DNA, 18 bp, Rattus norvegicus
  V$CMYB 01
gctcatgtctgttggggt

SEQ ID NO: 70 - DNA, 14 bp, Rattus norvegicus
  V$GATA1 02
cattagataccgct

SEQ ID NO: 71 - DNA, 9 bp, Rattus norvegicus
  V$LMO2COM 02
tagataccg

SEQ ID NO: 72 - DNA, 11 bp, Rattus norvegicus
  V$AP1 Q2
gctgactttgt

SEQ ID NO: 73 - DNA, 18 bp, Rattus norvegicus
  V$CMYB 01
agcggggccagttgtcac

SEQ ID NO: 74 - DNA, 11 bp, Rattus norvegicus
  V$SREBP1 01
tgtcacctgac

SEQ ID NO: 75 - DNA, 11 bp, Rattus norvegicus
  V$DELTAEF1 01
tgtcacctgac

SEQ ID NO: 76 - DNA, 10 bp, Rattus norvegicus
  V$MYOD Q6
gtcacctgac

SEQ ID NO: 77 - DNA, 11 bp, Rattus norvegicus
  V$DELTAEF1 01
aaccacctgac

SEQ ID NO: 78 - DNA, 10 bp, Rattus norvegicus
  V$MYOD Q6
accacctgac

SEQ ID NO: 79 - DNA, 24 bp, Rattus norvegicus
  V$GFI1 01
ttttcaagaatcatcggcagctaa

SEQ ID NO: 80 - DNA, 14 bp, Rattus norvegicus
  V$GATA1 02
cacttgatagggtt

SEQ ID NO: 81 - DNA, 9 bp, Rattus norvegicus
  V$LMO2COM 02
ttgataggg

SEQ ID NO: 82 - DNA, 13 bp, Rattus norvegicus
  V$IK1 01
ttgtgggaaaggt

SEQ ID NO: 83 - DNA, 12 bp, Rattus norvegicus
  V$IK2 01
ttgtgggaaagg

SEQ ID NO: 84 - DNA, 12 bp, Rattus norvegicus
  V$NFAT Q6
tgtgggaaaggt

SEQ ID NO: 85 - DNA, 14 bp, Rattus norvegicus
  V$GKLF 01
gaaaggtggaaggg

SEQ ID NO: 86 - DNA, 10 bp, Rattus norvegicus
  V$NRF2 01
ggcggaagag

SEQ ID NO: 87 - DNA, 18 bp, Rattus norvegicus
  V$NF1 Q6
ctttggcaccttggcaag

SEQ ID NO: 88 - DNA, 11 bp, Rattus norvegicus
  V$DELTAEF1 01
tggcaccttgg

SEQ ID NO: 89 - DNA, 18 bp, Rattus norvegicus
  V$NF1 Q6
ccttggcaaggacagggg

SEQ ID NO: 90 - DNA, 8 bp, Rattus norvegicus
  V$MZF1 01
tgagggga

SEQ ID NO: 91 - DNA, 8 bp, Rattus norvegicus
  V$MZF1 01
gtagggga

SEQ ID NO: 92 - DNA, 12 bp, Rattus norvegicus
  V$IK2 01
ccctgggacgcc

SEQ ID NO: 93 - DNA, 10 bp, Rattus norvegicus
  V$CETS1P54 01
cccggaactt

SEQ ID NO: 94 - DNA, 11 bp, Rattus norvegicus
  V$DELTAEF1 01
ctccacctatc

SEQ ID NO: 95 - DNA, 16 bp, Rattus norvegicus
  V$NFY 01
ctcgaccaataggagc

SEQ ID NO: 96 - DNA, 18 bp, Rattus norvegicus
  V$NF1 Q6
gattggcagggacgcccc

SEQ ID NO: 97 - DNA, 12 bp, Rattus norvegicus
  V$IK2 01
ggcagggacgcc

SEQ ID NO: 98 - DNA, 8 bp, Mus musculus
  V$NKX25 02
cctaattg

SEQ ID NO: 99 - DNA, 18 bp, Mus musculus
  V$NF1 Q6
aattggccataatccatg

SEQ ID NO: 100 - DNA, 8 bp, Mus musculus
  V$MZF1 01
agtgggga

SEQ ID NO: 101 - DNA, 10 bp, Mus musculus
  V$NFKAPPAB50 01
ggggagaccc

SEQ ID NO: 102 - DNA, 14 bp, Mus musculus
  V$GATA1 02
caagggatagtaca

SEQ ID NO: 103 - DNA, 16 bp, Mus musculus
  V$TH1E47 01
gtatgattctggaata

SEQ ID NO: 104 - DNA, 8 bp, Mus musculus
  V$NKX25 02
cttaatgg

SEQ ID NO: 105 - DNA, 12 bp, Mus musculus
  V$IK2 01
aatggggaaaca

SEQ ID NO: 106 - DNA, 8 bp, Mus musculus
  V$MZF1 01
aatgggga

SEQ ID NO: 107 - DNA, 12 bp, Mus musculus
  V$NFAT Q6
atggggaaacag

SEQ ID NO: 108 - DNA, 13 bp, Mus musculus
  V$TCF11 01
gtcataataaaaa

SEQ ID NO: 109 - DNA, 10 bp, Mus musculus
  V$AP4 Q5
accagctgta

SEQ ID NO: 110 - DNA, 15 bp, Mus musculus
  V$BARBIE 01
attaaaagtttgagg

SEQ ID NO: 111 - DNA, 10 bp, Mus musculus
  V$GATA1 05
taagataaca

SEQ ID NO: 112 - DNA, 10 bp, Mus musculus
  V$GATA2 02
taagataaca

SEQ ID NO: 113 - DNA, 10 bp, Mus musculus
  V$GATA3 02
taagataaca

SEQ ID NO: 114 - DNA, 9 bp, Mus musculus
  V$LMO2COM 02
aagataaca

SEQ ID NO: 115 - DNA, 12 bp, Mus musculus
  V$SRY 02
gataacaaaaag

SEQ ID NO: 116 - DNA, 12 bp, Mus musculus
  V$SRY 02
agaaacaatgag

SEQ ID NO: 117 - DNA, 10 bp, Mus musculus
  V$SOX5 01
gaaacaatga

SEQ ID NO: 118 - DNA, 12 bp, Mus musculus
  V$HFH2 01
gattgttatttt

SEQ ID NO: 119 - DNA, 11 bp, Mus musculus
  V$CP2 01
gcaagatccag

SEQ ID NO: 120 - DNA, 12 bp, Mus musculus
  V$IK2 01
ctctgggagaat

SEQ ID NO: 121 - DNA, 10 bp, Mus musculus
  V$AP4 Q5
accagctgtc

SEQ ID NO: 122 - DNA, 18 bp, Mus musculus
  V$CMYB 01
gctaatgtctgttggggt

SEQ ID NO: 123 - DNA, 9 bp, Mus musculus
  V$PADS C
ggtggtgtc

SEQ ID NO: 124 - DNA, 13 bp, Mus musculus
  V$TCF11 01
gtcataccagaaa

SEQ ID NO: 125 - DNA, 11 bp, Mus musculus
  V$DELTAEF1 01
tctcacctgac

SEQ ID NO: 126 - DNA, 10 bp, Mus musculus
  V$MYOD Q6
ctcacctgac

SEQ ID NO: 127 - DNA, 11 bp, Mus musculus
  V$AP1FJ Q2
cctgacagctt

SEQ ID NO: 128 - DNA, 10 bp, Mus musculus
  V$SOX5 01
gcaacaatct

SEQ ID NO: 129 - DNA, 10 bp, Mus musculus
  V$AP4 Q5
gccagctgtc

SEQ ID NO: 130 - DNA, 11 bp, Mus musculus
  V$SREBP1 01
tgtcacctgac

SEQ ID NO: 131 - DNA, 11 bp, Mus musculus
  V$DELTAEF1 01
tgtcacctgac

SEQ ID NO: 132 - DNA, 10 bp, Mus musculus
  V$MYOD Q6
gtcacctgac

SEQ ID NO: 133 - DNA, 11 bp, Mus musculus
  V$DELTAEF1 01
aaccacctgac

SEQ ID NO: 134 - DNA, 10 bp, Mus musculus
  V$MYOD Q6
accacctgac

SEQ ID NO: 135 - DNA, 11 bp, Mus musculus
  V$AP1FJ Q2
cctgacttgaa

SEQ ID NO: 136 - DNA, 14 bp, Mus musculus
  V$GATA1 03
aggaggataacgct

SEQ ID NO: 137 - DNA, 10 bp, Mus musculus
  V$GATA2 02
gaggataacg

SEQ ID NO: 138 - DNA, 9 bp, Mus musculus
  V$LMO2COM 02
aggataacg

SEQ ID NO: 139 - DNA, 12 bp, Mus musculus
  V$IK2 01
gtgagggaaggg

SEQ ID NO: 140 - DNA, 14 bp, Mus musculus
  V$GKLF 01
gaagggtggaaggg

SEQ ID NO: 141 - DNA, 10 bp, Mus musculus
  V$NRF2 01
ggcggaagag

SEQ ID NO: 142 - DNA, 11 bp, Mus musculus
  V$DELTAEF1 01
aggcaccttgg

SEQ ID NO: 143 - DNA, 18 bp, Mus musculus
  V$NF1 Q6
ccttggcagggacagggg

SEQ ID NO: 144 - DNA, 8 bp, Mus musculus
  V$MZF1 01
gtagggga

SEQ ID NO: 145 - DNA, 12 bp, Mus musculus
  V$IK2 01
gtaggggaagca

SEQ ID NO: 146 - DNA, 12 bp, Mus musculus
  V$IK2 01
ccctgggacccc

SEQ ID NO: 147 - DNA, 10 bp, Mus musculus
  V$CETS1P54 01
cccggaacct

SEQ ID NO: 148 - DNA, 11 bp, Mus musculus
  V$AP1FJ Q2
tctgaccaata

SEQ ID NO: 149 - DNA, 11 bp, Mus musculus
  V$NFY Q6
tgaccaataga

SEQ ID NO: 150 - DNA, 10 bp, Mus musculus
  V$CDPCR3HD 01
aatagatccc

SEQ ID NO: 151 - DNA, 11 bp, Mus musculus
  V$DELTAF1 01
ctccacctatc

SEQ ID NO: 152 - DNA, 10 bp, Mus musculus
  V$CETS1P54 01
accggaggcc

SEQ ID NO: 153 - DNA, 8 bp, Mus musculus
  V$MZF1 01
tgtgggga

SEQ ID NO: 154 - DNA, 16 bp, Mus musculus
  V$AHRARNT 01
tggggagcgcgtggtg

SEQ ID NO: 155 - DNA, 9 bp, Mus musculus
  V$PADS C
cgtggtgtt

SEQ ID NO: 156 - DNA, 15 bp, Mus musculus
  V$HNF3B 01
cgtggtgtttgcttt

SEQ ID NO: 157 - DNA, 13 bp, Mus musculus
  V$HFH3 01
tggtgtttgcttt

SEQ ID NO: 158 - DNA, 16 bp, Mus musculus
  V$NFY 01
ctcgaccaatagaagt

SEQ ID NO: 159 - DNA, 7 bp, Mus musculus
  V$NKX25 01
tgaagtg

SEQ ID NO: 160 - DNA, 11 bp, Homo sapiens
  V$DELTAEF1 01
acccacctgac

SEQ ID NO: 161 - DNA, 10 bp, Homo sapiens
  V$MYOD Q6
cccacctgac

SEQ ID NO: 162 - DNA, 8 bp, Homo sapiens
  V$MZF1 01
ctggggga

SEQ ID NO: 163 - DNA, 11 bp, Homo sapiens
  V$DELTAEF1 01
aggcacctgcc

SEQ ID NO: 164 - DNA, 10 bp, Homo sapiens
  V$MYOD Q6
ggcacctgcc

SEQ ID NO: 165 - DNA, 10 bp, Homo sapiens
  V$AP4 Q5
accagctggc

SEQ ID NO: 166 - DNA, 19 bp, Homo sapiens
  V$ER Q6
gctggcctcagtgaccaga

SEQ ID NO: 167 - DNA, 11 bp, Homo sapiens
  V$AP1FJ Q2
agtgaccagaa

SEQ ID NO: 168 - DNA, 16 bp, Homo sapiens
  V$ARNT 01
tggggcacgtgacccg

SEQ ID NO: 169 - DNA, 14 bp, Homo sapiens
  V$MAX 01
ggggcacgtgaccc

SEQ ID NO: 170 - DNA, 14 bp, Homo sapiens
  V$USF 01
ggggcacgtgaccc

SEQ ID NO: 171 - DNA, 12 bp, Homo sapiens
  V$NMYC 01
gggcacgtgacc

SEQ ID NO: 172 - DNA, 12 bp, Homo sapiens
  V$MYCMAX 02
gggcacgtgacc

SEQ ID NO: 173 - DNA, 10 bp, Homo sapiens
  V$USF Q6
ggcacgtgac

SEQ ID NO: 174 - DNA, 8 bp, Homo sapiens
  V$USF C
gcacgtga

SEQ ID NO: 175 - DNA, 11 bp, Homo sapiens
  V$AP1FJ Q2
cgtgacccggg

SEQ ID NO: 176 - DNA, 10 bp, Homo sapiens
  V$AP4 Q5
ttcagcagct

SEQ ID NO: 177 - DNA, 10 bp, Homo sapiens
  V$AP4 Q5
agcagctcca

SEQ ID NO: 178 - DNA, 12 bp, Homo sapiens
  V$IK2 01
cagtgggagtgc

SEQ ID NO: 179 - DNA, 12 bp, Homo sapiens
  V$CREB 02
ggaatgacgtgc

SEQ ID NO: 180 - DNA, 17 bp, Homo sapiens
  V$XBP1 01
ggaatgacgtgctgaag

SEQ ID NO: 181 - DNA, 8 bp, Homo sapiens
  V$CREB 01
tgacgtgc

SEQ ID NO: 182 - DNA, 10 bp, Homo sapiens
  V$CREL 01
agggctttcc

SEQ ID NO: 183 - DNA, 10 bp, Homo sapiens
  V$NFKAPPAB65 01
agggctttcc

SEQ ID NO: 184 - DNA, 15 bp, Homo sapiens
  V$E47 01
gatgcagctgtcggg

SEQ ID NO: 185 - DNA, 10 bp, Homo sapiens
  V$AP4 Q5
tgcagctgtc

SEQ ID NO: 186 - DNA, 10 bp, Homo sapiens
  V$AP4 Q5
ggcagctctg

SEQ ID NO: 187 - DNA, 13 bp, Homo sapiens
  V$TCF11 01
gtcatgctccgga

SEQ ID NO: 188 - DNA, 11 bp, Homo sapiens
  V$DELTAEF1 01
agacacctcaa

SEQ ID NO: 189 - DNA, 11 bp, Homo sapiens
  V$AP1FJ Q2
cctgacaccat

SEQ ID NO: 190 - DNA, 13 bp, Homo sapiens
  V$TCF11 01
gtcatcctttccc

SEQ ID NO: 191 - DNA, 16 bp, Homo sapiens
  V$BRN2 01
cagatcccaaatgagt

SEQ ID NO: 192 - DNA, 11 bp, Homo sapiens
  V$AP1FJ Q2
attgacccacc

SEQ ID NO: 193 - DNA, 11 bp, Homo sapiens
  V$DELTAEF1 01
acccacctggg

SEQ ID NO: 194 - DNA, 10 bp, Homo sapiens
  V$MYOD Q6
cccacctggg

SEQ ID NO: 195 - DNA, 12 bp, Homo sapiens
  V$IK2 01
acctgggagcta

SEQ ID NO: 196 - DNA, 12 bp, Homo sapiens
  V$IK2 01
atgtgggagaga

SEQ ID NO: 197 - DNA, 10 bp, Homo sapiens
  V$AP4 Q5
gtcagcaggc

SEQ ID NO: 198 - DNA, 11 bp, Homo sapiens
  V$DELTAEF1 01
gttcacctgta

SEQ ID NO: 199 - DNA, 10 bp, Homo sapiens
  V$MYOD Q6
ttcacctgta

SEQ ID NO: 200 - DNA, 7 bp, Homo sapiens
  V$NKX25 01
ccaagtg

SEQ ID NO: 201 - DNA, 13 bp, Homo sapiens
  V$IK1 01
aagtgggaaaaga

SEQ ID NO: 202 - DNA, 12 bp, Homo sapiens
  V$IK2 01
aagtgggaaaag

SEQ ID NO: 203 - DNA, 12 bp, Homo sapiens
  V$NFAT Q6
agtgggaaaaga

SEQ ID NO: 204 - DNA, 12 bp, Homo sapiens
  V$NFAT Q6
ttctggaaaagt

SEQ ID NO: 205 - DNA, 10 bp, Homo sapiens
  V$CETS1P54 01
accggaggcc

SEQ ID NO: 206 - DNA, 12 bp, Homo sapiens
  V$IK2 01
gccagggattga

SEQ ID NO: 207 - DNA, 11 bp, Homo sapiens
  V$AP1FJ Q2
attgacccaag

SEQ ID NO: 208 - DNA, 11 bp, Homo sapiens
  V$CP2 01
gctgcacccag

SEQ ID NO: 209 - DNA, 8 bp, Homo sapiens
  V$MZF1 01
aaggggga

SEQ ID NO: 210 - DNA, 12 bp, Homo sapiens
  V$IK2 01
tcatgggaaggg

SEQ ID NO: 211 - DNA, 12 bp, Homo sapiens
  V$IK2 01
ggaagggatgca

SEQ ID NO: 212 - DNA, 16 bp, Homo sapiens
  V$AHRARNT 01
ttcgcttggcgtgggc

SEQ ID NO: 213 - DNA, 18 bp, Homo sapiens
  V$NF1 Q6
gcttggcgtgggctttgc

SEQ ID NO: 214 - DNA, 10 bp, Homo sapiens
  V$AP4 Q5
ctcagcagaa

SEQ ID NO: 215 - DNA, 18 bp, Homo sapiens
  V$NF1 Q6
agttggcatccctgtagg

SEQ ID NO: 216 - DNA, 12 bp, Homo sapiens
  V$IK2 01
tgtagggatccc

SEQ ID NO: 217 - DNA, 12 bp, Homo sapiens
  V$IK2 01
ggtagggatggc

SEQ ID NO: 218 - DNA, 10 bp, Rattus norvegicus
  V$VBP 01
gttacatata

SEQ ID NO: 219 - DNA, 24 bp, Rattus norvegicus
  V$GFI1 01
ctttatcaaatcaaatgtgaatcc

SEQ ID NO: 220 - DNA, 12 bp, Rattus norvegicus
  V$SRY 02
tctaacaaaacc

SEQ ID NO: 221 - DNA, 14 bp, Rattus norvegicus
  V$OCT1 06
gatatttatatgcg

SEQ ID NO: 222 - DNA, 12 bp, Rattus norvegicus
  V$NFAT Q6
attaggaaacca

SEQ ID NO: 223 - DNA, 8 bp, Rattus norvegicus
  V$NKX25 02
cataattc

SEQ ID NO: 224 - DNA, 24 bp, Rattus norvegicus
  V$GFI1 01
gtatgcacaatcaaaacactgcag

SEQ ID NO: 225 - DNA, 13 bp, Rattus norvegicus
  V$TCF11 01
gtcattggcattg

SEQ ID NO: 226 - DNA, 18 bp, Rattus norvegicus
  V$NF1 Q6
cattggcattgtgtgctg

SEQ ID NO: 227 - DNA, 7 bp, Rattus norvegicus
  V$NKX25 01
tgaagtg

SEQ ID NO: 228 - DNA, 14 bp, Rattus norvegicus
  V$CEBPB 01
aagttgtgcaatgt

SEQ ID NO: 229 - DNA, 11 bp, Rattus norvegicus
  V$DELTAEF1 01
aagcacctcag

SEQ ID NO: 230 - DNA, 10 bp, Rattus norvegicus
  V$AP4 Q5
ctcagctcca

SEQ ID NO: 231 - DNA, 13 bp, Rattus norvegicus
  V$TCF11 01
gtcatgtggagtt

SEQ ID NO: 232 - DNA, 11 bp, Rattus norvegicus
  V$DELTAEF1 01
cagcacctcag

SEQ ID NO: 233 - DNA, 16 bp, Rattus norvegicus
  V$THIE4/ 01
cttggtttctggctgt

SEQ ID NO: 234 - DNA, 13 bp, Rattus norvegicus
  V$TCF11 01
gtcactatagcct

SEQ ID NO: 235 - DNA, 10 bp, Rattus norvegicus
  V$AP4 Q5
gccagctgtg

SEQ ID NO: 236 - DNA, 9 bp, Rattus norvegicus
  V$STAT 01
ttcctgtaa

SEQ ID NO: 237 - DNA, 14 bp, Rattus norvegicus
  V$GATA1 03
aggttgatacaagt

SEQ ID NO: 238 - DNA, 13 bp, Rattus norvegicus
  V$RORA1 01
tgttccaggtcag

SEQ ID NO: 239 - DNA, 18 bp, Rattus norvegicus
  V$NF1 Q6
ccttggctatgtagcccg

SEQ ID NO: 240 - DNA, 12 bp, Rattus norvegicus
  V$SRY 02
aaaaacaacaaa

SEQ ID NO: 241 - DNA, 12 bp, Rattus norvegicus
  V$SRY 02
acaaacaaaaat

SEQ ID NO: 242 - DNA, 24 bp, Rattus norvegicus
  V$GFI1 01
caaacaaaaatcccagaggaactc

SEQ ID NO: 243 - DNA, 12 bp, Rattus norvegicus
  V$IK2 01
gccagggaacag

SEQ ID NO: 244 - DNA, 14 bp, Rattus norvegicus
  V$GATA1 02
ccctggatagcctt

SEQ ID NO: 245 - DNA, 9 bp, Rattus norvegicus
  V$LMO2COM 02
tggatagcc

SEQ ID NO: 246 - DNA, 11 bp, Rattus norvegicus
  V$AP1FJ Q2
agtgacctcta

SEQ ID NO: 247 - DNA, 13 bp, Rattus norvegicus
  V$TCF11 01
gtcatttgtcagt

SEQ ID NO: 248 - DNA, 18 bp, Rattus norvegicus
  V$NF1 Q6
cattggccagagcttagg

SEQ ID NO: 249 - DNA, 12 bp, Rattus norvegicus
  V$NFAT Q6
cttaggaaaacg

SEQ ID NO: 250 - DNA, 13 bp, Rattus norvegicus
  V$TCF11 01
gtcatcccgagag

SEQ ID NO: 251 - DNA, 11 bp, Rattus norvegicus
  V$AP1FJ Q2
cttgacttcca

SEQ ID NO: 252 - DNA, 11 bp, Rattus norvegicus
  V$DELTAEF1 01
catcacctaga

SEQ ID NO: 253 - DNA, 16 bp, Rattus norvegicus
  V$E47 02
cagagcaggtgatatt

SEQ ID NO: 254 - DNA, 12 bp, Rattus norvegicus
  V$MYOD 01
gagcaggtgata

SEQ ID NO: 255 - DNA, 12 bp, Rattus norvegicus
  V$LMO2COM 01
gagcaggtgata

SEQ ID NO: 256 - DNA, 13 bp, Rattus norvegicus
  V$GATA1 04
aggtgatattcag

SEQ ID NO: 257 - DNA, 10 bp, Rattus norvegicus
  V$AP4 Q5
accagctgct

SEQ ID NO: 258 - DNA, 12 bp, Rattus norvegicus
  V$IK2 01
cacagggatggg

SEQ ID NO: 259 - DNA, 8 bp, Rattus norvegicus
  V$MZF1 01
gatgggga

SEQ ID NO: 260 - DNA, 11 bp, Rattus norvegicus
  V$AP1FJ Q2
attgacccagg

SEQ ID NO: 261 - DNA, 13 bp, Rattus norvegicus
  V$TCF11 01
gtcatgaaaacga

SEQ ID NO: 262 - DNA, 16 bp, Rattus norvegicus
  V$E47 02
ccatgcaggtgatgtc

SEQ ID NO: 263 - DNA, 12 bp, Rattus norvegicus
  V$MYOD 01
atgcaggtgatg

SEQ ID NO: 264 - DNA, 12 bp, Rattus norvegicus
  V$LMO2COM 01
atgcaggtgatg

SEQ ID NO: 265 - DNA, 15 bp, Rattus norvegicus
  V$HNF3B 01
gtctttgtttccttt

SEQ ID NO: 266 - DNA, 8 bp, Rattus norvegicus
  V$MZF1 01
gatgggga

SEQ ID NO: 267 - DNA, 12 bp, Rattus norvegicus
  V$IK2 01
gatggggacaga

SEQ ID NO: 268 - DNA, 13 bp, Rattus norvegicus
  V$TCF11 01
gtcatacccagtg

SEQ ID NO: 269 - DNA, 15 bp, Rattus norvegicus
  V$TATA 01
ctataaagagtcagg

SEQ ID NO: 270 - DNA, 13 bp, Rattus norvegicus
  V$GATA1 04
gcctgatatcctg

SEQ ID NO: 271 - DNA, 9 bp, Rattus norvegicus
  V$LMO2COM 02
ctgatatcc

SEQ ID NO: 272 - DNA, 16 bp, Rattus norvegicus
  V$TH1E47 01
ctctgggtctggcaaa

SEQ ID NO: 273 - DNA, 10 bp, Rattus norvegicus
  V$AP4 Q5
ctcagctcat

SEQ ID NO: 274 - DNA, 12 bp, Rattus norvegicus
  V$CAAT 01
tgagtccaatga

SEQ ID NO: 275 - DNA, 16 bp, Rattus norvegicus
  V$NFY 01
tgagtccaatgagata

SEQ ID NO: 276 - DNA, 11 bp, Rattus norvegicus
  V$NFY Q6
agtccaatgag

SEQ ID NO: 277 - DNA, 14 bp, Rattus norvegicus
  V$GATA1 02
aatgagatagtatg

SEQ ID NO: 278 - DNA, 10 bp, Rattus norvegicus
  V$GATA2 03
tgagatagta

SEQ ID NO: 279 - DNA, 9 bp, Rattus norvegicus
  V$LMO2COM 02
gagatagta

SEQ ID NO: 280 - DNA, 14 bp, Rattus norvegicus
  V$GATA1 03
gcgtagataccaac

SEQ ID NO: 281 - DNA, 9 bp, Rattus norvegicus
  V$LMO2COM 02
tagatacca

SEQ ID NO: 282 - DNA, 11 bp, Rattus norvegicus
  V$DELTAEF1 01
cgccacctgcc

SEQ ID NO: 283 - DNA, 10 bp, Rattus norvegicus
  V$MYOD Q6
gccacctgcc

SEQ ID NO: 284 - DNA, 12 bp, Rattus norvegicus
  V$IK2 01
gacagggagggc

SEQ ID NO: 285 - DNA, 7 bp, Rattus norvegicus
  V$NKX25 01
gtaagtg

SEQ ID NO: 286 - DNA, 11 bp, Rattus norvegicus
  V$AP1FJ Q2
gatgacagaag

SEQ ID NO: 287 - DNA, 12 bp, Rattus norvegicus
  V$NFAT Q6
cccaggaaaaga

SEQ ID NO: 288 - DNA, 12 bp, Rattus norvegicus
  V$NFAT Q6
aagaggaaatgc

SEQ ID NO: 289 - DNA, 13 bp, Rattus norvegicus
  V$IK1 01
gctggggaatctt

SEQ ID NO: 290 - DNA, 12 bp, Rattus norvegicus
  V$IK2 01
gctggggaatct

SEQ ID NO: 291 - DNA, 8 bp, Rattus norvegicus
  V$MZF1 01
gctgggga

SEQ ID NO: 292 - DNA, 10 bp, Rattus norvegicus
  V$AP4 Q5
ttcagcagtg

SEQ ID NO: 293 - DNA, 8 bp, Rattus norvegicus
  V$MZF1 01
gctgggga

SEQ ID NO: 294 - DNA, 12 bp, Rattus norvegicus
  V$IK2 01
gctggggaagga

SEQ ID NO: 295 - DNA, 13 bp, Rattus norvegicus
  V$IK1 01
gctggggaaggac

SEQ ID NO: 296 - DNA, 12 bp, Rattus norvegicus
  V$IK2 01
agtagggatgag

SEQ ID NO: 297 - DNA, 10 bp, Rattus norvegicus
  V$GATA1 06
ccagataaga

SEQ ID NO: 298 - DNA, 10 bp, Rattus norvegicus
  V$GATA2 02
ccagataaga

SEQ ID NO: 299 - DNA, 10 bp, Rattus norvegicus
  V$GATA3 02
ccagataaga

SEQ ID NO: 300 - DNA, 9 bp, Rattus norvegicus
  V$LMO2COM 02
cagataaga

SEQ ID NO: 301 - DNA, 11 bp, Rattus norvegicus
  V$EVI1 02
agataagagaa

SEQ ID NO: 302 - DNA, 14 bp, Rattus norvegicus
  V$GKLF 01
gataagagaaaggg

SEQ ID NO: 303 - DNA, 14 bp, Rattus norvegicus
  V$GKLF 01
aaacagaaggaggg

SEQ ID NO: 304 - DNA, 12 bp, Rattus norvegicus
  V$IK2 01
aggagggatggg

SEQ ID NO: 305 - DNA, 12 bp, Rattus norvegicus
  V$IK2 01
ggatgggaggga

SEQ ID NO: 306 - DNA, 13 bp, Rattus norvegicus
  V$GATA1 04
aagagataatatc

SEQ ID NO: 307 - DNA, 10 bp, Rattus norvegicus
  V$GATA2 03
agagataata

SEQ ID NO: 308 - DNA, 10 bp, Rattus norvegicus
  V$GATA3 02
agagataata

SEQ ID NO: 309 - DNA, 9 bp, Rattus norvegicus
  V$LMO2COM 02
gagataata

SEQ ID NO: 310 - DNA, 8 bp, Rattus norvegicus
  V$MZF1 01
atagggga

SEQ ID NO: 311 - DNA, 8 bp, Rattus norvegicus
  V$NKX25 02
cttaattc

SEQ ID NO: 312 - DNA, 10 bp, Rattus norvegicus
  V$GATA3 03
acagatcaga

SEQ ID NO: 313 - DNA, 14 bp, Rattus norvegicus
  V$GATA1 02
agagagatacaggg

SEQ ID NO: 314 - DNA, 9 bp, Rattus norvegicus
  V$LMO2COM 02
gagatacag

SEQ ID NO: 315 - DNA, 12 bp, Rattus norvegicus
  V$IK2 01
tacagggaagat

SEQ ID NO: 316 - DNA, 10 bp, Rattus norvegicus
  V$GATA3 03
gaagatgata

SEQ ID NO: 317 - DNA, 14 bp, Rattus norvegicus
  V$GATA1 03
aagatgataagaag

SEQ ID NO: 318 - DNA, 10 bp, Rattus norvegicus
  V$GATA2 02
gatgataaga

SEQ ID NO: 319 - DNA, 10 bp, Rattus norvegicus
  V$GATA3 02
gatgataaga

SEQ ID NO: 320 - DNA, 14 bp, Rattus norvegicus
  V$GKLF 01
gatgataagaaggg

SEQ ID NO: 321 - DNA, 9 bp, Rattus norvegicus
  V$LMO2COM 02
atgataaga

SEQ ID NO: 322 - DNA, 18 bp, Rattus norvegicus
  V$CMYB 01
agaagcagctgttgggga

SEQ ID NO: 323 - DNA, 15 bp, Rattus norvegicus
  V$E47 01
gaagcagctgttggg

SEQ ID NO: 324 - DNA, 10 bp, Rattus norvegicus
  V$AP4 Q5
agcagctgtt

SEQ ID NO: 325 - DNA, 12 bp, Rattus norvegicus
  V$IK2 01
gttggggacaaa

SEQ ID NO: 326 - DNA, 8 bp, Rattus norvegicus
  V$MZF1 01
gttgggga

SEQ ID NO: 327 - DNA, 12 bp, Rattus norvegicus
  V$IK2 01
gttggggatttg

SEQ ID NO: 328 - DNA, 8 bp, Rattus norvegicus
  V$MZF1 01
gttgggga

SEQ ID NO: 329 - DNA, 18 bp, Rattus norvegicus
  V$NF1 Q6
atttggctcagtgacaga

SEQ ID NO: 330 - DNA, 11 bp, Rattus norvegicus
  V$AP1FJ Q2
agtgacagagc

SEQ ID NO: 331 - DNA, 10 bp, Rattus norvegicus
  V$AP4 Q5
cccagctccg

SEQ ID NO: 332 - DNA, 15 bp, Rattus norvegicus
  V$ISRE 01
gagtttcagtttgcg

SEQ ID NO: 333 - DNA, 10 bp, Rattus norvegicus
  V$VBP 01
gttacatgaa

SEQ ID NO: 334 - DNA, 10 bp, Rattus norvegicus
  V$VMYB 01
atgaacggaa

SEQ ID NO: 335 - DNA, 10 bp, Rattus norvegicus
  V$NRF2 01
aacggaagat

SEQ ID NO: 336 - DNA, 11 bp, Rattus norvegicus
  V$AP1FJ Q2
gatgacccaac

SEQ ID NO: 337 - DNA, 14 bp, Rattus norvegicus
  V$GATA1 03
gttaggataaccag

SEQ ID NO: 338 - DNA, 10 bp, Rattus norvegicus
  V$GATA2 02
taggataacc

SEQ ID NO: 339 - DNA, 9 bp, Rattus norvegicus
  V$LMO2COM 02
aggataacc

SEQ ID NO: 340 - DNA, 12 bp, Rattus norvegicus
  V$CREB 02
catgtgacgtga

SEQ ID NO: 341 - DNA, 14 bp, Rattus norvegicus
  V$ATF 01
atgtgacgtgagta

SEQ ID NO: 342 - DNA, 12 bp, Rattus norvegicus
  V$CREBP1 Q2
tgtgacgtgagt

SEQ ID NO: 343 - DNA, 8 bp, Rattus norvegicus
  V$CREBP1CJUN 01
tgacgtga

SEQ ID NO: 344 - DNA, 8 bp, Rattus norvegicus
  V$CREB 01
tgacgtga

SEQ ID NO: 345 - DNA, 12 bp, Rattus norvegicus
  V$LMO2COM 01
acccaggtgccc

SEQ ID NO: 346 - DNA, 12 bp, Rattus norvegicus
  V$IK2 01
ggctgggagttc

SEQ ID NO: 347 - DNA, 10 bp, Rattus norvegicus
  V$CREL 01
tgggagttcc

SEQ ID NO: 348 - DNA, 11 bp, Rattus norvegicus
  V$AP1FJ Q2
aatgactccac

SEQ ID NO: 349 - DNA, 16 bp, Rattus norvegicus
  V$FREAC7 01
aaaaaataaaaaggaa

SEQ ID NO: 350 - DNA, 12 bp, Rattus norvegicus
  V$NFAT Q6
aaaaggaaagaa

SEQ ID NO: 351 - DNA, 14 bp, Rattus norvegicus
  V$GATA1 03
ttgtagataaaggg

SEQ ID NO: 352 - DNA, 10 bp, Rattus norvegicus
  V$GATA2 02
gtagataaag

SEQ ID NO: 353 - DNA, 10 bp, Rattus norvegicus
  V$GATA3 02
gtagataaag

SEQ ID NO: 354 - DNA, 9 bp, Rattus norvegicus
  V$LMO2COM 02
tagataaag

SEQ ID NO: 355 - DNA, 8 bp, Rattus norvegicus
  V$MZF1 01
aaggggga

SEQ ID NO: 356 - DNA, 12 bp, Rattus norvegicus
  V$IK2 01
gtctgggagagc

SEQ ID NO: 357 - DNA, 11 bp, Rattus norvegicus
  V$AP1FJ Q2
tgtgaccctga

SEQ ID NO: 358 - DNA, 12 bp, Rattus norvegicus
  V$IK2 01
gagagggatcga

SEQ ID NO: 359 - DNA, 8 bp, Rattus norvegicus
  V$MZF1 01
agagggga

SEQ ID NO: 360 - DNA, 12 bp, Rattus norvegicus
  V$IK2 01
agaggggaacca

SEQ ID NO: 361 - DNA, 16 bp, Rattus norvegicus
  V$TH1E47 01
caaaatgtctggatta

SEQ ID NO: 362 - DNA, 16 bp, Rattus norvegicus
  V$FREAC7 01
attatataaaaaagag

SEQ ID NO: 363 - DNA, 14 bp, Rattus norvegicus
  V$OCT1 06
cactttgatatgtt

SEQ ID NO: 364 - DNA, 14 bp, Rattus norvegicus
  V$GATA1 02
actttgatatgtta

SEQ ID NO: 365 - DNA, 9 bp, Rattus norvegicus
  V$LMO2COM 02
ttgatatgt

SEQ ID NO: 366 - DNA, 16 bp, Rattus norvegicus
  V$BRN2 01
gatatgttaaataggc

SEQ ID NO: 367 - DNA, 11 bp, Rattus norvegicus
  V$DELTAEF1 01
aggcacctcag

SEQ ID NO: 368 - DNA, 18 bp, Rattus norvegicus
  V$CMYB 01
cctagagaccgttgttta

SEQ ID NO: 369 - DNA, 11 bp, Rattus norvegicus
  V$AP1FJ Q2
gatgacctctg

SEQ ID NO: 370 - DNA, 14 bp, Rattus norvegicus
  V$OCT1 06
cagtcttgcatgta

SEQ ID NO: 371 - DNA, 8 bp, Rattus norvegicus
  V$MZF1 01
ctggggga

SEQ ID NO: 372 - DNA, 16 bp, Rattus norvegicus
  V$S8 01
ggcgagaaattagcac

SEQ ID NO: 373 - DNA, 12 bp, Rattus norvegicus
  V$IK2 01
ctaagggacccc

SEQ ID NO: 374 - DNA, 12 bp, Rattus norvegicus
  V$IK2 01
cacagggactca

SEQ ID NO: 375 - DNA, 12 bp, Rattus norvegicus
  V$LMO2COM 01
agacaggtggct

SEQ ID NO: 376 - DNA, 12 bp, Rattus norvegicus
  V$MYOD 01
agacaggtggct

SEQ ID NO: 377 - DNA, 12 bp, Rattus norvegicus
  V$NFAT Q6
ctttggaaacat

SEQ ID NO: 378 - DNA, 12 bp, Rattus norvegicus
  V$NFAT Q6
gtttggaaagtc

SEQ ID NO: 379 - DNA, 7 bp, Rattus norvegicus
  V$NKX25 01
ctaagtg

SEQ ID NO: 380 - DNA, 18 bp, Rattus norvegicus
  V$NF1 Q6
cattggctgtggtttctg

SEQ ID NO: 381 - DNA, 9 bp, Rattus norvegicus
  V$PADS C
tgtggtttc

SEQ ID NO: 382 - DNA, 13 bp, Rattus norvegicus
  V$IK1 01
tgatgggaaaagc

SEQ ID NO: 383 - DNA, 12 bp, Rattus norvegicus
  V$IK2 01
tgatgggaaaag

SEQ ID NO: 384 - DNA, 12 bp, Rattus norvegicus
  V$NFAT Q6
gatgggaaaagc

SEQ ID NO: 385 - DNA, 12 bp, Rattus norvegicus
  V$IK2 01
ctttgggatcct

SEQ ID NO: 386 - DNA, 13 bp, Rattus norvegicus
  V$IK1 01
ctctgggaatcgg

SEQ ID NO: 387 - DNA, 12 bp, Rattus norvegicus
  V$IK2 01
ctctgggaatcg

SEQ ID NO: 388 - DNA, 10 bp, Rattus norvegicus
  V$AP4 Q5
aacagcagct

SEQ ID NO: 389 - DNA, 10 bp, Rattus norvegicus
  V$AP4 Q5
agcagctgct

SEQ ID NO: 390 - DNA, 15 bp, Rattus norvegicus
  V$HNF3B 01
gcaaatgtttccttg

SEQ ID NO: 391 - DNA, 12 bp, Rattus norvegicus
  V$HFH2 01
aaatgtttcctt

SEQ ID NO: 392 - DNA, 8 bp, Rattus norvegicus
  V$MZF1 01
ccagggga

SEQ ID NO: 393 - DNA, 10 bp, Rattus norvegicus
  V$AP4 Q5
cacagcagcc

SEQ ID NO: 394 - DNA, 10 bp, Rattus norvegicus
  V$AP4 Q5
aacagctcca

SEQ ID NO: 395 - DNA, 11 bp, Rattus norvegicus
  V$DELTAEF1 01
ctgcacctagc

SEQ ID NO: 396 - DNA, 10 bp, Rattus norvegicus
  V$AP4 Q5
tccagctgtg

SEQ ID NO: 397 - DNA, 12 bp, Rattus norvegicus
  V$IK2 01
aactgggaggta

SEQ ID NO: 398 - DNA, 8 bp, Rattus norvegicus
  V$MZF1 01
ctcgggga

SEQ ID NO: 399 - DNA, 12 bp, Rattus norvegicus
  V$IK2 01
ctcggggatttc

SEQ ID NO: 400 - DNA, 12 bp, Rattus norvegicus
  V$IK2 01
ttctgggaggct

SEQ ID NO: 401 - DNA, 18 bp, Rattus norvegicus
  V$NF1 Q6
acttggctgtctgtaggc

SEQ ID NO: 402 - DNA, 12 bp, Mus musculus
  V$IK2 01
acttgggaggca

SEQ ID NO: 403 - DNA, 24 bp, Mus musculus
  V$GFI1 01
aaaaaaaaaatcaaatttaatact

SEQ ID NO: 404 - DNA, 12 bp, Mus musculus
  V$SRY 02
tctaacaaaacc

SEQ ID NO: 405 - DNA, 9 bp, Mus musculus
  V$LMO2COM 02
ttgatattc

SEQ ID NO: 406 - DNA, 16 bp, Mus musculus
  V$ARNT 01
atattcacgtgctaaa

SEQ ID NO: 407 - DNA, 14 bp, Mus musculus
  V$USF 01
tattcacgtgctaa

SEQ ID NO: 408 - DNA, 14 bp, Mus musculus
  V$MAX 01
tattcacgtgctaa

SEQ ID NO: 409 - DNA, 12 bp, Mus musculus
  V$NMYC 01
attcacgtgcta

SEQ ID NO: 410 - DNA, 12 bp, Mus musculus
  V$MYCMAX 02
attcacgtgcta

SEQ ID NO: 411 - DNA, 10 bp, Mus musculus
  V$USF Q6
ttcacgtgct

SEQ ID NO: 412 - DNA, 8 bp, Mus musculus
  V$USF C
tcacgtgc

SEQ ID NO: 413 - DNA, 12 bp, Mus musculus
  V$NFAT Q6
gttaggaaaata

SEQ ID NO: 414 - DNA, 24 bp, Mus musculus
  V$GFI1 01
aacatacaaatctgagccacggtg

SEQ ID NO: 415 - DNA, 18 bp, Mus musculus
  V$NF1 Q6
cattggcattgcgtgtca

SEQ ID NO: 416 - DNA, 16 bp, Mus musculus
  V$AHRARNT 01
ttggcattgcgtgtca

SEQ ID NO: 417 - DNA, 13 bp, Mus musculus
  V$TCF11 01
gtcatggacatgc

SEQ ID NO: 418 - DNA, 14 bp, Mus musculus
  V$CEBPB 01
aagttgtgcaatgt

SEQ ID NO: 419 - DNA, 11 bp, Mus musculus
  V$AP1FJ Q2
tgtgacatctg

SEQ ID NO: 420 - DNA, 11 bp, Mus musculus
  V$DELTAEF1 01
aagcaccttaa

SEQ ID NO: 421 - DNA, 14 bp, Mus musculus
  V$GATA1 02
cacttgatattgaa

SEQ ID NO: 422 - DNA, 9 bp, Mus musculus
  V$LMO2COM 02
ttgatattg

SEQ ID NO: 423 - DNA, 16 bp, Mus musculus
  V$S8 01
gatattgaattaagtt

SEQ ID NO: 424 - DNA, 15 bp, Mus musculus
  V$HNF3B 01
ttttttgtttgtttg

SEQ ID NO: 425 - DNA, 13 bp, Mus musculus
  V$HFH8 01
ttttgtttgtttg

SEQ ID NO: 426 - DNA, 12 bp, Mus musculus
  V$HFH2 01
ttttgtttgttt

SEQ ID NO: 427 - DNA, 13 bp, Mus musculus
  V$HFH3 01
ttttgtttgtttg

SEQ ID NO: 428 - DNA, 15 bp, Mus musculus
  V$HNF3B 01
ttgtttgtttgtttt

SEQ ID NO: 429 - DNA, 12 bp, Mus musculus
  V$HFH2 01
gtttgtttgttt

SEQ ID NO: 430 - DNA, 13 bp, Mus musculus
  V$HFH3 01
gtttgtttgtttt

SEQ ID NO: 431 - DNA, 13 bp, Mus musculus
  V$HFH8 01
gtttgtttgtttt

SEQ ID NO: 432 - DNA, 13 bp, Mus musculus
  V$HFH3 01
gtttgtttttgtt

SEQ ID NO: 433 - DNA, 16 bp, Mus musculus
  V$TH1E47 01
ttttgtttctggctgt

SEQ ID NO: 434 - DNA, 12 bp, Mus musculus
  V$LMO2COM 01
agccaggtgtgg

SEQ ID NO: 435 - DNA, 13 bp, Mus musculus
  V$TCF11 01
gtcatcccagcac

SEQ ID NO: 436 - DNA, 11 bp, Mus musculus
  V$AP1FJ Q2
tctgacacaga

SEQ ID NO: 437 - DNA, 14 bp, Mus musculus
  V$CEBPB 01
ggttgatgcaagtc

SEQ ID NO: 438 - DNA, 13 bp, Mus musculus
  V$RORA1 01
tgttccaggtcag

SEQ ID NO: 439 - DNA, 12 bp, Mus musculus
  V$SRY 02
acaaacaaaaat

SEQ ID NO: 440 - DNA, 24 bp, Mus musculus
  V$GFI1 01
caaacaaaaatcctagagaaactc

SEQ ID NO: 441 - DNA, 13 bp, Mus musculus
  V$TCF11 01
gtcattgtggccc

SEQ ID NO: 442 - DNA, 12 bp, Mus musculus
  V$NFAT Q6
cataggaaacag

SEQ ID NO: 443 - DNA, 11 bp, Mus musculus
  V$AP1FJ Q2
ggtgaccatag

SEQ ID NO: 444 - DNA, 21 bp, Mus musculus
  V$STAF 02
ctttcccatcatccagagcct

SEQ ID NO: 445 - DNA, 13 bp, Mus musculus
  V$IK1 01
cctagggaacact

SEQ ID NO: 446 - DNA, 12 bp, Mus musculus
  V$IK2 01
cctagggaacac

SEQ ID NO: 447 - DNA, 11 bp, Mus musculus
  V$AP1FJ Q2
cttgacttcca

SEQ ID NO: 448 - DNA, 11 bp, Mus musculus
  V$DELTAEF1 01
gaacaccttgt

SEQ ID NO: 449 - DNA, 11 bp, Mus musculus
  V$DELTAEF1 01
tgccacctgtg

SEQ ID NO: 450 - DNA, 10 bp, Mus musculus
  V$MYOD Q6
gccacctgtg

SEQ ID NO: 451 - DNA, 13 bp, Mus musculus
  V$GATA1 04
aaatgatattcag

SEQ ID NO: 452 - DNA, 9 bp, Mus musculus
  V$LMO2COM 02
atgatattc

SEQ ID NO: 453 - DNA, 11 bp, Mus musculus
  V$DELTAEF 01
ccacacctgct

SEQ ID NO: 454 - DNA, 10 bp, Mus musculus
  V$MYOD Q6
cacacctgct

SEQ ID NO: 455 - DNA, 18 bp, Mus musculus
  V$NF1 Q6
atttggcatctcagaagc

SEQ ID NO: 456 - DNA, 12 bp, Mus musculus
  V$SRY 02
aaaaacaagaag

SEQ ID NO: 457 - DNA, 18 bp, Mus musculus
  V$RFX1 02
aggacaccagtgcaacca

SEQ ID NO: 458 - DNA, 13 bp, Mus musculus
  V$TCF11 01
gtcacgttgcctg

SEQ ID NO: 459 - DNA, 13 bp, Mus musculus
  V$TCF11 01
gtcatatccagtg

SEQ ID NO: 460 - DNA, 11 bp, Mus musculus
  V$DELTAEF1 01
tcacacctgat

SEQ ID NO: 461 - DNA, 10 bp, Mus musculus
  V$MYOD Q6
cacacctgat

SEQ ID NO: 462 - DNA, 16 bp, Mus musculus
  V$TH1E47 01
cactgggtctggcaaa

SEQ ID NO: 463 - DNA, 11 bp, Mus musculus
  V$DELTAEF1 01
ccacacctcat

SEQ ID NO: 464 - DNA, 7 bp, Mus musculus
  V$NKX25 01
tgaagtg

SEQ ID NO: 465 - DNA, 16 bp, Mus musculus
  V$NFY 01
tgagcccaatgggata

SEQ ID NO: 466 - DNA, 12 bp, Mus musculus
  V$IK2 01
caatgggatagt

SEQ ID NO: 467 - DNA, 14 bp, Mus musculus
  V$GATA1 02
aatgggatagtatg

SEQ ID NO: 468 - DNA, 7 bp, Mus musculus
  V$NKX25 01
tgaagtg

SEQ ID NO: 469 - DNA, 18 bp, Mus musculus
  V$CMYB 01
cactgctgctgttgacat

SEQ ID NO: 470 - DNA, 12 bp, Mus musculus
  V$IK2 01
aactgggaccac

SEQ ID NO: 471 - DNA, 11 bp, Mus musculus
  V$DELTAEF1 01
agccacctacc

SEQ ID NO: 472 - DNA, 10 bp, Mus musculus
  V$NRF2 01
acaggaagtg

SEQ ID NO: 473 - DNA, 12 bp, Mus musculus
  V$IK2 01
gacagggaaggg

SEQ ID NO: 474 - DNA, 9 bp, Mus musculus
  V$LMO2COM 02
gtgatacct

SEQ ID NO: 475 - DNA, 7 bp, Mus musculus
  V$NKX25 01
gtaagtg

SEQ ID NO: 476 - DNA, 15 bp, Mus musculus
  V$E47 01
ggtgcaggtggcttc

SEQ ID NO: 477 - DNA, 12 bp, Mus musculus
  V$MYOD 01
gtgcaggtggct

SEQ ID NO: 478 - DNA, 12 bp, Mus musculus
  V$LMO2COM 01
gtgcaggtggct

SEQ ID NO: 479 - DNA, 12 bp, Mus musculus
  V$NFAT Q6
tagaggaaaagc

SEQ ID NO: 480 - DNA, 14 bp, Mus musculus
  V$GATA1 02
gtgatgatagaaga

SEQ ID NO: 481 - DNA, 12 bp, Mus musculus
  V$NFAT Q6
agtaggaaaaga

SEQ ID NO: 482 - DNA, 10 bp, Mus musculus
  V$AP4 Q5
ttcagcagtg

SEQ ID NO: 483 - DNA, 8 bp, Mus musculus
  V$MZF1 01
ctggggga

SEQ ID NO: 484 - DNA, 12 bp, Mus musculus
  V$IK2 01
ctgggggaagga

SEQ ID NO: 485 - DNA, 11 bp, Mus musculus
  V$AP1 Q2
gatgactggtg

SEQ ID NO: 486 - DNA, 12 bp, Mus musculus
  V$IK2 01
agtagggatgga

SEQ ID NO: 487 - DNA, 10 bp, Mus musculus
  V$AP4 Q5
cccagctgag

SEQ ID NO: 488 - DNA, 12 bp, Mus musculus
  V$IK2 01
gagagggactgg

SEQ ID NO: 489 - DNA, 12 bp, Mus musculus
  V$IK2 01
aggagggatggg

SEQ ID NO: 490 - DNA, 12 bp, Mus musculus
  V$IK2 01
gggtgggaggcc

SEQ ID NO: 491 - DNA, 12 bp, Mus musculus
  V$CREB 02
aggatgacgaca

SEQ ID NO: 492 - DNA, 11 bp, Mus musculus
  V$AP1FJ Q2
gatgacgacaa

SEQ ID NO: 493 - DNA, 10 bp, Mus musculus
  V$GATA3 03
agagatcgta

SEQ ID NO: 494 - DNA, 8 bp, Mus musculus
  V$MZF1 01
tcagggga

SEQ ID NO: 495 - DNA, 13 bp, Mus musculus
  V$TCF11 01
gtcatggatgctt

SEQ ID NO: 496 - DNA, 8 bp, Mus musculus
  V$NKX25 02
cttaattc

SEQ ID NO: 497 - DNA, 10 bp, Mus musculus
  V$AP4 Q5
accagctgag

SEQ ID NO: 498 - DNA, 14 bp, Mus musculus
  V$GATA1 02
agagagatacaagg

SEQ ID NO: 499 - DNA, 10 bp, Mus musculus
  V$GATA2 03
agagatacaa

SEQ ID NO: 500 - DNA, 9 bp, Mus musculus
  V$LMO2COM 02
gagatacaa

SEQ ID NO: 501 - DNA, 12 bp, Mus musculus
  V$IK2 01
gtttgggaggtg

SEQ ID NO: 502 - DNA, 9 bp, Mus musculus
  V$LYF1 01
tttgggagg

SEQ ID NO: 503 - DNA, 14 bp, Mus musculus
  V$GATA1 02
ttggtgatagcaga

SEQ ID NO: 504 - DNA, 9 bp, Mus musculus
  V$LMO2COM 02
gtgatagca

SEQ ID NO: 505 - DNA, 7 bp, Mus musculus
  V$NKX25 01
tgaagtg

SEQ ID NO: 506 - DNA, 14 bp, Mus musculus
  V$GATA1 02
tttaggataatgaa

SEQ ID NO: 507 - DNA, 9 bp, Mus musculus
  V$LMO2COM 02
aggataatg

SEQ ID NO: 508 - DNA, 12 bp, Mus musculus
  V$SRY 02
aaaaacaagaac

SEQ ID NO: 509 - DNA, 13 bp, Mus musculus
  V$TCF11 01
gtcatgtgatctg

SEQ ID NO: 510 - DNA, 13 bp, Mus musculus
  V$TCF11 01
gtcatgtgatctg

SEQ ID NO: 511 - DNA, 11 bp, Mus musculus
  V$DELTAEF1 01
taacacctccg

SEQ ID NO: 512 - DNA, 12 bp, Mus musculus
  V$IK2 01
ggctgggagtta

SEQ ID NO: 513 - DNA, 11 bp, Mus musculus
  V$AP1FJ Q2
gatgactccac

SEQ ID NO: 514 - DNA, 11 bp, Mus musculus
  V$CP2 01
gctccatccag

SEQ ID NO: 515 - DNA, 16 bp, Mus musculus
  V$FREAC7 01
aagaaataaaaaggaa

SEQ ID NO: 516 - DNA, 12 bp, Mus musculus
  V$NFAT Q6
aaaaggaaagaa

SEQ ID NO: 517 - DNA, 14 bp, Mus musculus
  V$GATA1 03
ttgtagataaaggg

SEQ ID NO: 518 - DNA, 10 bp, Mus musculus
  V$GATA2 02
gtagataaag

SEQ ID NO: 519 - DNA, 10 bp, Mus musculus
  V$GATA3 02
gtagataaag

SEQ ID NO: 520 - DNA, 9 bp, Mus musculus
  V$LMO2COM 02
tagataaag

SEQ ID NO: 521 - DNA, 12 bp, Mus musculus
  V$IK2 01
gtctgggagagt

SEQ ID NO: 522 - DNA, 8 bp, Mus musculus
  V$MZF1 01
tgtgggga

SEQ ID NO: 523 - DNA, 8 bp, Mus musculus
  V$MZF1 01
agagggga

SEQ ID NO: 524 - DNA, 12 bp, Mus musculus
  V$IK2 01
agaggggaacca

SEQ ID NO: 525 - DNA, 12 bp, Mus musculus
  V$IK2 01
ggtagggaacca

SEQ ID NO: 526 - DNA, 10 bp, Mus musculus
  V$GATA3 03
atagattata

SEQ ID NO: 527 - DNA, 12 bp, Mus musculus
  V$IK2 01
tatagggaagag

SEQ ID NO: 528 - DNA, 16 bp, Mus musculus
  V$TH1E47 01
aagagcctctggcaga

SEQ ID NO: 529 - DNA, 14 bp, Mus musculus
  V$GATA1 02
ttcaggatagaggg

SEQ ID NO: 530 - DNA, 14 bp, Mus musculus
  V$GATA1 03
ttcaggatagaggg

SEQ ID NO: 531 - DNA, 13 bp, Mus musculus
  V$GATA1 04
tcaggatagaggg

SEQ ID NO: 532 - DNA, 9 bp, Mus musculus
  V$LMO2COM 02
aggatagag

SEQ ID NO: 533 - DNA, 12 bp, Mus musculus
  V$IK2 01
ggtagggattga

SEQ ID NO: 534 - DNA, 12 bp, Mus musculus
  V$IK2 01
tgctgggagaac

SEQ ID NO: 535 - DNA, 13 bp, Mus musculus
  V$RORA1 01
acctagaggtcag

SEQ ID NO: 536 - DNA, 14 bp, Mus musculus
  V$OCT1 06
cactttgacatgtt

SEQ ID NO: 537 - DNA, 16 bp, Mus musculus
  V$BRN2 01
gacatgttaaataggc

SEQ ID NO: 538 - DNA, 12 bp, Mus musculus
  V$SRY 02
taatacaattca

SEQ ID NO: 539 - DNA, 18 bp, Mus musculus
  V$CMYB 01
cctagtggcagttgcttg

SEQ ID NO: 540 - DNA, 15 bp, Mus musculus
  V$BARBIE 01
cctgaaagctggtgg

SEQ ID NO: 541 - DNA, 10 bp, Mus musculus
  V$CREL 01
tggactttcc

SEQ ID NO: 542 - DNA, 10 bp, Mus musculus
  V$NFKAPPAB65 01
tggactttcc

SEQ ID NO: 543 - DNA, 13 bp, Mus musculus
  V$IK1 01
atctgggaaggag

SEQ ID NO: 544 - DNA, 12 bp, Mus musculus
  V$IK2 01
atctgggaagga

SEQ ID NO: 545 - DNA, 16 bp, Mus musculus
  V$S8 01
ggtgagaaattagcac

SEQ ID NO: 546 - DNA, 12 bp, Mus musculus
  V$MYOD 01
agacaggtggct

SEQ ID NO: 547 - DNA, 12 bp, Mus musculus
  V$LMO2COM 01
agacaggtggct

SEQ ID NO: 548 - DNA, 13 bp, Mus musculus
  V$TCF11 01
gtcatttcccttt

SEQ ID NO: 549 - DNA, 12 bp, Mus musculus
  V$NFAT Q6
gtttggaaaatc

SEQ ID NO: 550 - DNA, 24 bp, Mus musculus
  V$GFI1 01
gtttggaaaatcaaggctccaaga

SEQ ID NO: 551 - DNA, 7 bp, Mus musculus
  V$NKX25 01
ctaagtg

SEQ ID NO: 552 - DNA, 11 bp, Mus musculus
  V$DELTAEF1 01
gtgcacctcgg

SEQ ID NO: 553 - DNA, 18 bp, Mus musculus
  V$NF1 Q6
cattggctgtggtttctg

SEQ ID NO: 554 - DNA, 9 bp, Mus musculus
  V$PADS C
tgtggtttc

SEQ ID NO: 555 - DNA, 13 bp, Mus musculus
  V$IK1 01
tgatgggaaaagc

SEQ ID NO: 556 - DNA, 12 bp, Mus musculus
  V$IK2 01
tgatgggaaaag

SEQ ID NO: 557 - DNA, 12 bp, Mus musculus
  V$NFAT Q6
gatgggaaaagc

SEQ ID NO: 558 - DNA, 12 bp, Mus musculus
  V$IK2 01
ctttgggatcct

SEQ ID NO: 559 - DNA, 12 bp, Mus musculus
  V$IK2 01
ctctgggaatcg

SEQ ID NO: 560 - DNA, 13 bp, Mus musculus
  V$IK1 01
ctctgggaatcgg

SEQ ID NO: 561 - DNA, 18 bp, Mus musculus
  V$RFX1 02
tcggagccgtggcaacag

SEQ ID NO: 562 - DNA, 17 bp, Mus musculus
  V$RFX1 01
cggagccgtggcaacag

SEQ ID NO: 563 - DNA, 10 bp, Mus musculus
  V$AP4 Q5
agcagctgct

SEQ ID NO: 564 - DNA, 10 bp, Mus musculus
  V$AP4 Q5
aacagcagct

SEQ ID NO: 565 - DNA, 15 bp, Mus musculus
  V$HNF3B 01
gcaaatgtttccttg

SEQ ID NO: 566 - DNA, 12 bp, Mus musculus
  V$HFH2 01
aaatgtttcctt

SEQ ID NO: 567 - DNA, 8 bp, Mus musculus
  V$MZF1 01
ccagggga

SEQ ID NO: 568 - DNA, 10 bp, Mus musculus
  V$AP4 Q5
cacagcagcc

SEQ ID NO: 569 - DNA, 10 bp, Mus musculus
  V$AP4 Q5
aacagctcca

SEQ ID NO: 570 - DNA, 11 bp, Mus musculus
  V$DELTAEF1 01
ctgcacctagc

SEQ ID NO: 571 - DNA, 10 bp, Mus musculus
  V$AP4 Q5
tccagctgtg

SEQ ID NO: 572 - DNA, 12 bp, Mus musculus
  V$IK2 01
ttctgggaggct

SEQ ID NO: 573 - DNA, 18 bp, Mus musculus
  V$RFX1 02
gggaggctgaagcaacag

SEQ ID NO: 574 - DNA, 14 bp, Mus musculus
  V$CEBPB 01
ggctgaagcaacag

SEQ ID NO: 575 - DNA, 12 bp, Homo sapiens
  V$IK2 01
ttgtgggaggtt

SEQ ID NO: 576 - DNA, 12 bp, Homo sapiens
  V$IK2 01
accagggaaagg

SEQ ID NO: 577 - DNA, 12 bp, Homo sapiens
  V$NFAT Q6
ccagggaaagga

SEQ ID NO: 578 - DNA, 13 bp, Homo sapiens
  V$TCF11 01
gtcattcaggtga

SEQ ID NO: 579 - DNA, 12 bp, Homo sapiens
  V$LMO2COM 01
attcaggtgaga

SEQ ID NO: 580 - DNA, 12 bp, Homo sapiens
  V$IK2 01
aggagggatgga

SEQ ID NO: 581 - DNA, 13 bp, Homo sapiens
  V$RORA1 01
ggctggaggtcac

SEQ ID NO: 582 - DNA, 12 bp, Homo sapiens
  V$IK2 01
ggcagggactgc

SEQ ID NO: 583 - DNA, 8 bp, Homo sapiens
  V$MZF1 01
ggagggga

SEQ ID NO: 584 - DNA, 12 bp, Homo sapiens
  V$IK2 01
tggagggaacag

SEQ ID NO: 585 - DNA, 8 bp, Homo sapiens
  V$MZF1 01
cgggggga

SEQ ID NO: 586 - DNA, 11 bp, Homo sapiens
  V$DELTAEF1 01
cttcacctgca

SEQ ID NO: 587 - DNA, 10 bp, Homo sapiens
  V$MYOD Q6
ttcacctgca

SEQ ID NO: 588 - DNA, 12 bp, Homo sapiens
  V$IK2 01
ggcagggatgga

SEQ ID NO: 589 - DNA, 12 bp, Homo sapiens
  V$IK2 01
ctcagggaagag

SEQ ID NO: 590 - DNA, 10 bp, Homo sapiens
  V$AP4 Q5
agcagccgct

SEQ ID NO: 591 - DNA, 11 bp, Homo sapiens
  V$AP1 Q2
gctgacttgga

SEQ ID NO: 592 - DNA, 12 bp, Homo sapiens
  V$IK2 01
ccttgggagggg

SEQ ID NO: 593 - DNA, 11 bp, Homo sapiens
  V$EVI1 02
ggagaagataa

SEQ ID NO: 594 - DNA, 13 bp, Homo sapiens
  V$GATA1 04
agaagataaggct

SEQ ID NO: 595 - DNA, 10 bp, Homo sapiens
  VGATA3 02
gaagataagg

SEQ ID NO: 596 - DNA, 10 bp, Homo sapiens
  V$GATA2 02
gaagataagg

SEQ ID NO: 597 - DNA, 9 bp, Homo sapiens
  V$LMO2COM 02
aagataagg

SEQ ID NO: 598 - DNA, 16 bp, Homo sapiens
  V$E47 02
acccccaggtgtgggg

SEQ ID NO: 599 - DNA, 12 bp, Homo sapiens
  V$LMO2COM 01
ccccaggtgtgg

SEQ ID NO: 600 - DNA, 8 bp, Homo sapiens
  V$MZF1 01
tgtgggga

SEQ ID NO: 601 - DNA, 12 bp, Homo sapiens
  V$IK2 01
tgtggggaacgg

SEQ ID NO: 602 - DNA, 9 bp, Homo sapiens
  V$VMYB 02
gggaacggc

SEQ ID NO: 603 - DNA, 12 bp, Homo sapiens
  V$IK2 01
gccagggagtcc

SEQ ID NO: 604 - DNA, 12 bp, Homo sapiens
  V$IK2 01
ggccgggacttc

SEQ ID NO: 605 - DNA, 14 bp, Homo sapiens
  V$NFKB Q6
ccgggacttcccct

SEQ ID NO: 606 - DNA, 10 bp, Homo sapiens
  V$CREL 01
cgggacttcc

SEQ ID NO: 607 - DNA, 12 bp, Homo sapiens
  V$NFKB C
cgggacttcccc

SEQ ID NO: 608 - DNA, 10 bp, Homo sapiens
  V$NFKAPPAB 01
gggacttccc

SEQ ID NO: 609 - DNA, 14 bp, Homo sapiens
  V$GATA1 02
ccggtgatactgtt

SEQ ID NO: 610 - DNA, 9 bp, Homo sapiens
  V$LMO2COM 02
gtgatactg

SEQ ID NO: 611 - DNA, 14 bp, Homo sapiens
  V$XFD3 01
tctgttaacaaaga

SEQ ID NO: 612 - DNA, 12 bp, Homo sapiens
  V$SRY 02
gttaacaaagag

SEQ ID NO: 613 - DNA, 12 bp, Homo sapiens
  V$NFAT Q6
cactggaaatgg

SEQ ID NO: 614 - DNA, 15 bp, Homo sapiens
  V$HNF3B 01
cgttttgtttttttt

SEQ ID NO: 615 - DNA, 12 bp, Homo sapiens
  V$HFH2 01
ttttgttttttt

SEQ ID NO: 616 - DNA, 13 bp, Homo sapiens
  V$HFH3 01
ttttgtttttttt

SEQ ID NO: 617 - DNA, 10 bp, Homo sapiens
  V$AP4 Q5
ctcagctcac

SEQ ID NO: 618 - DNA, 7 bp, Homo sapiens
  V$NKX25 01
tcaagtg

SEQ ID NO: 619 - DNA, 12 bp, Homo sapiens
  V$IK2 01
agctgggactac

SEQ ID NO: 620 - DNA, 16 bp, Homo sapiens
  V$AHRARNT 01
gactacaggcgtgcac

SEQ ID NO: 621 - DNA, 18 bp, Homo sapiens
  V$NF1 Q6
tgttggctaggctggtct

SEQ ID NO: 622 - DNA, 24 bp, Homo sapiens
  V$GFI1 01
gaggcagaaatcactttggaggct

SEQ ID NO: 623 - DNA, 14 bp, Homo sapiens
  V$CEBPB 01
ggctaaggcaatgg

SEQ ID NO: 624 - DNA, 12 bp, Homo sapiens
  V$NFAT Q6
agaaggaaatga

SEQ ID NO: 625 - DNA, 13 bp, Homo sapiens
  V$RORA1 01
gttgagaggtcac

SEQ ID NO: 626 - DNA, 11 bp, Homo sapiens
  V$NFE2 01
tgctgagtctt

SEQ ID NO: 627 - DNA, 12 bp, Homo sapiens
  V$NFAT Q6
gaatggaaagca

SEQ ID NO: 628 - DNA, 13 bp, Homo sapiens
  V$GATA1 04
accagatatttgc

SEQ ID NO: 629 - DNA, 9 bp, Homo sapiens
  V$LMO2COM 02
cagatattt

SEQ ID NO: 630 - DNA, 13 bp, Homo sapiens
  V$TCF11 01
gtcactatggcca

SEQ ID NO: 631 - DNA, 7 bp, Homo sapiens
  V$NKX25 01
ccaagtg

SEQ ID NO: 632 - DNA, 10 bp, Homo sapiens
  V$AP4 Q5
atcagctggt

SEQ ID NO: 633 - DNA, 14 bp, Homo sapiens
  V$GKLF 01
aaaaggaaggaggg

SEQ ID NO: 634 - DNA, 12 bp, Homo sapiens
  V$IK2 01
ctttgggaggct

SEQ ID NO: 635 - DNA, 15 bp, Homo sapiens
  V$E47 01
gaggcaggtgaatca

SEQ ID NO: 636 - DNA, 12 bp, Homo sapiens
  V$LMO2COM 01
aggcaggtgaat

SEQ ID NO: 637 - DNA, 13 bp, Homo sapiens
  V$RORA1 01
atcacaaggtcag

SEQ ID NO: 638 - DNA, 16 bp, Homo sapiens
  V$S8 01
tactaaaaattagttg

SEQ ID NO: 639 - DNA, 8 bp, Homo sapiens
  V$MZF1 01
ctggggga

SEQ ID NO: 640 - DNA, 12 bp, Homo sapiens
  V$NFAT Q6
aaaaggaaattc

SEQ ID NO: 641 - DNA, 12 bp, Homo sapiens
  V$NFAT Q6
ttgaggaaatta

SEQ ID NO: 642 - DNA, 16 bp, Homo sapiens
  V$S8 01
ttgaggaaattatgct

SEQ ID NO: 643 - DNA, 7 bp, Homo sapiens
  V$NKX25 01
ctaagtg

SEQ ID NO: 644 - DNA, 12 bp, Homo sapiens
  V$IK2 01
ggtcgggaaggg

SEQ ID NO: 645 - DNA, 8 bp, Homo sapiens
  V$MZF1 01
gaagggga

SEQ ID NO: 646 - DNA, 12 bp, Homo sapiens
  V$IK2 01
gtttgggatgat

SEQ ID NO: 647 - DNA, 16 bp, Homo sapiens
  V$BRN2 01
aacatggttaatacgg

SEQ ID NO: 648 - DNA, 16 bp, Homo sapiens
  V$FREAC7 01
aatatataaatatata

SEQ ID NO: 649 - DNA, 14 bp, Homo sapiens
  V$XFD2 01
tatataaatatata

SEQ ID NO: 650 - DNA, 14 bp, Homo sapiens
  V$XFD1 01
tatataaatatata

SEQ ID NO: 651 - DNA, 15 bp, Homo sapiens
  V$TATA 01
atataaatatatatt

SEQ ID NO: 652 - DNA, 11 bp, Homo sapiens
  V$DELTAEF1 01
ctccacctccc

SEQ ID NO: 653 - DNA, 7 bp, Homo sapiens
  V$NKX25 01
tcaagtg

SEQ ID NO: 654 - DNA, 12 bp, Homo sapiens
  V$IK2 01
agctgggactac

SEQ ID NO: 655 - DNA, 16 bp, Homo sapiens
  V$E47 02
gactacaggtgcccac

SEQ ID NO: 656 - DNA, 12 bp, Homo sapiens
  V$LMO2COM 01
ctacaggtgccc

SEQ ID NO: 657 - DNA, 12 bp, Homo sapiens
  V$MYOD 01
ctacaggtgccc

SEQ ID NO: 658 - DNA, 16 bp, Homo sapiens
  V$E47 02
gacctcaggtgatcca

SEQ ID NO: 659 - DNA, 12 bp, Homo sapiens
  V$MYOD 01
cctcaggtgatc

SEQ ID NO: 660 - DNA, 12 bp, Homo sapiens
  V$LMO2COM 01
cctcaggtgatc

SEQ ID NO: 661 - DNA, 11 bp, Homo sapiens
  V$DELTAEF1 01
atccacctgcc

SEQ ID NO: 662 - DNA, 10 bp, Homo sapiens
  V$MYOD Q6
tccacctgcc

SEQ ID NO: 663 - DNA, 16 bp, Homo sapiens
  V$FREAC7 01
aatttataaacaagaa

SEQ ID NO: 664 - DNA, 14 bp, Homo sapiens
  V$XFD2 01
tttataaacaagaa

SEQ ID NO: 665 - DNA, 15 bp, Homo sapiens
  V$TATA 01
ttataaacaagaatg

SEQ ID NO: 666 - DNA, 12 bp, Homo sapiens
  V$SRY 02
ataaacaagaat

SEQ ID NO: 667 - DNA, 8 bp, Homo sapiens
  V$NKX25 02
attaattg

SEQ ID NO: 668 - DNA, 11 bp, Homo sapiens
  V$AP1FJ Q2
catgacacaca

SEQ ID NO: 669 - DNA, 16 bp, Homo sapiens
  V$FREAC7 01
atagcataaacaggtg

SEQ ID NO: 670 - DNA, 14 bp, Homo sapiens
  V$XFD2 01
agcataaacaggtg

SEQ ID NO: 671 - DNA, 16 bp, Homo sapiens
  V$E47 02
ataaacaggtgtctaa

SEQ ID NO: 672 - DNA, 12 bp, Homo sapiens
  V$MYOD 01
aaacaggtgtct

SEQ ID NO: 673 - DNA, 12 bp, Homo sapiens
  V$LMO2COM 01
aaacaggtgtct

SEQ ID NO: 674 - DNA, 12 bp, Homo sapiens
  V$IK2 01
ctttgggaggcc

SEQ ID NO: 675 - DNA, 15 bp, Homo sapiens
  V$E47 01
gaggcaggtggatca

SEQ ID NO: 676 - DNA, 12 bp, Homo sapiens
  V$LMO2COM 01
aggcaggtggat

SEQ ID NO: 677 - DNA, 11 bp, Homo sapiens
  V$SREBP1 01
gatcacttgag

SEQ ID NO: 678 - DNA, 13 bp, Homo sapiens
  V$RORA1 01
cacttgaggtcag

SEQ ID NO: 679 - DNA, 16 bp, Homo sapiens
  V$T3R 01
acttgaggtcaggagt

SEQ ID NO: 680 - DNA, 11 bp, Homo sapiens
  V$AP1FJ Q2
cctgaccaaca

SEQ ID NO: 681 - DNA, 16 bp, Homo sapiens
  V$S8 01
acacaaaaattagcca

SEQ ID NO: 682 - DNA, 16 bp, Homo sapiens
  V$ARNT 01
ggtggcacgtgcctgt

SEQ ID NO: 683 - DNA, 14 bp, Homo sapiens
  V$MAX 01
gtggcacgtgcctg

SEQ ID NO: 684 - DNA, 14 bp, Homo sapiens
  V$USF 01
gtggcacgtgcctg

SEQ ID NO: 685 - DNA, 12 bp, Homo sapiens
  V$MYCMAX 02
tggcacgtgcct

SEQ ID NO: 686 - DNA, 12 bp, Homo sapiens
  V$NMYC 01
tggcacgtgcct

SEQ ID NO: 687 - DNA, 8 bp, Homo sapiens
  V$USF C
gcacgtgc

SEQ ID NO: 688 - DNA, 12 bp, Homo sapiens
  V$IK2 01
acttgggaggct

SEQ ID NO: 689 - DNA, 12 bp, Homo sapiens
  V$IK2 01
acctgggaggca

SEQ ID NO: 690 - DNA, 12 bp, Homo sapiens
  V$IK2 01
gcctgggagaca

SEQ ID NO: 691 - DNA, 11 bp, Homo sapiens
  V$AP1 Q4
agtgactaagc

SEQ ID NO: 692 - DNA, 16 bp, Homo sapiens
  V$AHRARNT 01
ggggtgtggcgtggtg

SEQ ID NO: 693 - DNA, 12 bp, Homo sapiens
  V$IK2 01
tggtgggatggg

SEQ ID NO: 694 - DNA, 16 bp, Homo sapiens
  V$S8 01
cctgggcaattatcta

SEQ ID NO: 695 - DNA, 16 bp, Homo sapiens
  V$S8 01
attatctaattatcgg

SEQ ID NO: 696 - DNA, 18 bp, Homo sapiens
  V$CMYB 01
ctaattatcggttgtcta

SEQ ID NO: 697 - DNA, 11 bp, Homo sapiens
  V$DELTAEF1 01
tctcacctgta

SEQ ID NO: 698 - DNA, 10 bp, Homo sapiens
  V$MYOD Q6
ctcacctgta

SEQ ID NO: 699 - DNA, 13 bp, Homo sapiens
  V$IK1 01
gtttgggaaagct

SEQ ID NO: 700 - DNA, 12 bp, Homo sapiens
  V$IK2 01
gtttgggaaagc

SEQ ID NO: 701 - DNA, 12 bp, Homo sapiens
  V$NFAT Q6
tttgggaaagct

SEQ ID NO: 702 - DNA, 14 bp, Homo sapiens
  V$OCT1 06
cacacttcaatgcc

SEQ ID NO: 703 - DNA, 11 bp, Homo sapiens
  V$AP1 Q4
cttgactcagg

SEQ ID NO: 704 - DNA, 18 bp, Homo sapiens
  V$NF1 Q6
tgttggcgtcccgcaggc

SEQ ID NO: 705 - DNA, 12 bp, Homo sapiens
  V$AP2 Q6
gtcccgcaggca

SEQ ID NO: 706 - DNA, 10 bp, Homo sapiens
  V$AP4 Q5
ggcagctgct

SEQ ID NO: 707 - DNA, 12 bp, Homo sapiens
  V$IK2 01
gtctgggagaga

SEQ ID NO: 708 - DNA, 11 bp, Homo sapiens
  V$AP1FJ Q2
tgtgactctct

SEQ ID NO: 709 - DNA, 7 bp, Homo sapiens
  V$NKX25 01
tgaagtg

SEQ ID NO: 710 - DNA, 24 bp, Homo sapiens
  V$GFI1 01
acgcctggaatcccagcactttgg

SEQ ID NO: 711 - DNA, 12 bp, Homo sapiens
  V$IK2 01
ctttgggaggcc

SEQ ID NO: 712 - DNA, 15 bp, Homo sapiens
  V$E47 01
gaggcaggtggatga

SEQ ID NO: 713 - DNA, 12 bp, Homo sapiens
  V$LMO2COM 01
aggcaggtggat

SEQ ID NO: 714 - DNA, 12 bp, Homo sapiens
  V$CREB 02
tggatgacgagg

SEQ ID NO: 715 - DNA, 11 bp, Homo sapiens
  V$AP1FJ Q2
gatgacgaggt

SEQ ID NO: 716 - DNA, 13 bp, Homo sapiens
  V$RORA1 01
atgacgaggtcag

SEQ ID NO: 717 - DNA, 11 bp, Homo sapiens
  V$AP1FJ Q2
cctgactctac

SEQ ID NO: 718 - DNA, 16 bp, Homo sapiens
  V$S8 01
atacaacaattagctg

SEQ ID NO: 719 - DNA, 12 bp, Homo sapiens
  V$SRY 02
tacaacaattag

SEQ ID NO: 720 - DNA, 10 bp, Homo sapiens
  V$SOX5 01
acaacaatta

SEQ ID NO: 721 - DNA, 15 bp, Homo sapiens
  V$E47 01
atggcaggtgcctgc

SEQ ID NO: 722 - DNA, 12 bp, Homo sapiens
  V$LMO2COM 01
tggcaggtgcct

SEQ ID NO: 723 - DNA, 12 bp, Homo sapiens
  V$IK2 01
attcgggaggct

SEQ ID NO: 724 - DNA, 12 bp, Homo sapiens
  V$IK2 01
acctgggaggtg

SEQ ID NO: 725 - DNA, 12 bp, Homo sapiens
  V$NFAT Q6
aaaaggaaatga

SEQ ID NO: 726 - DNA, 11 bp, Homo sapiens
  V$AP1FJ Q2
aatgacactga

SEQ ID NO: 727 - DNA, 13 bp, Homo sapiens
  V$GATA1 04
cactgatagttat

SEQ ID NO: 728 - DNA, 9 bp, Homo sapiens
  V$LMO2COM 02
ctgatagtt

SEQ ID NO: 729 - DNA, 12 bp, Homo sapiens
  V$IK2 01
ggctgggacctg

SEQ ID NO: 730 - DNA, 11 bp, Homo sapiens
  V$AP1FJ Q2
gctgacccaga

SEQ ID NO: 731 - DNA, 8 bp, Homo sapiens
  V$MZF1 01
tttgggga

SEQ ID NO: 732 - DNA, 12 bp, Homo sapiens
  V$IK2 01
gttagggactag

SEQ ID NO: 733 - DNA, 10 bp, Homo sapiens
  V$VMYB 01
gaaaacggaa

SEQ ID NO: 734 - DNA, 12 bp, Homo sapiens
  V$CREB 02
gttttgacgtcg

SEQ ID NO: 735 - DNA, 14 bp, Homo sapiens
  V$ATF 01
ttttgacgtcgctg

SEQ ID NO: 736 - DNA, 12 bp, Homo sapiens
  V$CREBP1 Q2
tttgacgtcgct

SEQ ID NO: 737 - DNA, 8 bp, Homo sapiens
  V$CREB 01
tgacgtcg

SEQ ID NO: 738 - DNA, 13 bp, Homo sapiens
  V$TCF11 01
gtcatttgtggag

SEQ ID NO: 739 - DNA, 10 bp, Homo sapiens
  V$AP4 Q5
tgcagctctg

SEQ ID NO: 740 - DNA, 7 bp, Homo sapiens
  V$NKX25 01
gtaagtg

SEQ ID NO: 741 - DNA, 10 bp, Homo sapiens
  V$AP4 Q5
ggcagcagct

SEQ ID NO: 742 - DNA, 7 bp, Homo sapiens
  V$NKX25 01
ctaagtg

SEQ ID NO: 743 - DNA, 9 bp, Homo sapiens
  V$PADS C
agtggtttc

SEQ ID NO: 744 - DNA, 13 bp, Homo sapiens
  V$IK1 01
tgatgggaaaagc

SEQ ID NO: 745 - DNA, 12 bp, Homo sapiens
  V$IK2 01
tgatgggaaaag

SEQ ID NO: 746 - DNA, 12 bp, Homo sapiens
  V$NFAT Q6
gatgggaaaagc

SEQ ID NO: 747 - DNA, 12 bp, Homo sapiens
  V$IK2 01
ctttgggatcct

SEQ ID NO: 748 - DNA, 24 bp, Homo sapiens
  V$GFI1 01
cctctgggaatcagagccgcagca

SEQ ID NO: 749 - DNA, 13 bp, Homo sapiens
  V$IK1 01
ctctgggaatcag

SEQ ID NO: 750 - DNA, 12 bp, Homo sapiens
  V$IK2 01
ctctgggaatca

SEQ ID NO: 751 - DNA, 10 bp, Homo sapiens
  V$AP4 Q5
ggcagctgct

SEQ ID NO: 752 - DNA, 10 bp, Homo sapiens
  V$AP4 Q5
ggcagctgct

SEQ ID NO: 753 - DNA, 12 bp, Homo sapiens
  V$IK2 01
gcccgggacccc

SEQ ID NO: 754 - DNA, 8 bp, Homo sapiens
  V$MZF1 01
ggcgggga

SEQ ID NO: 755 - DNA, 10 bp, Homo sapiens
  V$USF Q6
cacacgagcc

SEQ ID NO: 756 - DNA, 10 bp, Homo sapiens
  V$AP4 Q5
cccagctctc

SEQ ID NO: 757 - DNA, 11 bp, Homo sapiens
  V$NFY Q6
tggccaatgca

SEQ ID NO: 758 - DNA, 10 bp, Homo sapiens
  V$AP4 Q5
cccagctgtg

SEQ ID NO: 759 - DNA, 11 bp, Homo sapiens
  V$DELTAEF1 01
actcacctctc

SEQ ID NO: 760 - DNA, 10 bp, Homo sapiens
  V$VMYB 01
gaaaacgggg

SEQ ID NO: 761 - DNA, 11 bp, Homo sapiens
  V$DELTAEF1 01
aagcacctggg

SEQ ID NO: 762 - DNA, 10 bp, Homo sapiens
  V$MYOD Q6
agcacctggg

SEQ ID NO: 763 - DNA, 12 bp, Homo sapiens
  V$IK2 01
acctgggaggtg

SEQ ID NO: 764 - DNA, 10 bp, Homo sapiens
  V$AP4 Q5
atcagccgtc

SEQ ID NO: 765 - DNA, 8 bp, Homo sapiens
  V$MZF1 01
tcggggga

SEQ ID NO: 766 - DNA, 10 bp, Homo sapiens
  V$AP4 Q5
tgcagctgct

SEQ ID NO: 767 - DNA, 10 bp, Homo sapiens
  V$CETS1P54 01
gccggaggtt

SEQ ID NO: 768 - DNA, 12 bp, Homo sapiens
  V$IK2 01
ggctgggaagca

SEQ ID NO: 769 - DNA, 13 bp, Homo sapiens
  V$IK1 01
ggttgggaagccc

SEQ ID NO: 770 - DNA, 12 bp, Homo sapiens
  V$IK2 01
ggttgggaagcc

SEQ ID NO: 771 - DNA, 12 bp, Homo sapiens
  V$IK2 01
agaagggactac

SEQ ID NO: 772 - DNA, 8 bp, Homo sapiens
  V$MZF1 01
gcagggga

SEQ ID NO: 773 - DNA, 8 bp, Homo sapiens
  V$MZF1 01
attgggga

SEQ ID NO: 774 - DNA, 16 bp, Homo sapiens
  V$TH1E47 01
tatctgttctggcttt

SEQ ID NO: 775 - DNA, 10 bp, Homo sapiens
  V$GATA3 03
tcagatcata

SEQ ID NO: 776 - DNA, 24 bp, Homo sapiens
  V$GFI1 01
tttgcctaaatcacggtagaagtt

SEQ ID NO: 777 - DNA, 11 bp, Homo sapiens
  V$AP1FJ Q2
ggtgacaggtg

SEQ ID NO: 778 - DNA, 12 bp, Homo sapiens
  V$LMO2COM 01
tgacaggtgcat

SEQ ID NO: 779 - DNA, 11 bp, Homo sapiens
  V$AP1FJ Q2
cctgaccctgt

SEQ ID NO: 780 - DNA, 11 bp, Homo sapiens
  V$CP2 01
gcccagcccag

SEQ ID NO: 781 - DNA, 12 bp, Homo sapiens
  V$IK2 01
agtagggaatca